(19) United States
(12) Patent Application Publication
(10) Pub. No.: US 2006/0160201 A1
(43) Pub. Date: Jul. 20, 2006

(54) Three-dimensional structures of HDAC9 and Cabin1 and compound structures and methods related thereto

(76) Inventors: Lin Chen, Boulder, CO (US); Aidong Han, Boulder, CO (US); Ju He, Boulder, CO (US)

Correspondence Address: SHERIDAN ROSS PC, 1560 BROADWAY, SUITE 1200, DENVER, CO, 80202, US

(21) Appl. No.: 11/251,643

(22) Filed: Oct. 14, 2005

Related U.S. Application Data
(63) Continuation-in-part of application No. PCT/US04/11744, filed on Apr. 16, 2004.
(60) Provisional application No. 60/463,547, filed on Apr. 16, 2003.

(51) Int. Cl. G06F 19/00; C12N 9/16; A61K 31/404

(52) U.S. Cl. 435/196; 514/419; 702/19

(57) ABSTRACT

Disclosed are the three-dimensional structures of two complexes: a Cabin1/MEF2B/DNA complex and a MITR/MEF2B/DNA complex. Also disclosed are methods of using such structures and models derived therefrom in structure-based design methods to identify regulators of the interaction of MEF2 with its cognate ligands/corepressors, to compounds that can be designed or identified based on the knowledge of such structures and models, and to methods of using such compounds in therapeutic methods. Also disclosed are peptide and non-peptide regulatory compounds that regulate, and preferably inhibit, MEF2.

### CROSS-REFERENCE TO RELATED APPLICATIONS

This application is a continuation-in-part of copending PCT Application Serial No. PCT/US04/011744, filed Apr. 16, 2004, which designates the United States and was published in English, and which claims the benefit of priority under 35 U.S.C. §119(e) from U.S. Provisional Application Ser. No. 60/463,547, filed Apr. 16, 2003. The entire disclosure of each of PCT Application Serial No. PCT/US04/011744 and U.S. Provisional Application Ser. No. 60/463,547 is incorporated herein by reference.

### REFERENCE TO SEQUENCE LISTING

This application contains a Sequence Listing submitted on a compact disc, in duplicate. Each of the two compact discs, which are identical to each other pursuant to 37 CFR §1.52(e)(4), contains the following file: “Sequence Listing”, having a size in bytes of 155 KB, recorded on Oct. 14, 2005. The information contained on the compact disc is hereby incorporated by reference in its entirety pursuant to 37 CFR §1.77(b)(4).

### GOVERNMENT SUPPORT

This invention was made with government support under Grant No. AI49905 and Grant No. NIH R01 HL076334, each awarded by the National Institutes of Health. The government has certain rights to this invention.

### FIELD OF THE INVENTION

This invention generally relates to three-dimensional structures of histone deacetylase 9 (HDAC9) and Cabin1, to methods of using such structures to design and identify compounds that regulate the interaction of HDACs and/or Cabin1 with their ligands, and to compounds designed or identified thereby that regulate the interaction of HDACs and/or Cabin1 with their ligands.

### BACKGROUND OF THE INVENTION

Myocyte enhancer factor-2 (MEF2) plays critical roles in the development and adaptive responses of the muscle, immune and nervous systems. MEF2 has been implicated as a key regulator of hypertrophic responses in heart muscle cells. Heart hypertrophy induced by pathological stimuli can lead to heart failure in many forms of cardiovascular diseases. MEF2 generally defines a family of transcription factors with four members: MEF2A, MEF2B, MEF2C and MEF2D. The importance of their function has been demonstrated in detail through the use of murine and Drosophila genetics. MEF2, in skeletal muscles where it was initially identified, together with myogenic basic helix-loop-helix transcription factors such as MyoD, promotes and maintains myogenesis (Molkentin, 1996). MEF2 is now known to be a general transcriptional factor in many other cell types. For instance, MEF2 is one of the important transcriptional factors for mediating calcium signaling in lymph system development (Youn, 2000;Youn, 2000). MEF2A, one member of the MEF2 family, has recently been coined as the “heart attack gene” because a mutation in this protein is linked to coronary artery disease (CAD) and myocardial infarction (MI). These findings underlie a critical role of MEF2 in human heart diseases.

Inside cells, the action of MEF2 includes three distinct steps: (i) transcriptional repression; (ii) calcium-dependent de-repression; and (iii) transcription activation. Transcriptional repression by MEF2 depends on its association with a variety of transcriptional co-repressors with intrinsic or associated histone deacetylase (HDAC) activity. In muscle cells, MEF2 binds directly to class II HDACs such as HDAC4, HDAC5, and HDAC9 and inhibits the expression of specific genes involved in the development and adaptive responses of muscle. Mice in which HDAC5 or HDAC9 has been knocked out showed increased sensitivity to hypertrophic stimuli, suggesting important roles of class II HDACs in heart hypertrophy (Zhang et al., 2002). These and other data indicate that MEF2 and Class II histone deacetylases (HDACs), and particularly HDAC5 and HDAC9, are key mediators of hypertrophic signals in cardiomyocytes. Extensive biochemical and functional studies have suggested that the MEF2/class II HDAC pathway is potential therapeutic target for heart hypertrophy.

In response to specific cellular signals, MEF2 turns on distinct programs by association with a variety of transcriptional activators and co-activators (McKinsey et al., 2002). MEF2 has a highly conserved N-terminal region (residues 2-93, with reference to SEQ ID NO:2), consisting of the well-characterized MADS-box and a MEF2-specific domain (Shore et al., 1995). The MADS-box/MEF2 domain is remarkably rich in function, mediating DNA binding, dimerization, and protein-protein interactions with a myriad of MEF2 transcription partners (see McKinsey et al., 2002 for review). It has been shown that the MADS-box/MEF2 domain in MEF2 is necessary and sufficient to bind with a small motif of-class II HDACs (Miska et al., 1999; Sparrow et al., 1999; Youn et al., 1999; Youn et al., 2000).

Histone acetylase and deacetylase are components of essential regulatory mechanisms for gene expression. Mammalian HDACs are categorized into three classes by homology to yeast hda1 and Rpd3. Class II histone deacetylases (HDACs), including HDAC4, HDAC5, HDAC7 and HDAC9, have unique N-terminal regulatory domains that are not found in other HDACs, in addition to a catalytic domain. This class of HDACs targets downstream transcriptional factors such as MEF2 and recruits class I HDACs or other co-factors to enhance their suppression. Notably, MEF2 interacting transcriptional repressor (MITR), an alternative spliced form of HDAC9, does not have a catalytic domain but nonetheless can function well in vivo. Thus, class II HDACs have been the subject of extensive investigation. For example, as mentioned above, HDAC9 has been shown to suppress MEF2-dependent gene expression induced by hypertrophic signals in cardiomyocytes (Zhang et al., 2002). Although the functional significance of MEF2-mediated transcription repression by HDACs has been well established, the mechanisms of sequence-specific recruitment of HDACs by MEF2 are not clearly defined.

Small molecule inhibitors of HDACs, such as trichostatin A (TSA) and butyrate, have been shown to block muscle cell differentiation in culture studies. These seemingly contradictory data suggest that HDACs may have multiple roles in muscle development. There is also evidence that HDAC inhibitors suppress fetal cardiac gene expression in cultured cardiac myocytes stimulated with hypertrophic agonists, again a paradoxical result, given the fact that MEF2/HDAC complexes are known to repress hypertrophic gene expression. These results suggest that HDACs serve as both positive and negative regulators of cardiomyocyte hypertrophy and imply a utility for HDAC inhibitors in treating heart failure. The small molecule inhibitors used in the above studies bind the active site of the deacetylase domain common to all HDAC proteins. The broad inhibition of HDACs with these compounds may explain the complex cellular responses observed thus far.

One way to further dissect the in vivo functions of HDACs and explore the full clinical potential of HDAC inhibition would be to develop inhibitors targeting the enzymatic activity of a specific member of the HDAC family. Considering the conserved nature of the active site of HDACs, this may be very challenging. An alternative approach is to target the steps involved in the interaction between HDACs and their regulators. The recruitment of class II HDACs by MEF2 is a very attractive target for the development of more specific inhibitors of the interaction between MEF2 and its corepressors or other ligands, including HDACs.

Therefore, there is a need in the art for structural information regarding MEF2/co-repressor complexes that can aid in the design of small molecule inhibitors to modulate gene expression in the muscle and immune systems, as well as for lead compounds and inhibitors that regulate the interaction between MEF2 family members and their ligands/corepressors. These inhibitors may be used to treat a variety of human diseases, and in particular hypertrophic cardiomyopathy (HCM), autoimmune diseases and transplant rejection.

### SUMMARY OF THE INVENTION

One embodiment of the present invention relates to an isolated peptide comprising an amino acid sequence represented by SEQ ID NO:22. The peptide is less than about 50 amino acids in length, selectively binds to MEF2, and regulates the activity of MEF2. In one aspect, the peptide consists essentially of less than 30 amino acids of an amino acid sequence selected from: SEQ ID NO:4, SEQ ID NO:6, SEQ ID NO:8, SEQ ID NO:10, SEQ ID NO:12 or SEQ ID NO:14. In one aspect, the peptide comprises an amino acid sequence in the first 200-250 N-terminal amino acids of an HDAC. In another aspect, the peptide comprises an amino acid sequence in the first 160 N-terminal amino acid residues of an HDAC. In yet another embodiment, the peptide comprises an amino acid sequence comprising or aligning with amino acids represented by any one of SEQ ID NO:18, SEQ ID NO:19 or SEQ ID NO:20. In another embodiment, the peptide comprises an amino acid sequence comprising or aligning with amino acid residues (with respect to SEQ ID NO:4): Val143, Lys144, Lys146, Leu147, Gln148, Phe150 and Leu151. In another embodiment, the peptide comprises an amino acid sequence comprising or aligning with any one or more of amino acids with respect to SEQ ID NO:4: Val143, Lys144, Lys146, Leu147, Gln148, Phe150, Leu151, and Phe177. In yet another aspect, the peptide comprises an amino acid sequence that binds to a region of MEF2 comprising or aligning with any one or more of amino acids (with respect to SEQ ID NO:2): Gln56, Met62, Asp63, Leu66, Leu67, Tyr69, Thr70, Tyr72, Ser73, Glu74, Pro75, and Ser78. In another aspect, the peptide consists essentially of positions 1-19 of SEQ ID NO:23. In another aspect, the peptide consists essentially of SEQ ID NO:23.

Another embodiment of the invention comprises any of the above-identified peptides linked to an amino acid sequence for transducing the peptide into the nucleus of a cell. In one aspect, the amino acid sequence for transducing the peptide into the nucleus of a cell comprises positions 24-36 of SEQ ID NO:23. In another aspect, the chimeric peptide consists essentially of SEQ ID NO:23.

One embodiment of the present invention relates to a method of structure-based identification of candidate compounds for regulation of interactions of myocyte enhancer factor 2 (MEF2) with its cognate ligands. The method includes the steps of: (a) providing a three dimensional structure of an HDAC, a Cabin1 protein or a MEF2 protein in a conformation from a complex of either HDAC or Cabin1 with MEF2 and DNA; and (b) identifying at least one candidate compound for interacting with the three dimensional structure of an active site in MEF2, HDAC, Cabin1, an HDAC and MEF2 complex, or a Cabin1 and MEF2 complex by performing structure based drug design with the structure of (a). The three dimensional structure of (a) is selected from: (i) a structure defined by atomic coordinates of a three dimensional structure of a crystalline MEF2 region in complex with DNA and a protein selected from the group consisting of Cabin1 and HDAC9 (MITR); (ii) a structure defined by atomic coordinates selected from: (1) atomic coordinates represented by PDB Accession Nos. selected from the group consisting of PDB Accession No. 1TQE (HDAC9/MEF2/DNA) and PDB Accession No. 1N6J (Cabin1/MEF2/DNA); and, (2) atomic coordinates that define a three dimensional structure wherein at least 50% of the structure has an average root-mean-square deviation (RMSD) from backbone atoms in secondary structure elements in at least one domain of a three dimensional structure represented by the atomic coordinates of (1) of equal to or less than about 1.5 Å; (iii) a structure defined by atomic coordinates derived from HDAC9/MEF2/DNA molecules arranged in a crystalline manner in a space group P1 so as to form a unit cell of dimensions a=44.765 Å, b=66.859 Å, c=66.924 Å (alpha=76.656, beta=71.846, gamma=71.799); (iv) a structure defined by atomic coordinates derived from Cabin1/MEF2/DNA molecules arranged in a crystalline manner in a space group P4₁22 so as to form a unit cell of dimensions a=b=70.14 Å and c=151.88 Å; and (v) a structure of MEF2 in complex with an HDAC protein and DNA constructed using as a template the three-dimensional structure of (ii). Protein domains are well known in the art and have been described for the HDAC, MEF2 and Cabin1 proteins of the invention.

In one aspect of this embodiment, the structure of (a) is a structure defined by atomic coordinates that define a three dimensional structure, wherein at least 50% of the structure has an average root-mean-square deviation (RMSD) from backbone atoms in secondary structure elements in the amino acids of a three dimensional structure represented by the atomic coordinates of (1) of equal to or less than about 1.0 Å. In another aspect, the structure of (a) is a structure defined by atomic coordinates that define a three dimensional structure, wherein at least 50% of the structure has an average root-mean-square deviation (RMSD) from backbone atoms in secondary structure elements in the amino acids of a three dimensional structure represented by the atomic coordinates of (1) of equal to or less than about 0.5 Å. In yet another aspect, the structure of (a) is a structure defined by atomic coordinates that define a three dimensional structure, wherein the structure has an average root-mean-square deviation (RMSD) from backbone atoms in secondary structure elements in the amino acids of a three dimensional structure represented by the atomic coordinates of (1) of equal to or less than about 1.5 Å. In another aspect, the structure of (a) is a structure defined by atomic coordinates that define a three dimensional structure, wherein the structure has an average root-mean-square deviation (RMSD) from backbone atoms in secondary structure elements in the amino acids of a three dimensional structure represented by the atomic coordinates of (1) of equal to or less than about 1.0 Å. In yet another aspect, the structure of (a) is a structure defined by atomic coordinates that define a three dimensional structure, wherein the structure has an average root-mean-square deviation (RMSD) from backbone atoms in secondary structure elements in the amino acids of a three dimensional structure represented by the atomic coordinates of (1) of equal to or less than about 0.5 Å.

In one aspect of this embodiment, the step of identifying comprises computational screening of one or more databases of chemical compounds.

In one aspect of this embodiment, the HDAC structure of (v) is the structure of a class II HDAC, including, but not limited to, an HDAC4, an HDAC5, an HDAC7 and an HDAC9.

In one aspect of this embodiment, the candidate compound is a candidate inhibitor of HDAC/MEF2/DNA complex activity or of Cabin1/MEF2/DNA activity. In one aspect, the candidate compound is a candidate enhancer of HDAC/MEF2/DNA complex activity or of Cabin1/MEF2/DNA activity.

In another aspect of this embodiment, the method further includes the steps of: (c) synthesizing the candidate compound identified in (b); and (d) selecting candidate compounds from (c) that regulate the interaction of MEF2 with a MEF2 ligand. In one aspect, the MEF2 ligand is selected from the group consisting of an HDAC, Cabin1 and p300. An HDAC is preferably a class II HDAC, and more preferably an HDAC is selected from HDAC4, an HDAC5, an HDAC7, and an HDAC9. The step of selecting can include selecting compounds that bind to a protein selected from the group consisting of an HDAC, Cabin1 and MEF2. In another aspect, the step of selecting comprises selecting candidate compounds that bind to MEF2 and inhibit the interaction of MEF2 with the MEF2 ligand. In another aspect, the step of selecting comprises selecting candidate compounds that bind to an HDAC or Cabin1 and inhibit the interaction of MEF2 with the HDAC or Cabin1, respectively. In yet another aspect, the step of selecting comprises selecting candidate compounds that bind to a protein selected from the group consisting of: MEF2, Cabin1, and an HDAC, and inhibit at least one biological activity of the protein.

In one aspect, the step of selecting comprises: (i) contacting the candidate compound synthesized in step (c) with MEF2 or a fragment thereof and a MEF2 ligand or a fragment thereof and with DNA under conditions in which a MEF2-MEF2 ligand-DNA complex can form in the absence of the candidate compound; and (ii) measuring the binding affinity of said MEF2 or fragment thereof to said MEF2 ligand or fragment thereof; wherein a candidate inhibitor compound is selected as a compound that inhibits the binding of MEF2 to its ligand when there is a decrease in the binding affinity of said MEF2 or fragment thereof for said MEF2 ligand or fragment thereof, as compared to in the absence of said candidate compound. In one aspect, the step of selecting comprises using an electrophoresis mobility shift assay (EMSA) to monitor the formation of a ternary complex among MEF2, DNA and the MEF2 ligand in the presence and the absence of the candidate compound, wherein a change in the formation of the complex in the presence of the compound as compared to in the absence of the compound indicates that the compound is a regulator of the interaction of MEF2 with the MEF2 ligand. In either of these aspects, the MEF2 ligand can include, but is not limited to, an HDAC, Cabin1 and p300. The HDAC is preferably a class II HDAC, and more preferably, is selected from an HDAC4, an HDAC5, an HDAC7, and an HDAC9.

The method of Claim 1, wherein the active site comprises at least a portion of the interface between a dimer of MEF2 proteins and HDAC or Cabin1.

In one aspect, the active site in step (b) of the method comprises a ligand groove formed by the H2 helices and a β-sheet comprising the S3 β-strands of a MEF2 dimer. In another aspect, the active site comprises a surface groove formed by -strands S2 and S3 and linkers between S2, H2, and S3 of a MEF2 dimer. In yet another aspect, the active site comprises one or both MEF2S domains of a MEF2 dimer. In another aspect, the active site comprises an amphipathic helix of HDAC or Cabin1 that binds to a hydrophobic ligand groove formed by the H2 helices of a MEF2 dimer. In another aspect, the active site comprises the hydrophobic face of the amphipathic helix of HDAC or Cabin1. In yet another aspect, the active site comprises at least a portion of an amphipathic helix of Cabin1 comprising or aligning with SEQ ID NO:17. In another aspect, the active site comprises at least a portion of an amphipathic helix of Cabin1, comprising or aligning with amino acids Ile2164, Thr2168, Leu2172, Ile2176 and Leu2177, with respect to SEQ ID NO:14. In yet another aspect, the step of identifying comprises identifying candidate compounds for binding to a region of Cabin1 comprising or aligning with any one or more of amino acid residues: Lys2161, Gly2162, Ser2163, Ile2164, Thr2168, Lys2169, Lys2171, Leu2172, Lys2173, Ile2176 Leu2177, Ser2182, Ala2182, Ala2183, and Asn2184, with respect to SEQ ID NO:14. In yet another aspect, the active site comprises a beta sheet like interaction between loop I of MEF2 and the N-terminal tail of HDAC. In another aspect, the active site comprises the first 200-250 N-terminal amino acids of an HDAC. In another aspect, the active site comprises the first 160 N-terminal amino acid residues of an HDAC. In yet another aspect, the active site comprises at least a portion of an amphipathic helix of an HDAC comprising or aligning with amino acids represented by any one of SEQ ID NO:18, SEQ ID NO:19 or SEQ ID NO:20. In yet another aspect, the active site comprises a hydrophobic face of an amphipathic helix of HDAC9 (MITR) comprising or aligning with amino acid residues (with respect to SEQ ID NO:4): Val143, Lys144, Lys146, Leu147, Gln148, Phe150 and Leu151.

In one aspect, the step of identifying comprises identifying candidate compounds for binding to a region of HDAC9 (MITR) comprising or aligning with any one or more of amino acids with respect to SEQ ID NO:4: Va143, Lys144, Lys146, Leu147, Gln148, Phe150, Leu151, and Phe177. In another aspect, the step of identifying comprises identifying candidate compounds for binding to a region of MEF2 comprising or aligning with any one or more of amino acids (with respect to SEQ ID NO:2): Gln56, Met62, Asp63, Leu66, Leu67, Tyr69, Thr70, Tyr72, Ser73, Glu74, Pro75, and Ser78.

The step of performing structure based drug design can include any suitable technique or method, including, but not limited to, computational screening of one or more databases of chemical compound structures to identify candidate compounds which have structures that are predicted to interact with the three dimensional structure of an HDAC or Cabin1 protein in complex with MEF2 and DNA; directed drug design; random drug design and grid-based drug design.

In another embodiment, the present invention provides pharmaceutical compositions with MEF2 binding capability as well as methods of administering these compounds to an animal in need of such treatment. The pharmaceutical compositions include, but are not limited to:

(a) a compound having a formula selected from the group consisting of: 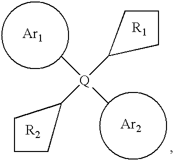
Ar₁—R₂-Q-R₁—Ar₂ and Ar₁—R₁-Q-R₂—Ar₂

or pharmaceutically acceptable salts thereof,

wherein, Ar₁ and Ar₂ are independently C₅-C₁₀ aromatic, C₅-C₁₀ heterocyclic or aralkyl; R₁ and R₂ are independently C₁-C₁₀ alkyl or alkylene; Q is C, C═C, C₁-C₁₀ alkyl or phenyl; and (b) a pharmaceutically acceptable carrier.

### BRIEF DESCRIPTION OF THE DRAWINGS OF THE INVENTION

FIG. 1 is a schematic drawing showing the overall structure of Cabin1 (top) bound to a MEF2 dimer on DNA (bottom).

FIG. 2A is a schematic drawing showing the ligand binding groove formed by the alpha helix H2 and beta strands S1, S2 and S3 of each MEF2 monomer.

FIG. 2B is a schematic drawing showing the hydrophobic nature (the lighter patches around the Cabin1 helix) of the MEF2 ligand-binding pocket.

FIG. 3 is a schematic drawing showing detailed binding interactions between Cabin1 and MEF2.

FIG. 4 is a sequence alignment of the MEF2-binding motifs of Cabin1 (SEQ ID NO:17), HDAC4 (SEQ ID NO:18), HDAC5 (SEQ ID NO:19), HDAC9 (SEQ ID NO:20) and p300 (SEQ ID NO:21).

FIG. 5 is a digital image of the results of an electrophoresis mobility shift assay (EMSA) showing the titration of Cabin1 (2144-2220) to the MEF2B (1-116) dimer/DNA complex.

FIGS. 6A-6C are digital images of the results of a mutational analysis on HDAC4.

FIG. 7A is a sequence alignment of the MEF2-binding motifs of Cabin1 (SEQ ID NO:17), HDAC4 (SEQ ID NO:18), HDAC5 (SEQ ID NO:19), HDAC9 (SEQ ID NO:20) and p300 (SEQ ID NO:21).

FIG. 7B is a digital image of the results of a mutational analysis of the conserved Phe on HDAC9.

FIG. 8 is a schematic drawing of the overall structure of the MITR/MEF2/DNA complex.

FIG. 9 is a schematic drawing showing how the conserved Phe in HDAC9 inserts into a hydrophobic groove on MEF2.

FIG. 10 is a schematic drawing illustrating structure-based strategies for designing MEF2-binding inhibitors.

FIG. 11A is a schematic drawing illustrating the triple helix bundle formed between the amphipathic helix of MITR and two top MEF2 helices.

FIG. 11B is a schematic drawing further illustrating the triple helix bundle formed between the amphipathic helix of MITR and two top MEF2 helices.

FIG. 12A is a schematic drawing illustrating the plasticity of the MEF2 groove.

FIG. 12B is a schematic drawing further illustrating the plasticity of the MEF2 groove.

FIG. 13 is a schematic drawing illustrating a backbone superposition of the HDAC9/MEF2/DNA complex and the Cabin1/MEF2/DNA complex.

FIG. 14 is a schematic drawing illustrating mechanisms of ligand binding to the hydrophobic groove of MEF2 (consensus motif is SEQ ID NO:22).

### DETAILED DESCRIPTION OF THE INVENTION

The present invention generally relates to the determination by the present inventors of the three-dimensional structure of two different MEF2 complexes; to the protein and DNA binding interactions that are uniquely revealed by the determination of these structures; to methods for designing and identifying compounds that bind to and regulate the proteins in the complexes, to the compounds designed or identified by such methods; and to the use of such compounds in therapeutic methods in which regulation of the proteins in the complex can be beneficial. Specifically, the present invention relates to the three-dimensional structures of a Cabin1/MEF2/DNA complex and a MEF2/HDAC9(MITR)/DNA complex, to methods of use thereof, and to regulatory compounds identified based on the elucidation of these structures, as well as the use of such compounds in therapeutic methods.

In one embodiment, the present invention relates to novel peptide regulators of MEF2, and preferably, peptide inhibitors, which are designed based on the structure of the Cabin1/MEF2/DNA complex and the MEF2/HDAC9(MITR)/DNA complex described herein. Such inhibitors can include peptide fragments of any of the MEF2 cognate ligands described herein (any HDAC, Cabin1, p300), and can include homologues of such fragments that bind to MEF2 and inhibit a biological activity of MEF2 (e.g., by acting as a competitive inhibitor of the natural ligand and/or by having an antagonistic effect on at least one biological activity of MEF2 as described herein). Preferably, such inhibitors are able to efficiently enter the cytoplasm of a cell and more preferably, the nucleus of a cell. Such a characteristic can be provided, for example, by linking the inhibitory peptide to a peptide that is capable of transporting the peptide across a cell membrane, such as the nuclear membrane. In one aspect, the inhibitors are labeled with a detectable label. In another aspect, the inhibitors are water soluble. In one embodiment, such a peptide consists essentially of or consists of a sequence having the motif represented herein by SEQ ID NO:22. In another aspect of the invention, an exemplary peptide inhibitor of MEF2 according to the present invention is represented by positions 1-19 of SEQ ID NO:23. In yet another aspect of the invention, an exemplary peptide inhibitor of MEF2 according to the invention is represented by SEQ ID NO:23. In another aspect of the invention, a peptide regulator is a homologue of SEQ ID NO:23.

More particularly, using the structural information provided herein, the present inventors have developed a short peptide that specifically binds MEF2, represented herein as positions 1-19 of SEQ ID NO:23. This peptide has potential applicatiohs in inhibiting pathological gene expression in hypertrophic heart muscle cells, and may also be used in manipulating MEF2-dependent gene expression in a variety of cellular processes, such as the autoimmune response where MEF2-mediated gene expression is undesirably turned on. The peptide was designed based on the inventors' crystal structures of the MEF2/Cabin1/DNA complex and the MEF2/HDAC9/DNA complex. The peptide can bind MEF2 at 0.7 μM Kd and compete efficiently with the ligand, p300. Thus, without being bound by theory, the present inventors believe that this peptide can be used in vivo to selectively inhibit MEF2-mediated gene expression during hypertrophic growth of heart and autoimmune response of the immune systems, where MEF2-controlled genes are turned on by pathological signals. When used at appropriate dosage, this peptide should not affect normal physiological functions of MEF2, because the affinity of MEF2 for class II HDACs is much higher than this peptide. To the best of the present inventors' knowledge, this is the first ever small molecule inhibitor of MEF2 to be developed based on the unique knowledge of the MEF2/co-regulator complexes.

The present inventors disclose herein the crystal structure of Cabin1/MEF2/DNA, which revealed the complete conformation of the N-terminal conserved domain of MEF2, where a MEF2 dimer mediates DNA binding on one side and recruits the Cabin1 ligand on another. Second, in order to further elucidate the MEF2 recruitment mechanism for different ligands, the present inventors have solved the crystal structure of MEF2 with MITR (MEF2 Interacting Transcriptional Repressor, an alternative spliced form of HDAC9) and DNA. These results provide the first detailed understanding of the MEF2 interaction with specific ligands/corepressors at a structural level and reveal potential target sites for molecular drug design.

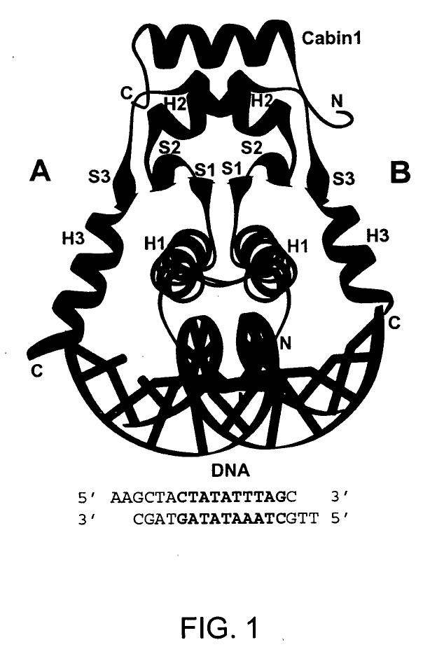

Specifically, the present inventors have obtained diffracting crystals (2.1 Å) of Cabin1 (amino acid residues 2157-2190 of SEQ ID NO:14) bound to MEF2B (amino acid residues 1-93 of SEQ ID NO:2) on DNA (the sequence of the DNA is shown in FIG. 1; represented by SEQ ID NO:15 and SEQ ID NO:16) and have also solved the crystal structure of the Cabin1/MEF2/DNA complex. The methods and the DNA sequence established in the crystallization of the Cabin1/MEF2/DNA complex facilitated the crystallization and resolution of the HDAC9/MEF2/DNA complex, which has been resolved to 2.7 Å. These methods and structures will also be useful for crystallizing MEF2 bound to inhibitors for structural analyses and structure based improvement of the inhibitor design.

The crystal structure of the Cabin1/MEF2/DNA complex solved by the inventors reveals that the Cabin1-binding site on MEF2 bears a resemblance to the antigen peptide binding site of the Major Histocompatibility Complex (MHC). Preliminary studies by the inventors indicate that MEF2 uses the MHC-like domain to recruit other transcription co-regulators (e.g., class II HDACs and p300) by binding small alpha helices in targeted proteins. Thus, small compounds that bind MEF2 may act through at least two mechanisms. One is to block the recruitment of class II HDACs by MEF2. This mechanism will allow the exploration of the clinical benefits of specifically inhibiting class II HDACs without using the broad HDAC inhibitors (see Background). Another mechanism is to block the recruitment of p300 when the MEF2/co-repressor complex is aberrantly disassembled by pathological stimuli in diseased organs such as a hypertrophic heart. The ligand/receptor binding mechanism between MEF2 and its co-regulator (co-repressor and co-activators) indicates that it will be possible to use small molecules to block the recruitment of co-regulators by MEF2.

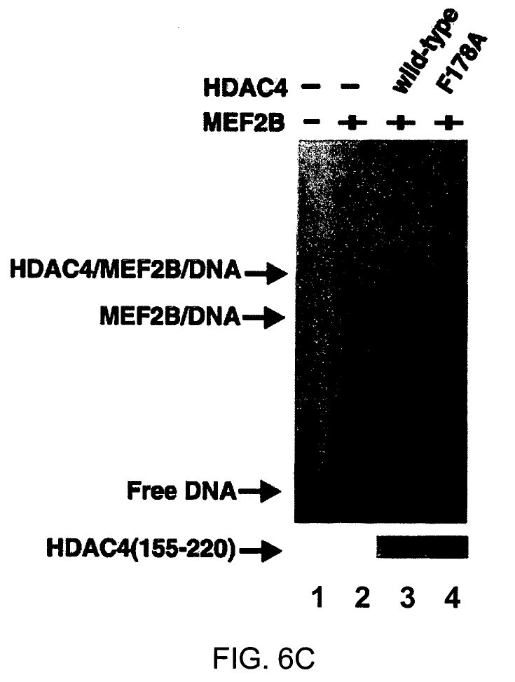

The structure of the HDAC/MEF2/DNA complex reveals that the MEF2 domain forms a helix-strand-helix motif wrapped around the surface of the MADS-box, providing an extensive surface for interactions with MEF2-binding transcription co-regulators. HDAC9 binds MEF2 as an amphipathic alpha helix to form a triple-helix bundle with two helices of the MEF2S domain. Many of the interface residues have been confirmed by the present inventors in mutagenesis studies described herein to be important for HDAC to bind MEF2 in solution (e.g., see Example 2 and FIG. 6). In addition, the present inventors have discovered many other previously unknown sites that are important for the interactions. These sites will be discussed in detail below.

The whole N-terminal conserved MEF2 domain has an architecture with one side for binding DNA and with another for co-repressor, which was observed in the resolution of both complexes. Surprisingly, the inventors found that the ligand groove of MEF2 is actively engaged in binding through adaptive conformational change in response to different ligands. These complex structures and further biochemistry studies suggest a general mechanism by which MEF2 recruits Class II HDACs to repress downstream target genes (discussed below).

Interestingly, the crystal structure of the HDAC9/MEF2/DNA complex reveals protein-binding interactions not seen in the first-derived structure of the related Cabin1/MEF2/DNA complex (discussed in detail below). These new interactions are unique to the subgroup of class II HDACs (HDAC4, HDAC5 and HDAC9) that play critical roles in gene expression associated with heart hypertrophy. This new structural information will be critical for developing, in silico and in vitro, small molecule inhibitors of MEF2 for therapeutic applications in heart hypertrophy. The methods of crystallizing the HDAC9/MEF2/DNA complex will provide an efficient way to characterize MEF2 binding compounds developed by a variety of means such as combinatorial screen and computer-based design, as well as other methods. Thus, the HDAC9/MEF2 structure will be tremendously useful for designing specific inhibitors of MEF2 for human heart diseases.

Indeed, using the information provided by the resolution of the tertiary structures of HDAC9/MEF2/DNA and Cabin1/MEF2/DNA, the present inventors have designed a novel peptide that is a competitive inhibitor of MEF2, competing with the natural cognant ligands of MEF2 such as p300. Such a peptide was designed to bind to MEF2, is water soluble, can efficiently enter the cytoplasm of cells, can distribute to the nucleus, and can modulate MEF2 activity in vitro. Moreover, because the binding affinity of the peptide for MEF2 is less than that of the class II HDACs, the peptide can be used under appropriate dosage and conditions to inhibit MEF2 functions such as MEF2-mediated gene expression during hypertrophic growth of the heart and autoimmune responses of the immune system, but should not affect normal physiological functions of MEF2. Such a peptide is only one example of a variety of such inhibitory peptides and non-peptide compounds that can be developed, designed, or selected using the structures, information, and methods described in the present invention.

Although it was speculated that MEF2 might bind Cabin1 and class II HDACs through a similar mechanism, the structures of the Cabin1/MEF2/DNA complex and the HDAC9/MEF2/DNA complex reveal significant differences in the mechanisms by which MEF2 recruits distinct transcriptional co-repressors. These differences will provide the molecular basis to design compounds of high affinity and specificity that bind MEF2 and block the hypertrophic gene expression. The combined structural information of the Cabin1/MEF2/DNA complex and the HDAC9/MEF2/DNA complex indicate that it is feasible to design small molecule inhibitors and peptide inhibitors for the recruitment of class II HDAC and other co-regulators by MEF2.

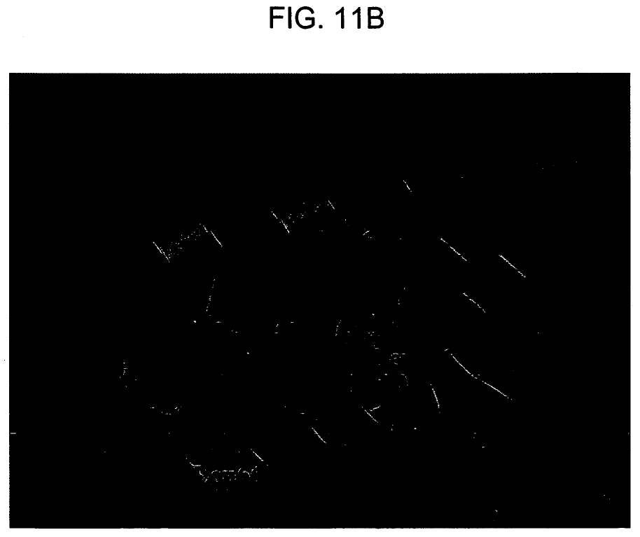

Based on the combined structural information from the complexes described herein, the inventors propose strategies for designing MEF2 inhibitors, including peptide and non-peptide inhibitors (e.g., see FIG. 10 and detailed discussion below). The electrophoresis mobility shift assay (EMSA) developed in the present inventors' studies (described below) will also serve as a high throughput method to screen potential MEF2 inhibitors. The crystallization methods established in the inventors' studies can be used to characterize MEF2 inhibitors generated from computer design and combinatorial screen.

Accordingly, one embodiment of the present invention relates to an isolated peptide that is a regulator (modulator) of MEF2. Preferably, the peptide is an inhibitor of MEF2 activity, although MEF2 stimulators are also encompassed by the invention. Such a peptide differs from a full-length cognant ligand of MEF2 (e.g., an HDAC, Cabin1 or p300) in that it is less than a full-length fragment of such ligands and is preferably less than about 50 amino acids in length, and more preferably less than about 45 amino acids in length, and more preferably less than about 40 amino acids in length, and more preferably less than about 35 amino acids in length, and more preferably less than about 30 amino acids in length, and more preferably less than about 25 amino acids in length, and more preferably less than about 25 amino acids in length, and more preferably less than about 20 amino acids in length, and more preferably less than about 15 amino acids in length. Such a peptide has the properties of being able to specifically (or selectively) bind to MEF2 and to modulate the activity of MEF2 (e.g., by competitively inhibiting the binding of MEF2 by natural ligands or by directly affecting a biological activity of MEF2). Preferably, the peptide also has the characteristics of being water soluble and able to enter or be delivered to the cytoplasm of a cell, and more preferably, is able to enter or be delivered (transported) to the nucleus of a cell. The phrase “selectively binds” refers to the specific binding of one protein to another (e.g., a peptide, a protein, an antibody, a fragment thereof), wherein the level of binding, as measured by any standard assay (e.g., a binding assay), is statistically significantly higher than the background control for the assay. In particular, binding to a target protein should be distinguishable from any non-selective binding to another protein.

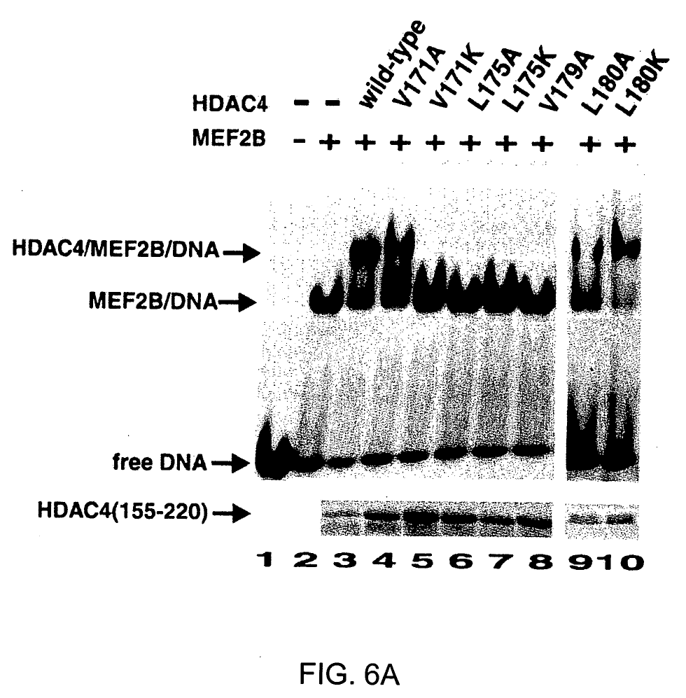

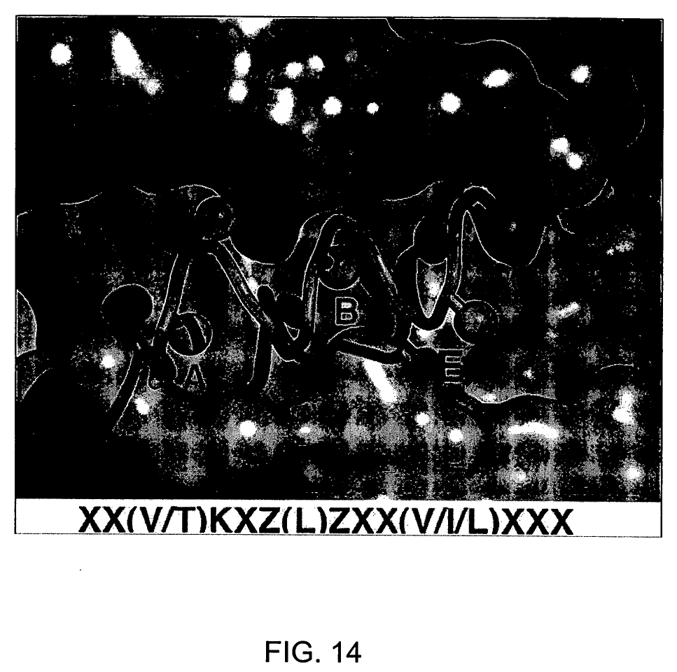

In one aspect of this embodiment, the isolated peptide comprises the amino acid sequence represented by SEQ ID NO:22. As discussed below, SEQ ID NO:22 represents the motif that the present inventors have identified as being particularly characteristic of an amphipathic helix that binds to the concave surface of MEF2, having five discrete hydrophobic pockets (see FIG. 14). This motif denoted: X(V/T)KXZ(L)X(V/T)KXZ(L)ZXX(V/I/L)XX (SEQ ID NO:22). The bracketed single or degenerate amino acids denote the hydrophobic residues inserting into the three hydrophobic pockets on MEF2 and K is a conserved lysine, while X denotes non-conserved amino acids that may or may not interact with MEF2. The two positions sandwiching the central leucine (denoted by letter Z) are often occupied by amino acids with long aliphatic side chains, such as lysine, arginine, or glutamine (FIG. 4). These residues make extensive van der Waals contacts to Thr70 and Leu67 of helix H2 of both MEF2 monomers. Using this motif, one of skill in the art will be able to design a variety of peptides that can be predicted to bind to MEF2 and that may serve as competitive inhibitors of MEF2 function.

In particular embodiments of the invention, the peptide can be a fragment of any of the natural ligands for MEF2 that meet the above criteria, such natural ligands including, but not limited to, the ligands represented by SEQ ID NO:4, SEQ ID NO:6, SEQ ID NO:8, SEQ ID NO:10, SEQ ID NO:12 and SEQ ID NO:14. In one embodiment, the peptide can be a fragment of any homologue of such natural ligands. Homologues are described in detail elsewhere herein. In one aspect, the peptide comprises an amino acid sequence in the first 200-250 N-terminal amino acids of an HDAC. In another aspect, the peptide comprises an amino acid sequence in the first 160 N-terminal amino acid residues of an HDAC. In another embodiment, the peptide comprises an amino acid sequence comprising or aligning with amino acids represented by any one of SEQ ID NO:18, SEQ ID NO:19 or SEQ ID NO:20. In yet another embodiment, the peptide comprises an amino acid sequence comprising or aligning with amino acid residues (with respect to SEQ ID NO:4): Val143, Lys144, Lys146, Leu147, Gln148, Phe150 and Leu151. In yet another embodiment, the peptide comprises an amino acid sequence comprising or aligning with any one or more of amino acids with respect to SEQ ID NO:4: Val143, Lys144, Lys146, Leu147, Gln148, Phe150, Leu151, and Phe177. In yet another embodiment, the peptide comprises an amino acid sequence that binds to a region of MEF2 comprising or aligning with any one or more of amino acids (with respect to SEQ ID NO:2): Gln56, Met62, Asp63, Leu66, Leu67, Tyr69, Thr70, Tyr72, Ser73, Glu74, Pro75, and Ser78.

The present inventors have designed various peptides that are predicted to serve as inhibitors of MEF2 using the structural information provided by the present invention. One exemplary peptide has been produced and tested and indeed is a peptide inhibitor of MEF. This peptide was designed from the HDAC9 amino acid sequence and includes positions 1-19 of SEQ ID NO:23. SEQ ID NO:23 represents a larger chimeric peptide with additional desirable features that have been developed by the present inventors. Specifically, the peptide represented by SEQ ID NO:23 is linked using a peptide linker (positions 20-23 of SEQ ID NO:23) to a transducer peptide (positions 24-36 of SEQ ID NO:23) that effectively transduces the entire chimeric peptide into the nucleus of a cell, where the peptide can inhibit MEF2 by competition with its natural ligands. The transducer peptide used by the inventors is only exemplary, as other similar sequences having similar function (transporter) are known in the art and can be substituted. Therefore, in one embodiment of the invention, a peptide consists essentially of SEQ ID NO:23. Based on the disclosure provided herein, one of skill in the art will be able to envision other peptides that have similar properties, and that can be detectably labeled, for example, and used in in vitro and in vivo methods according to the invention. The present invention also includes pharmaceutical or other compositions comprising such peptides, for use in therapeutic, diagnostic, and/or targeting or discovery assays, or as lead compounds for additional inhibitor development.

According to the present invention, general reference to a protein (e.g., an HDAC protein, a Cabin1 protein, or a MEF2 protein) is reference to a protein that, at a minimum, contains any biologically active portion (e.g., portion with a particular biological function or a portion that at least binds to a given substrate, such as another protein and/or DNA) of the specified protein, and includes full-length proteins, biologically active fragments of the protein, fusion proteins, or any homologue of a naturally occurring protein, as described in detail below. A homologue of a given protein includes proteins which differ from the naturally occurring protein in that at least one or a few, but not limited to one or a few, amino acids have been deleted (e.g., a truncated version of the protein, such as a peptide or fragment), inserted, inverted, substituted and/or derivatized (e.g., by glycosylation, phosphorylation, acetylation, myristoylation, prenylation, palmitation, amidation and/or addition of glycosylphosphatidyl inositol). A homologue can include an agonist of a protein or an antagonist of a protein (described in detail below). Preferably, a homologue of a specified protein has an amino acid sequence that is at least about 30% identical to the amino acid sequence of a naturally occurring protein (e.g., any of the proteins having amino acid sequences disclosed herein, such as SEQ ID NO:2, SEQ ID NO:4, SEQ ID NO:6, SEQ ID NO:8, SEQ ID NO:10, SEQ ID NO:12, SEQ ID NO:14), andmore preferably, at least about 35%, and more preferably, at least about 40%, and more preferably, at least about 45%, and more preferably, at least about 50%, and more preferably, at least about 55%, and more preferably, at least about 60%, and more preferably, at least about 65%, and more preferably, at least about 75%, and more preferably, at least about 75%, and more preferably, at least about 80%, and more preferably, at least about 85%, and more preferably, at least about 90%, and more preferably, at least about 95% identical to the amino acid sequence of a naturally occurring protein, including any increment between 35% and 99%, in whole percentage increments (i.e., 35%, 36%, 37% . . . ). Also included in the invention are any homologues of the following peptide sequences described herein: SEQ ID NO:23 and SEQ ID NO:24.

As used herein, unless otherwise specified, reference to a percent (%) identity refers to an evaluation of homology which is performed using: (1) a BLAST 2.0 Basic BLAST homology search using blastp for amino acid searches and blastn for nucleic acid searches with standard default parameters, wherein the query sequence is filtered for low complexity regions by default (described in Altschul, S. F., Madden, T. L., Schääffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402, incorporated herein by reference in its entirety); (2) a BLAST 2 alignment (using the parameters described below); (3) PSI-BLAST with the standard default parameters (Position-Specific Iterated BLAST; or (4) any of the software programs/algorithms described in the Examples or elsewhere herein. It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST 2 sequence as described in Tatusova and Madden, (1999), “Blast 2 sequences—a new tool for comparing protein and nucleotide sequences”, FEMS Microbiol Lett. 174:247-250, incorporated herein by reference in its entirety. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows:

For blastn, using 0 BLOSUM62 matrix:

Reward for match=1

Penalty for mismatch=−2

Open gap (5) and extension gap (2) penalties

gap x_dropoff (50) expect (10) word size (11) filter (on)

For blastp, using 0 BLOSUM62 matrix:

Open gap (11) and extension gap (1) penalties gap x_dropoff (50) expect (10) word size (3) filter (on).

One of skill in the art can also use any of a number of other software programs that are publicly available. For example, one can use BLOCKS (GIBBS) and MAST (Henikoff et al., 1995, Gene, 163, 17-26; Henikoff et al., 1994, Genomics, 19, 97-107), typically using standard manufacturer defaults.

In one embodiment of the present invention, any of amino acid sequence described herein, as well as homologues of such sequences, can be produced with from at least one, and up to about 20, additional heterologous amino acids flanking each of the C— and/or N-terminal end of the given amino acid sequence. The resulting protein or polypeptide can be referred to as “consisting essentially of” a given amino acid sequence. According to the present invention, the heterologous amino acids are a sequence of amino acids that are not naturally found (i.e., not found in nature, in vivo) flanking the given amino acid sequence or which would not be encoded by the nucleotides that flank the naturally occurring nucleic acid sequence encoding the given amino acid sequence as it occurs in the gene, if such nucleotides in the naturally occurring sequence were translated using standard codon usage for the organism from which the given amino acid sequence is derived. Similarly, the phrase “consisting essentially of”, when used with reference to a nucleic acid sequence herein, refers to a nucleic acid sequence encoding a given amino acid sequence that can be flanked by from at least one, and up to as many as about 60, additional heterologous nucleotides at each of the 5′ and/or the 3′ end of the nucleic acid sequence encoding the given amino acid sequence. The heterologous nucleotides are not naturally found (i.e., not found in nature, in vivo) flanking the nucleic acid sequence encoding the given amino acid sequence as it occurs in the natural gene.

Preferred three-dimensional structural homologues of the proteins of the invention are described in detail below. In one embodiment, a MEF2 homologue has the ability to bind to DNA and/or to a ligand (also referred to herein as a corepressor) of MEF2 (e.g., an HDAC protein, p3000, or any MEF2-binding portions thereof). Such homologues include fragments or mutants of a full length MEF2 and can be referred to herein as a ligand-binding fragment or protein. In one embodiment, a MEF2 homologue has a biological activity of a naturally occurring MEF2 protein. In another embodiment, an HDAC homologue has the ability to bind to a ligand of the HDAC (e.g., MEF2 or any HDAC-binding portions thereof). Such homologues include fragments or mutants of a full length HDAC and can be referred to herein as a ligand-binding fragment or protein. In one embodiment, an HDAC homologue has a biological activity of a naturally occurring HDAC protein. In yet another embodiment, a Cabin1 homologue has the ability to bind to a ligand of Cabin1 (e.g., MEF2 or any Cabin1-binding portions thereof). Such homologues include fragments or mutants of a full length Cabin1 and can be referred to herein as a ligand-binding fragment or protein. In one embodiment, a Cabin1 homologue has a biological activity of a naturally occurring Cabin1 protein.

The minimum size of a protein or domain and/or a homologue or fragment thereof of the present invention is, in one aspect, a size sufficient to have the requisite biological activity, or sufficient to serve as an inhibitor of a MEF2 protein or a cognant ligand thereof, or to serve as an antigen for the generation of an antibody or as a target in an in vitro assay. In one embodiment, a protein of the present invention is at least about 8 amino acids in length, or at least about 25 amino acids in length, or at least about 50 amino acids in length, or at least about 100 amino acids in length, or at least about 150 amino acids in length, or at least about 200 amino acids in length, or at least about 250 amino acids in length, or at least about 300 amino acids in length, or at least about 350 amino acids in length, or at least about 400 amino acids in length, or at least about 450 amino acids in length, or at least about 500 amino acids in length, and so on, in any length between 8 amino acids and up to the full length of a protein or longer, in whole integers (e.g., 8, 9, 10, . . . 25, 26, . . . 500, 501, . . .). There is no limit, other than a practical limit, on the maximum size of such a protein.

According to the present invention, the term “contiguous” or “consecutive”, with regard to nucleic acid or amino acid sequences described herein, means to be connected in an unbroken sequence. For example, for a first sequence to comprise 30 contiguous (or consecutive) amino acids of a second sequence, means that the first sequence includes an unbroken sequence of 30 amino acid residues that is 100% identical to an unbroken sequence of 30 amino acid residues in the second sequence. Similarly, for a first sequence to have “100% identity” with a second sequence means that the first sequence exactly matches the second sequence with no gaps between nucleotides or amino acids.

In general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). Modifications of a protein, such as in a homologue or mimetic (discussed below), may result in proteins having the same biological activity as the naturally occurring protein, or in proteins having decreased or increased biological activity as compared to the naturally occurring protein. Modifications which result in a decrease in protein expression or a decrease in the activity of the protein, can be referred to as inactivation (complete or partial), down-regulation, or decreased action of a protein. Similarly, modifications that result in an increase in protein expression or an increase in the activity of the protein, can be referred to as amplification, overproduction, activation, enhancement, up-regulation or increased action of a protein. As used herein, a protein that has “MEF2 biological activity” or that is referred to as MEF2, by way of example, refers to a protein that has an activity that can include any one, and preferably more than one, of the following characteristics: (a) interacts with (e.g., by binding to) a substrate or ligand of a naturally occurring MEF2 protein or close variant thereof (e.g., DNA, an HDAC, p300, and/or other substrate or fragment thereof); (b) functional activity, such as contributing to transcriptional repression, contributing to calcium-dependent de-repression; or contributing to transcription activation, which result in phenotypes such as regulation of the expression of specific genes involved in the development and adaptive responses of muscle cells, neuronal cells, T cells, or other characterized downstream effects.

Similarly a protein that has “Cabin1 biological activity” or that is referred to as Cabin1, by way of example, refers to a protein that has an activity that can include any one, and preferably more than one, of the following characteristics: (a) interacts with (e.g., by binding to) a substrate or ligand of a naturally occurring Cabin1 protein or close variant thereof (e.g., MEF2 and/or other substrate or fragment thereof); (b) functional activity, such as contributing to transcriptional repression, which results in phenotypes such as regulation of the expression of specific genes involved in the development and adaptive responses of T cells, or other characterized downstream effects.

A protein that has “HDAC biological activity” or that is referred to as an HDAC, by way of example, refers to a protein that has an activity that can include any one, and preferably more than one, of the following characteristics: (a) interacts with (e.g., by binding to) a substrate or ligand of a naturally occurring HDAC protein or close variant thereof (e.g., MEF2 and/or other substrate or fragment thereof); (b) functional activity, such as contributing to transcriptional repression, which results in phenotypes such as regulation of the expression of specific genes involved in the development and adaptive responses of muscle cells, or other characterized downstream effects. The specific biological activities of a variety of HDAC proteins, including the class II HDAC proteins (e.g., HDAC4, HDAC5, HDAC7 and HDAC9) are well known in the art.

The nucleic acid and amino acid sequences of representative members of the class II HDAC family are provided herein. For example, as discussed above, amino acid sequence for the murine HDAC9 isoform known as MEF2 interacting transcriptional repressor (MITR) (GenBank Accession No. NM_{—}024124) is represented herein by SEQ ID NO:4. SEQ ID NO:4 is encoded by the nucleic acid sequence of SEQ ID NO:3. The amino acid sequence of the human HDAC9, splice variant 1 (GenBank Accession No. NM_{—}058176), is represented herein by SEQ ID NO:6, encoded by SEQ ID NO:5. The amino acid sequence of the human HDAC4 (GenBank Accession No. NM_{—}006037) is represented herein by SEQ ID NO:8, encoded by SEQ ID NO:7. The amino acid sequence of the human HDAC5, splice variant 1 (GenBank Accession No. NM_{—}005474) is represented herein by SEQ ID NO:10, encoded by SEQ ID NO:9. The amino acid sequence of the human HDAC7a (GenBank Accession No. BC006453) is represented herein by SEQ ID NO:12, encoded by SEQ ID NO:11. Other splice variants/isoforms of the class II HDAC family members and their nucleotide and amino acid sequences are well known in the art and are intended to be encompassed by the present invention.

An isolated protein, according to the present invention, is a protein that has been removed from its natural milieu (i.e., that has been subject to human manipulation) and can include purified proteins, partially purified proteins, recombinantly produced proteins, and synthetically produced proteins, for example. As such, “isolated” does not reflect the extent to which the protein has been purified. Preferably, an isolated protein, and particularly (including fragments and homologues thereof), is produced recombinantly or synthetically (if the protein is a smaller peptide). The terms “fragment”, “segment” and “portion” can be used interchangeably herein with regard to referencing a part of a protein. It will be appreciated that, as a result of the determination of the tertiary structure of MEF2 and MEF2 corepressors herein, various portions of these proteins will now be appreciated as being particularly valuable for mutational analyses and various biological assays outside of the computer-assisted drug design methods disclosed herein. In addition, such portions of proteins will be particularly valuable for use in the design and development of new regulators of MEF2 activity. Such portions of these proteins, including MEF2, an HDAC protein (and particularly a class II HDAC protein, and more particularly, HDAC 9 or MITR), and Cabin1, and methods of using such portions are explicitly contemplated to be part of the present invention.

Reference to a protein from a specific organism, such as a “human HDAC”, by way of example, refers to an HDAC (including a homologue of a naturally occurring HDAC) from a Homo sapiens or to an HDAC that has been otherwise produced from the knowledge of the primary structure (e.g., sequence) and/or the tertiary structure of a naturally occurring HDAC from Homo sapiens. In other words, a human HDAC includes any HDAC that has the structure and function of a naturally occurring HDAC from Homo sapiens or that has a structure and function that is sufficiently similar to a Homo sapiens HDAC such that the HDAC is a biologically active (i.e., has biological activity) homologue of a naturally occurring HDAC from Homo sapiens. As such, a Homo sapiens HDAC, by way of example, can include purified, partially purified, recombinant, mutated/modified and synthetic proteins.

Proteins and peptides may be produced recombinantly or may be synthesized. For smaller peptides, chemical synthesis methods may be preferred. For example, such methods include well known chemical procedures, such as solution or solid-phase peptide synthesis, or semi-synthesis in solution beginning with protein fragments coupled through conventional solution methods. Such methods are well known in the art and may be found in general texts and articles in the area such as: Merrifield, 1997, Methods Enzymol. 289:3-13; Wade et al., 1993, Australas Biotechnol. 3(6):332-336; Wong et al., 1991, Experientia 47(11-12):1123-1129; Carey et al., 1991, Ciba Found Symp. 158:187-203; Plaue et al., 1990, Biologicals 18(3):147-157; Bodanszky, 1985, Int. J. Pept. Protein Res. 25(5):449-474; or H. Dugas and C. Penney, BIOORGANIC CHEMISTRY, (1981) at pages 54-92, all of which are incorporated herein by reference in their entirety.

Proteins of the present invention are preferably retrieved, obtained, and/or used in “substantially pure” form. As used herein, “substantially pure” refers to a purity that allows for the effective use of the protein in vitro, ex vivo or in vivo according to the present invention. For a protein to be useful in an in vitro, ex vivo or in vivo method according to the present invention, it is substantially free of contaminants, other proteins and/or chemicals that might interfere or that would interfere with its use in a method disclosed by the present invention, or that at least would be undesirable for inclusion with the protein when it is used in a method disclosed by the present invention. For example, for an HDAC protein, such methods include crystallization of the protein, use of all or a portion of the protein for mutational analysis, for antibody production, for agonist/antagonist identification assays, and all other methods disclosed herein. For a therapeutic or lead compound peptide or protein identified by the structure-based identification methods described herein, such methods include synthesis of the protein or peptide, collection or recovery of the protein or peptide, use of the protein or peptide in high-throughput screening assays, and therapeutic development and use of the protein or peptide. Preferably, a “substantially pure” protein, as referenced herein, is a protein that can be produced by any method (i.e., by direct purification from a natural source, recombinantly, or synthetically), and that has been purified from other protein components such that the protein comprises at least about 80% weight/weight of the total protein in a given composition (e.g., the protein is about 80% of the protein in a solution/composition/buffer), and more preferably, at least about 85%, and more preferably at least about 90%, and more preferably at least about 91%, and more preferably at least about 92%, and more preferably at least about 93%, and more preferably at least about 94%, and more preferably at least about 95%, and more preferably at least about 96%, and more preferably at least about 97%, and more preferably at least about 98%, and more preferably at least about 99%, weight/weight of the total protein in a given composition.

As used herein, a “structure” of a protein refers to the components and the manner of arrangement of the components to constitute the protein. The “three dimensional structure” or “tertiary structure” of the protein refers to the arrangement of the components of the protein in three dimensions. Such term is well known to those of skill in the art. It is also to be noted that the terms “tertiary” and “three dimensional” and “ternary” can be used interchangeably.

The present invention provides the atomic coordinates that define the three dimensional structure of a Cabin1/MEF2/DNA complex, an HDAC9(MITR)/MEF2/DNA complex, and accordingly, the atomic coordinates that define the three dimensional structures of the individual components as they occur when in complex with one another. First, the present inventors have determined the atomic coordinates that define the three dimensional structure of a crystalline Cabin1/MEF2B/DNA complex (see Example 1 for details). Second, the present inventors have determined the atomic coordinates that define the three dimensional structure of a crystalline HDAC9(MITR)/MEF2B/DNA complex (see Example 2 for details). Using the guidance provided herein, one of skill in the art will be able to reproduce any of such structures, including the structures of the individual components therein (e.g., MEF2B, Cabin1, or HDAC9(MITR)) and define atomic coordinates of such a structure.

Example 1 describes the production of a Cabin1/MEF2B/DNA complex, arranged in a crystalline manner in a space group P4₁22 so as to form a unit cell of dimensions a=b=70.14 Å and c=151.88 Å. The complex was produced from a fusion protein comprising human MEF2B (residues 1-93 of SEQ ID NO:2) and human Cabin1 (residues 2156-2190 of SEQ ID NO:14) with Cabin1 at the C-terminus and a PreScission site in between. A 20% molar excess of DNA was mixed with the Cabin1/MEF2B fusion protein to produce the complex. The atomic coordinates determined from this crystal structure and defining the three dimensional structure of the complex are provided as Table 2 of PCT Application No. PCT/US04/011744, which published as PCT Publication No. WO 2004/094591 on Nov. 4, 2004, the entire disclosure of which is incorporated herein by reference. The atomic coordinates for the Cabin1/MEF2B/DNA complex (Table 2 of PCT Application No. PCT/US04/011744) were deposited with the Protein Data Bank (PDB), operated by the Research Collaboratory for Structural Bioinformatics (RCSB) (H. M. Berman, J. Westbrook, Z. Feng, G. Gilliland, T. N. Bhat, H. Weissig, I. N. Shindyalov, P. E. Bourne, The Protein Data Bank; Nucleic Acids Research, 28:235-242 (2000)), under PDB Deposit No. 1N6J on Nov. 11, 2002 and released to the public on Nov. 11, 2003, and such coordinates are incorporated herein by reference in entirety.

The atomic coordinates for the individual components of the complex (Cabin1, MEF2B and DNA) can be identified by the unique chain identification for each component (Chain ID) and thus may be separately used in the methods of the invention. For the purposes of this invention, reference to the complex defined by the atomic coordinates in PDB Deposit No. 1N6J or Table 2 of PCT Application No. PCT/US04/011744 can be made by referring to a Cabin1/MEF2B/DNA complex or a Cabin1/MEF2/DNA complex, or any other arrangement of the three components (e.g., MEF2B/Cabin1/DNA). Reference to the atomic coordinates in PDB Deposit No. 1N6J or the atomic coordinates represented in Table 2 of PCT Application No. PCT/US04/011744 can be used interchangeably.

Example 2 describes the production of an HDAC9 (MITR isoform)/MEF2B/DNA complex arranged in a crystalline manner in a space group P1 so as to form a unit cell of dimensions a=44.765 Å, b=66.859 Å, c=66.924 Å (alpha=76.656, beta=71.846, gamma=71.799). The complex was produced from a fusion protein comprising human MEF2B (residues 1-93 of SEQ ID NO:2) and murine MITR (residues 128-154 of SEQ ID NO:4) with MITR at the C-terminus. A 20% molar excess of DNA was mixed with the MITR/MEF2 fusion protein to produce the complex. The atomic coordinates defining this crystal structure are provided as Table 1 of PCT Application No. PCT/US04/011744, which published as PCT Publication No. WO 2004/094591 on Nov. 4, 2004, the entire disclosure of which is incorporated herein by reference. The atomic coordinates for the HDAC9/MEF2/DNA ternary complex (Table 1 of PCT Application No. PCT/US04/011744) were deposited with the Protein Data Bank (PDB), operated by the Research Collaboratory for Structural Bioinformatics (RCSB) (H. M. Berman, J. Westbrook, Z. Feng, G. Gilliland, T. N. Bhat, H. Weissig, I. N. Shindyalov, P. E. Bourne, The Protein Data Bank; Nucleic Acids Research, 28:235-242 (2000)), under PDB Deposit No. 1TQE on Jun. 17, 2004 and released to the public on Dec. 21, 2004, and such coordinates are incorporated herein by reference in entirety.

The atomic coordinates for the individual components of the complex (MITR, MEF2B and DNA) can be identified by the unique chain identification for each component (Chain ID) and thus may be separately used in the methods of the invention. For the purposes of this invention, reference to the complex defined by the atomic coordinates in PDB Deposit No. ITQE or Table 1 of PCT Application No. PCT/US04/011744 can be made by referring to an HDAC/MEF2/DNA complex (or MEF2 can be designated MEF2B), an HDAC9/MEF2/DNA complex (or MEF2 can be designated MEF2B) or a MITR/MEF2/DNA complex (or MEF2 can be designated MEF2B), or any other arrangement of the three components (e.g., MEF2B/MITR/DNA). Reference to the atomic coordinates in PDB Deposit No. ITQE or the atomic coordinates represented in Table 1 of PCT Application No. PCT/US04/011744 can be used interchangeably.

One embodiment of the present invention includes any of the above-described complexes in crystalline form. The present invention specifically exemplifies crystalline Cabin1/MEF2/DNA and crystalline MITR/MEF2/DNA. As used herein, by way of example, the terms “crystalline MITR/MEF2/DNA” and “MITR/MEF2/DNA crystal” both refer to crystallized MITR/MEF2/DNA complex and are intended to be used interchangeably. Preferably, a crystalline complex of the invention is produced using the crystal formation method described herein, in particular according to the method disclosed in Example 1 or Example 2. A MITR/MEF2/DNA crystal or a Cabin1/MEF2/DNA crystal of the present invention can comprise any crystal structure that comes from crystals formed in any of the allowable space groups for these complexes proteins. In one aspect, a crystalline MITR/MEF2/DNA of the present invention includes MITR/MEF2/DNA molecules arranged in a crystalline manner in a space group P1 of the crystal lattice so as to form a unit cell having approximate dimensions of P1 so as to form a unit cell of dimensions a=44.765 Å, b=66.859 Å, c=66.924 Å (alpha=76.656, beta=71.846, gamma=71.799). In one aspect, a crystalline Cabin1/MEF2/DNA of the present invention includes Cabin1/MEF2/DNA molecules arranged in a crystalline manner in a space group of P4₁22 of the crystal lattice so as to form a unit cell of dimensions a=b=70.14 Å and c=151.88 Å.

According to the present invention, a unit cell having “approximate dimensions of” a given set of dimensions refers to a unit cell that has dimensions that are within plus (+) or minus (−) 2.0% of the specified unit cell dimensions. Such a small variation is within the scope of the invention since one of skill in the art could obtain such variance by performing X-ray crystallography at different times on the same crystal. In one embodiment, a crystalline complex of the present invention has the specified unit cell dimensions set forth above. A preferred crystal of the present invention provides X-ray diffraction data for determination of atomic coordinates of the complex to a resolution of about 4.0 Å, and preferably to about 3.2 Å, and preferably to about 3.0 Å, and more preferably to about 2.3 Å, and more preferably to about 2.0 Å, and even more preferably to about 1.8 Å.

One embodiment of the present invention includes a method for producing crystals of a complex of the invention, including MITR/MEF2/DNA and Cabin1/MEF2/DNA, comprising combining the complex with a mother liquor and inducing crystal formation to produce the complex crystals. Although the production of crystals of two different complexes are specifically described herein, it is to be understood that such processes as are described herein can be adapted by those of skill in the art to produce crystals of other MEF2/MEF2 ligand/DNA complexes. A suitable mother liquor of the present invention comprises the solution used for crystallization as described in Examples 1 or 2 that causes the protein complex to crystallize. There is some tolerance in the mother liquor conditions so that changes of up to 30% in buffer concentrations, PEG concentrations, 0.5 pH units, and temperatures of between 10° C. and 28° C. can still yield crystals. Supersaturated solutions comprising a complex can be induced to crystallize by several methods including, but not limited to, vapor diffusion, liquid diffusion, batch crystallization, constant temperature and temperature induction or a combination thereof. Preferably, supersaturated solutions of the complex are induced to crystallize by hanging drop vapor diffusion. In a vapor diffusion method, a complex is combined with a mother liquor as described above that will cause the protein solution to become supersaturated and form crystals at a constant temperature. Vapor diffusion is preferably performed under a controlled temperature and, by way of example, can be performed at 18° C.

The crystalline complexes of the present invention are analyzed by X-ray diffraction and, based on data collected from this procedure, models are constructed which represent the tertiary structure of the complexes. Therefore, one embodiment of the present invention includes a representation, or model, of the three dimensional structure of a complex of the present invention or of a component thereof (e.g., MEF2, Cabin1, HDAC), such as a computer model. A computer model of the present invention can be produced using any suitable software modeling program, including, but not limited to, the graphical display program O (Jones et. al., Acta Crystallography, vol. A47, p. 110, 1991), CNS (Brunger, et al. Crystallography & NMR system: A new software suite for macromolecular structure determination. Acta Crystallogr D Biol Crystallogr 54, 905-21. (1998)), the graphical display program GRASP, MOLSCRIPT 2.0 (Avatar Software AB, Heleneborgsgatan 21C, SE-11731 Stockholm, Sweden), the program CONTACTS from the CCP4 suite of programs (Bailey, 1994, Acta Cryst. D50:760-763), or the graphical display program INSIGHT. Suitable computer hardware useful for producing an image of the present invention are known to those of skill in the art (e.g., a Silicon Graphics Workstation).

A representation, or model, of the three dimensional structure of the complex or protein component for which a crystal has been produced can also be determined using techniques which include molecular replacement or SIR/MIR (single/multiple isomorphous replacement), or MAD (multiple wavelength anomalous diffraction) methods (Hendrickson et al., 1997, Methods Enzymol., 276:494-522). Methods of molecular replacement are generally known by those of skill in the art (generally described in Brunger, Meth. Enzym., vol. 276, pp. 558-580, 1997; Navaza and Saludjian, Meth. Enzym., vol. 276, pp. 581-594, 1997; Tong and Rossmann, Meth. Enzym., vol. 276, pp. 594-611, 1997; and Bentley, Meth. Enzym., vol. 276, pp. 611-619, 1997, each of which are incorporated by this reference herein in their entirety) and are performed in a software program including, for example, AmoRe (CCP4, Acta Cryst. D50, 760-763 (1994), SOLVE (Terwilliger et al., 1999, Acta Crystallogr., D55:849-861), RESOLVE (Terwilliger, 2000, Acta Crystallogr., D56:965-972) or XPLOR. Briefly, X-ray diffraction data is collected from the crystal of a crystallized target structure. The X-ray diffraction data is transformed to calculate a Patterson function. The Patterson function of the crystallized target structure is compared with a Patterson function calculated from a known structure (referred to herein as a search structure). The Patterson function of the crystallized target structure is rotated on the search structure Patterson function to determine the correct orientation of the crystallized target structure in the crystal. The translation function is then calculated to determine the location of the target structure with respect to the crystal axes. Once the crystallized target structure has been correctly positioned in the unit cell, initial phases for the experimental data can be calculated. These phases are necessary for calculation of an electron density map from which structural differences can be observed and for refinement of the structure. Preferably, the structural features (e.g., amino acid sequence, conserved disulphide bonds, and β-strands or β-sheets) of the search molecule are related to the crystallized target structure.

As used herein, the term “model” refers to a representation in a tangible medium of the three dimensional structure of a protein, polypeptide or peptide. For example, a model can be a representation of the three dimensional structure in an electronic file, on a computer screen, on a piece of paper (i.e., on a two dimensional medium), and/or as a ball-and-stick figure. Physical three-dimensional models are tangible and include, but are not limited to, stick models and space-filling models. The phrase “imaging the model on a computer screen” refers to the ability to express (or represent) and manipulate the model on a computer screen using appropriate computer hardware and software technology known to those skilled in the art. Such technology is available from a variety of sources including, for example, Evans and Sutherland, Salt Lake City, Utah, and Biosym Technologies, San Diego, Calif. The phrase “providing a picture of the model” refers to the ability to generate a “hard copy” of the model. Hard copies include both motion and still pictures. Computer screen images and pictures of the model can be visualized in a number of formats including space-filling representations, a carbon traces, ribbon diagrams and electron density maps. A variety of such representations of the structural models of the present invention are shown, for example, in the figures.

Preferably, a three dimensional structure of a complex or component thereof provided by the present invention includes:

(a) a structure defined by atomic coordinates of a three dimensional structure of a crystalline MEF2 region in complex with DNA and a protein selected from: Cabin1 or HDAC9 (MITR);

(b) a structure defined by atomic coordinates selected from:
(i) atomic coordinates represented by a PDB Accession No. selected from PDB Accession No. 1TQE (HDAC9/MEF2/DNA) and PDB Accession No. 1N6J (Cabin1/MEF2/DNA); and,
(ii) atomic coordinates that define a three dimensional structure wherein at least 50% of the structure has an average root-mean-square deviation (RMSD) from backbone atoms in secondary structure elements in at least one domain of a three dimensional structure represented by the atomic coordinates of (1) of equal to or less than about 1.5 Å;

(c) a structure defined by atomic coordinates derived from HDAC9/MEF2/DNA molecules arranged in a crystalline manner in a space group P1 so as to form a unit cell of dimensions a=44.765 Å, b=66.859 Å, c=66.924 Å (alpha=76.656, beta=71.846, gamma=71.799);

(d) a structure defined by atomic coordinates derived from Cabin1/MEF2/DNA molecules arranged in a crystalline manner in a space group P4₁22 so as to form a unit cell of dimensions a=b=70.14 Å and c=151.88 Å; or

(e) a structure of MEF2 in complex with an HDAC protein and DNA constructed using as a template the three-dimensional structure of (ii).

The crystalline complexes, including crystalline Cabin1/MEF2/DNA or crystalline HDAC/MEF2/DNA, have been described in detail above, as well as methods to produce, analyze and model the structure of such crystals (see also Examples 1 and 2). In addition, the atomic coordinates represented in PDB Accession Nos. 1TQE or 1N6J, which define the tertiary structures of the complexes and components thereof have also been discussed above (see also Examples 1 and 2).

In one aspect of the invention, a three dimensional structure of a complex or a component thereof provided by the present invention includes a structure wherein the structure has an average root-mean-square deviation (RMSD) of equal to or less than about 1.7 Å over the backbone atoms in secondary structure elements of at least 50% of the residues in at least one domain of a three dimensional structure represented by the atomic coordinates of any one of PDB Accession Nos. 1TQE or 1N6J. Such a structure can be referred to as a structural homologue of the complexes or components thereof defined by one of PDB Accession Nos. 1TQE or 1N6J. Preferably, the structure has an average root-mean-square deviation (RMSD) of equal to or less than about 1.6 Å over the backbone atoms in secondary structure elements of at least 50% of the residues in at least one domain of a three dimensional structure represented by the atomic coordinates of any one of PDB Accession Nos. 1TQE or 1N6J, or equal to or less than about 1.5 Å, or equal to or less than about 1.4 Å, or equal to or less than about 1.3 Å, or equal to or less than about 1.2 Å, or equal to or less than about 1.1 Å, or equal to or less than about 1.0 Å, or equal to or less than about 0.9 Å, or equal to or less than about 0.8 Å, or equal to or less than about 0.7 Å, or equal to or less than about 0.6 Å, or equal to or less than about 0.5 Å, or equal to or less than about 0.4 Å, or equal to or less than about 0.3 Å, or equal to or less than about 0.2 Å, over the backbone atoms in secondary structure elements of at least 50% of the residues in at least one domain of a three dimensional structure represented by the atomic coordinates of any one of PDB Accession Nos. 1TQE or 1N6J. In another aspect, a three dimensional structure of a complex or component thereof provided by the present invention includes a structure wherein the structure has the recited RMSD over the backbone atoms in secondary structure elements of at least 75% of the residues in at least one domain of a three dimensional structure represented by the atomic coordinates of any one of PDB Accession Nos. 1TQE or 1N6J, and more preferably at least about 80%, and more preferably at least about 85%, and more preferably at least about 90%, and more preferably at least about 95%, and most preferably, about 100% of the residues in at least one domain of a three dimensional structure represented by the atomic coordinates of any one of PDB Accession Nos. 1TQE or 1N6J.

In one embodiment, the RMSD of a structural homologue of a complex or component thereof can be extended to include atoms of amino acid side chains. As used herein, the phrase “common amino acid side chains” refers to amino acid side chains that are common to both the structural homologue and to the structure that is actually represented by such atomic coordinates (e.g., a structure represented by one of PDB Accession Nos. 1TQE or 1N6J). Preferably, at least 50% of the structure has an average root-mean-square deviation (RMSD) from common amino acid side chains in a three dimensional structure represented by the atomic coordinates of one of PDB Accession Nos. 1TQE or 1N6J of equal to or less than about 1.7 Å, or equal to or less than about 1.6 Å, equal to or less than about 1.5 Å, or equal to or less than about 1.4 Å, or equal to or less than about 1.3 Å, or equal to or less than about 1.2 Å, or equal to or less than about 1.1 Å, or equal to or less than about 1.0 Å, or equal to or less than about 0.9 Å, or equal to or less than about 0.8 Å, or equal to or less than about 0.7 Å, or equal to or less than about 0.6 Å, or equal to or less than about 0.5 Å, or equal to or less than about 0.4 Å, or equal to or less than about 0.3 Å, or equal to or less than about 0.2 Å. In another embodiment, a three dimensional structure of a complex or component thereof provided by the present invention includes a structure wherein at least about 75% of such structure has the recited average root-mean-square deviation (RMSD) value, and more preferably, at least about 85% of such structure has the recited average root-mean-square deviation (RMSD) value, and most preferably, about 95% of such structure has the recited average root-mean-square deviation (RMSD) value.

In addition to having the recited RMSD values, a structural homologue of a MEF2-ligand-DNA complex or component thereof should additionally meet the following criteria for amino acid sequence homology, both of which have been discussed in detail previously herein. First, the structure should include or represent a protein having an amino acid sequence corresponding to a relevant homologue of MEF2, HDAC and/or Cabin1 (e.g., has at least one biological activity of the natural protein, including a binding activity or a functional activity). For example, although the HDAC9 isoform known as MITR was used in one of the complexes described herein, other class II HDAC proteins have conserved regions that can result in such protein being considered to be homologues of MITR that can be modeled using as a template the three-dimensional structure of MITR as described in detail herein.

Another structure that is useful in the methods of the present invention is a structure that is defined by the atomic coordinates in any one of PDB Accession Nos. 1TQE or 1N6J defining a component or a portion of a component in an HDAC/MEF2/DNA complex or a Cabin1/MEF2/DNA complex, wherein the portion of the complex or component thereof comprises sufficient structural information to perform structure based drug design (described below). Suitable portions of the complex and components thereof that could be modeled and used in structure based drug design will be apparent to those of skill in the art. For example, one can model all or a portion of the entire complex or an active site thereof, or all or a portion of a component of the complex (e.g., HDAC (MITR), Cabin1, MEF2B). The present inventors have also identified multiple sites of interest based on the structure of the complexes of the invention (described in detail below). Structures comprising these portions (e.g., the ligand binding groove formed by a MEF2 dimer and/or the amphipathic helix of HDAC or Cabin1 that binds within this MEF2 groove; e.g., see FIG. 4 for example) would be encompassed by the present invention.

Accordingly, one embodiment of the present invention relates to a method of structure-based identification of compounds that regulate the interactions of myocyte enhancer factor 2 (MEF2) with its cognate ligands (e.g., an HDAC, such as MITR; Cabin1; or even another ligand such as p300). Such compounds can regulate the ability of either MEF2 or its ligand to bind to one another and/or the biological activity of the either MEF2 or its ligand, such as the transcriptional regulatory activity of the protein. The method is typically a computer-assisted method of structure based drug design, and includes the steps of: (a) providing atomic coordinates that define the three dimensional structure of an HDAC/MEF2/DNA complex or component thereof or a Cabin1/MEF2/DNA complex or component thereof, including a model of a component that uses as a template the actual atomic coordinates provided herein, and including any of the three dimensional structures or atomic coordinates described herein; and (b) identifying at least one candidate compound for interacting with the three dimensional structure of an active site in MEF2, HDAC, Cabin1, an HDAC and MEF2 complex, or a Cabin1 and MEF2 complex by performing structure based drug design with the structure of (a). The step of identifying is typically performed in conjunction with computer modeling.

According to the present invention, a “cognate ligand” of a MEF2 protein is any protein that interacts with or more particularly, binds to, a MEF2 protein in nature (e.g., under any normal, natural, or physiological conditions in vitro or in vivo). As such, the term “cognate” is intended to refer to the relationship in nature between MEF2 and other ligands. The term ligand is intended to generically or generally refer to any ligand, binding partner, corepressor, substrate (such terms being capable of use interchangeably) or other protein or compound with which MEF2 interacts. As such, the term implies any interaction relationship between MEF2 and another compound.

The structures and atomic coordinates used to perform the above-described method have been described in detail above and in the Examples section, and include any structural homologues of proteins described herein. According to the present invention, the phrase “providing atomic coordinates that define the three dimensional structure” is defined as any means of obtaining, providing, supplying, accessing, displaying, retrieving, or otherwise making available the atomic coordinates defining any three dimensional structures as described herein. For example, the step of providing can include, but is not limited to, accessing the atomic coordinates for the structure from a database or other source; importing the atomic coordinates for the structure into a computer or other database; displaying the atomic coordinates and/or a model of the structure in any manner, such as on a computer, on paper, etc.; and determining the three dimensional structure described by the present invention de novo using the guidance provided herein.

The second step of the method of structure based identification of compounds of the present invention includes identifying a candidate compound for interacting with an active site in MEF2, HDAC, Cabin1, an HDAC and MEF2 complex, or a Cabin1 and MEF2 complex, represented by the structure model, by performing structure based drug design with the model of the structure. According to the present invention, the step of “identifying” can refer to any screening process, modeling process, design process, or other process by which a compound can be selected as useful for binding or inhibiting the activity of protein or complex according to the present invention. Methods of structure-based identification of compounds are described in detail below. As discussed above, the interaction of MEF2 with its cognate or natural ligands (e.g., corepressors such as HDACs and Cabin1 or proteins such as p300) regulate the expression and activity of a variety of genes involved in the development and adaptive responses of a variety of cells, including muscle cells, neuronal cells, and T cells. Therefore, the selection of compounds that compete with, disrupt or otherwise inhibit the biological activity of such complexes, or alternatively that enhance, activate or otherwise stimulate the biological activity of such complexes are highly desirable. Such compounds can be designed using structure based drug design using models of the structures disclosed herein. Until the discovery of the three dimensional structures of the complexes of the present invention, the only information available for the development of therapeutic compounds based was based on the primary sequence of the components and mutagenesis studies directed to the isolated protein, and the tertiary structure of MEF2A in complex with DNA, but without the interaction of the cognate ligands (Cabin1 and HDAC (MITR)) described herein.

Structure based identification of compounds (e.g., structure based drug design, structure based compound screening, or structure based structure modeling) refers to the prediction or design of a conformation of a peptide, polypeptide, protein, or to the prediction or design of a conformational interaction between such protein, peptide or polypeptide, and a candidate compound, by using the three dimensional structure of the peptide, polypeptide or protein. Typically, structure based identification of compounds is performed with a computer (e.g., computer-assisted drug design, screening or modeling). For example, generally, for a protein to effectively interact with (e.g., bind to) a compound, it is necessary that the three dimensional structure of the compound assume a compatible conformation that allows the compound to bind to the protein in such a manner that a desired result is obtained upon binding. Knowledge of the three dimensional structure of the components of the complexes described herein in the conformation in which they bind to one another enables a skilled artisan to design a compound having such compatible conformation, or to select such a compound from available libraries of compounds and/or structures thereof. For example, knowledge of the three dimensional structure of the ligand binding groove of MEF2 dimers or of the differing placement of the amphipathic helices of Cabin1 and HDAC within this groove enables one of skill in the art to design or select a compound structure that is predicted to bind to MEF2 or the ligand at that site and result in, for example, inhibition of the binding of MEF2 to its natural ligand or inhibition of the binding of HDAC, Cabin 1, or another MEF2 ligand (e.g., p300) to a natural MEF2 protein. Thereby, one can inhibit a biological response such as repression of gene transcription. Similarly, one can design or select (identify) a compound that has the opposite, or stimulatory effect on the complex components. In addition, for example, knowledge of the three dimensional structure of the proteins and complex herein enables a skilled artisan to design an analog of, for example, Cabin1, HDAC or MEF2.

Suitable structures and models useful for structure based drug design are disclosed herein. Preferred target structures to use in a method of structure based drug design include any representations of structures produced by any modeling method disclosed herein, including molecular replacement and fold recognition related methods.

According to the present invention, the step of identifying, selecting or designing a compound for testing in a method of structure based identification of the present invention can include creating a new chemical compound structure or searching databases of libraries of known compounds (e.g., a compound listed in a computational screening database containing three dimensional structures of known compounds). Designing can also be performed by simulating chemical compounds having substitute moieties at certain structural features. The step of designing can include selecting a chemical compound based on a known function of the compound. Chemical compounds can generally include any peptide, oligonucleotide, carbohydrate and/or synthetic organic molecule. A preferred step of designing comprises computational screening of one or more databases of compounds in which the three dimensional structure of the compound is known and is interacted (e.g., docked, aligned, matched, interfaced) with the three dimensional structure of a complex of the invention (or protein or DNA component thereof) by computer (e.g. as described by Humblet and Dunbar, Animal Reports in Medicinal Chemistry, vol. 28,pp. 275-283, 1993, M Venuti, ed., Academic Press). The compound itself, if identified as a suitable candidate by the method of the invention, can be synthesized and tested directly with one or more of the components in a MEF2-MEF2 ligand complex, and additionally with DNA, for example, in a biological assay. Methods to synthesize suitable chemical or protein-based compounds are known to those of skill in the art and depend upon the structure of the chemical being synthesized. Such methods are discussed in detail below. Methods to evaluate the bioactivity of the synthesized compound depend upon the bioactivity of the compound (e.g., inhibitory or stimulatory) and are discussed herein.

Various other methods of structure-based drug design are disclosed in Maulik et al., 1997, Molecular Biotechnology: Therapeutic Applications and Strategies, Wiley-Liss, Inc., which is incorporated herein by reference in its entirety. Maulik et al. disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

In a molecular diversity strategy, large compound libraries are synthesized, for example, from peptides, oligonucleotides, carbohydrates and/or synthetic organic molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands for a desired target, and then to optimize the lead molecules by either random or directed design strategies. Methods of molecular diversity are described in detail in Maulik, et al., ibid.

Maulik et al. also disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

In the present method of structure based identification of compounds, it is not necessary to align the structure of a candidate chemical compound (i.e., a chemical compound being analyzed in, for example, a computational screening method of the present invention) to each residue in a target site (target sites will be discussed in detail below). Suitable candidate chemical compounds can align to a subset of residues described for a target site. Preferably, a candidate chemical compound comprises a conformation that promotes the formation of covalent or noncovalent crosslinking between the target site and the candidate chemical compound. In one aspect, a candidate chemical compound binds to a surface adjacent to a target site to provide an additional site of interaction in a complex. When designing an antagonist (e.g., a chemical compound that inhibits the biological activity of a MEF2 protein or an HDAC protein), for example, the antagonist should bind with sufficient affinity to the target binding site or substantially prohibit a ligand (e.g., a molecule that specifically binds to the target site) from binding to a target site. It will be appreciated by one of skill in the art that it is not necessary that the complementarity between a candidate chemical compound and a target site extend over all residues specified here in order to inhibit or promote binding of a ligand.

In general, the design of a chemical compound possessing stereochemical complementarity can be accomplished by techniques that optimize, chemically or geometrically, the “fit” between a chemical compound and a target site. Such techniques are disclosed by, for example, Sheridan and Venkataraghavan, Acc. Chem Res., vol. 20, p. 322, 1987: Goodford, J. Med. Chem., vol. 27, p. 557, 1984; Beddell, Chem. Soc. Reviews, vol. 279, 1985; Hol, Angew. Chem., vol. 25, p. 767, 1986; and Verlinde and Hol, Structure, vol. 2, p. 577, 1994, each of which are incorporated by this reference herein in their entirety.

One embodiment of the present invention for structure based drug design comprises identifying a compound (e.g., a chemical compound) that complements the shape of a component of a MEF2-MEF2 ligand complex, including a portion of MEF2 (including, but not limited to, MEF2B), HDAC (including, but not limited to, HDAC9, MITR isoform) or Cabin1. Such method is referred to herein as a “geometric approach”. In a geometric approach, the number of internal degrees of freedom (and the corresponding local minima in the molecular conformation space) is reduced by considering only the geometric (hard-sphere) interactions of two rigid bodies, where one body (the active site) contains “pockets” or “grooves” that form binding sites for the second body (the complementing molecule, such as a ligand).

The geometric approach is described by Kuntz et al., J. Mol. Biol., vol. 161, p. 269, 1982, which is incorporated by this reference herein in its entirety. The algorithm for chemical compound design can be implemented using the software program DOCK Package, Version 1.0 (available from the Regents of the University of California). Pursuant to the Kuntz algorithm, the shape of the cavity or groove on the surface of a structure (e.g., MEF2-MEF2 ligand complex) at a binding site or interface is defined as a series of overlapping spheres of different radii. One or more extant databases of crystallographic data (e.g., the Cambridge Structural Database System maintained by University Chemical Laboratory, Cambridge University, Lensfield Road, Cambridge CB2 1EW, U.K.) or the Protein Data Bank maintained by Brookhaven National Laboratory, is then searched for chemical compounds that approximate the shape thus defined.

Chemical compounds identified by the geometric approach can be modified to satisfy criteria associated with chemical complementarity, such as hydrogen bonding, ionic interactions or Van der Waals interactions.

Another embodiment of the present invention for structure based identification of compounds comprises determining the interaction of chemical groups (“probes”) with an active site at sample positions within and around a binding site or interface, resulting in an array of energy values from which three dimensional contour surfaces at selected energy levels can be generated. This method is referred to herein as a “chemical-probe approach.” The chemical-probe approach to the design of a chemical compound of the present invention is described by, for example, Goodford, J. Med. Chem., vol. 28, p. 849, 1985, which is incorporated by this reference herein in its entirety, and is implemented using an appropriate software package, including for example, GRID (available from Molecular Discovery Ltd., Oxford OX2 9LL, U.K.). The chemical prerequisites for a site-complementing molecule can be identified at the outset, by probing the active site of a MEF2 complex or component thereof, for example, (e.g., as represented by the atomic coordinates shown in one of PDB Accession Nos. 1TQE or 1N6J) with different chemical probes, e.g., water, a methyl group, an amine nitrogen, a carboxyl oxygen and/or a hydroxyl. Preferred sites for interaction between an active site and a probe are determined. Putative complementary chemical compounds can be generated using the resulting three dimensional pattern of such sites.

According to the present invention, suitable candidate compounds to test using the method of the present invention include proteins, peptides or other organic molecules, and inorganic molecules. Suitable organic molecules include small organic molecules. Peptides refer to small molecular weight compounds yielding two or more amino acids upon hydrolysis. A polypeptide is comprised of two or more peptides. As used herein, a protein is comprised of one or more polypeptides. Preferred therapeutic compounds to design include peptides composed of “L” and/or “D” amino acids that are configured as normal or retroinverso peptides, peptidomimetic compounds, small organic molecules, or homo- or hetero-polymers thereof, in linear or branched configurations. Suitable compounds for design or identification are described in detail below.

Preferably, a compound that is identified by the method of the present invention originates from a compound having chemical and/or stereochemical complementarity with a site on one or more components of an HDAC/MEF2/DNA complex or a Cabin1/MEF2/DNA complex as described herein. Such complementarity is characteristic of a compound that matches the surface of the protein(s) either in shape or in distribution of chemical groups and binds to protein(s) to regulate (e.g., by inhibition or stimulation/enhancement) binding of a MEF2 to one or more of its cognate ligands, for example, or to otherwise inhibit the biological activity of MEF2 or one or more of its cognate ligands. More preferably, a compound that binds to a binding site on either MEF2 or its cognate ligand associates with an affinity of at least about 10⁻⁶ M, and more preferably with an affinity of at least about 10⁻⁷M, and more preferably with an affinity of at least about 10⁻⁸ M.

Preferably, the following general sites of a MEF2/MEF2-ligand/DNA complex or components thereof are targets for structure based drug design or identification of candidate compounds and lead compounds (also referred to herein as target sites or active sites), although other sites may become apparent to those of skill in the art based on the three-dimensional structures provided herein. Although many of the sites described below are illustrated with respect to the specific amino acid sequence of a particular HDAC, MEF2 or Cabin1 protein, because the tertiary structures are predicted to be highly similar in homologous target sites on other highly related proteins and complexes (e.g., the homologous protein in different mammalian species; different MEF2 proteins that are structurally related; different HDAC proteins that are structurally related, and particularly the class II HDAC proteins (e.g., any isoform of HDAC4, HDAC5, HDAC7 or HDAC9); any isoform of Cabin1), it is to be understood that the description of the target sites is intended to encompass all other such homologues of the exemplified sequences and structures. One of skill in the art can readily extrapolate the amino acid residues within a sequence described herein to the corresponding amino acid residues in a highly related sequence simply by aligning the related sequences. More specifically, one of skill in the art can readily determine whether a given sequence aligns with another sequence, as well as identify conserved regions of sequence identity or homology within sequences, by using any of a number of software programs that are publicly available. For example, one can use BLOCKS (GIBBS) and MAST (Henikoff et al., 1995, Gene, 163, 17-26; Henikoff et al., 1994, Genomics, 19, 97-107), typically using standard manufacturer defaults.

An example of the ability to align sequences and determine relevant target sites with regard to MEF2 binding is illustrated in FIG. 4. Referring to FIG. 4, a hyphen represents identity to the top sequences in the sequence alignment shown here. The MEF2-binding residues in Cabin1 (SEQ ID NO:17), HDAC4 (SEQ ID NO:18), HDAC5 (SEQ ID NO:19), and HDAC9 (SEQ ID NO:20) are shown. The amphipathic α-helix is shown as a bar above the sequence. A putative MEF2-binding motif in p300 (SEQ ID NO:21) is also listed. The sequence of HDAC4 corresponds to residues 161-186 of the HDAC sequence.

The preferred target sites include, but are not limited to: (1) at least a portion of the interface between a dimer of MEF2 proteins and HDAC or Cabin1; (2) a ligand groove formed by the H2 helices and a β-sheet comprising the S3 β-strands of a MEF2 dimer; (3) a surface groove formed by β-strands S2 and S3 and linkers between S2, H2, and S3 of a MEF2 dimer; (4) one or both MEF2S domains of a MEF2 dimer; (5) an amphipathic helix of HDAC or Cabin1 that binds to a hydrophobic ligand groove formed by the H2 helices of a MEF2 dimer; (6) the hydrophobic face of such an amphipathic helix of HDAC or Cabin1 as described in (5); (7) at least a portion of an amphipathic helix of Cabin1 comprising or aligning with amino acids represented by SEQ ID NO:17; (8) a beta sheet-like interaction between loop I of MEF2 and the N-terminal tail of HDAC; (9) the first 200-250 N-terminal amino acids of an HDAC; (10) the first 160 N-terminal amino acid residues of an HDAC; (11) at least a portion of an amphipathic helix of an HDAC comprising or aligning with amino acids represented by any one of SEQ ID NO:18, SEQ ID NO:19 or SEQ ID NO:20. These target sites are described in detail in the Examples and the Figures. Combinations of any of these general sites are also suitable target sites. These sites are generally referenced with regard to the tertiary structure of the sites. Even if some of such sites were generally known or hypothesized to be important sites prior to the present invention based on the linear sequence and mutational analysis or binding studies of MEF2, Cabin1 or any HDAC, the present invention actually defines the sites in three dimensions and confirms or newly identifies residues that are important targets that could not be confirmed or identified prior to the present invention. The use of any of these target sites as a three dimensional structure is novel and encompassed by the present invention. Many of these target sites are further described below and illustrated in the Figures and Examples of the invention.

The Examples section and the discussion above and below provide specific detail regarding the structure of the complexes of Cabin1/MEF2/DNA or HDAC/MEF2/DNA and target sites of these complexes and components thereof based on the three-dimensional structures described herein, including the identification of important residues in the structures. It is to be understood, however, that one of skill in the art, using the description of these specific structures provided herein, will be able to identify compounds that are potential candidates for modulating the biological activity of these and other related complexes and components (e.g., other class II HDACs, such as any isoform or splice variant of HDAC4, HDAC5, or HDAC7 and other isoforms of HDAC9 which have homologous residues in many of the target sites and which can easily be modeled now using the template of atomic coordinates disclosed herein for the HDAC9 isoform MITR). All such embodiments are encompassed by the present invention.

Particularly preferred MEF2 residues that could be targeted for inhibitor or other regulatory compound design include or- align with, but are not limited to (with respect to SEQ ID NO:2): Gln56, Met62, Asp63, Leu66, Leu67, Tyr69, Thr70, Tyr72, Ser73, Glu74, Pro75, and Ser78. Although these are amino acid positions with respect to SEQ ID NO:2, which is human MEF2B, it is to be understood that the corresponding target residues can now be easily determined in other MEF2 proteins, such as MEF2A, and in MEF2 proteins from other species (e.g., mouse MEF2).

Particularly preferred Cabin1 residues that could be targeted for inhibitor or other regulatory compound design include or align with, but are not limited to (with respect to SEQ ID NO:14): Lys2161, Gly2162, Ser2163, Ile2164, Thr2168, Lyd2169, Lys2171, Leu2172, Lys2173, Ile2176 Leu2177, Ser2182, Ala2182, Ala2183, and Asn2184.

Particularly preferred HDAC residues that could be targeted for inhibitor or other regulatory compound include or align with, but are not limited to: Val143, Lys144, Lys146, Leu147, Gln148, Phe150, Leu151, and Phe177. Although these are amino acid positions with respect to SEQ ID NO:4, which is murine MITR, it is to be understood that the corresponding target residues can now be easily determined in other HDAC proteins, such as HDAC4, HDAC5, HDAC7, other isoforms of HDAC9, and in HDAC proteins from other species (e.g., human HDAC). Indeed, FIG. 4 illustrates how one of skill in the art can readily identify homologous regions of the related HDAC proteins and even extend the homology to other MEF2 ligands, such as Cabin1 and p300.

A candidate compound for binding to or otherwise modulating (regulating, modifying, upregulating, downregulating) the activity of a protein or complex of the invention, including to one of the preferred target sites described above, is identified by one or more of the methods of structure-based identification discussed above. As used herein, a “candidate compound” refers to a compound (including, but not limited to, peptides, oligonucleotides, carbohydrates and/or synthetic organic molecules) that is selected by a method of structure-based identification described herein as having a potential for binding to a component of a MEF2-MEF2 ligand-DNA complex or component thereof on the basis of a predicted conformational interaction between the candidate compound and the target site used in the method of identification. The ability of the candidate compound to actually bind to the protein or target site can be determined using techniques known in the art, as discussed in some detail below. A “putative compound” is a compound with an unknown regulatory activity, at least with respect to the ability of such a compound to bind to and/or regulate MEF2 or a cognate ligand thereof as described herein. Therefore, a library of putative compounds can be screened using structure based identification methods as discussed herein, and from the putative compounds, one or more candidate compounds for binding to, otherwise regulating, or even mimicking the target protein or site thereof can be identified. Alternatively, a candidate compound for binding to or mimicking a target protein or site can be designed de novo using structure based drug design, also as discussed above.

Accordingly, in one aspect of the present invention, the method of structure based identification of compounds that potentially bind to or modulate (regulate) the interaction of MEF2 with one or more of its cognate ligands further includes steps which confirm whether or not a candidate compound has the predicted properties with respect to its effect on the actual protein(s) or complex, preferably by synthesizing the candidate compound and conducting biological, molecular or chemical assays to select those compounds that actually have the desired activity in vitro, ex vivo or in vivo. In one embodiment, the candidate compound is predicted to be an inhibitor of the binding of MEF2 to at least one of its ligands, and the method further includes producing or otherwise obtaining a candidate compound selected in the structure based method and determining whether the compound actually has the predicted effect on MEF2, its ligand, or the complex between the two, or a biological activity resulting from the natural interaction between MEF2 and its ligand. For example, one can additionally contact the candidate compound selected in the structure based identification method with a target protein or a fragment thereof (e.g., MEF2, an HDAC, Cabin1) under conditions in which the protein binds to one or more ligands or substrates in the absence of the candidate compound; and measuring the binding affinity of the protein or fragment thereof for its ligand, substrate or a fragment thereof. In this example (binding), a candidate inhibitor compound is selected as a compound that inhibits the binding of the protein to its ligand or substrate when there is a decrease in the binding affinity of the protein or fragment thereof for the ligand, substrate or fragment thereof, as compared to in the absence of the candidate inhibitor compound.

In another embodiment, the candidate compound is predicted to inhibit the biological activity of a target protein, and the method further comprises contacting the actual candidate compound selected by the structure-based identification method with the protein(s) or a targeted fragment thereof, under conditions wherein in the absence of the compound, the protein is biologically active, and measuring the ability of the candidate compound to inhibit the activity of the protein.

In another embodiment, the candidate compound, or modeled protein structure in some embodiments, is predicted to be a mimic or homologue of the natural protein and is predicted to have modified biological activity as compared to the natural protein. For example, one can model and then produce and test an HDAC homologue that has different binding affinity or avidity for MEF2B as compared to the natural HDAC, or a homologue that has increased or decreased biological activity as compared to the natural HDAC protein. Such homologues can be useful in various biological assays, as competitive inhibitors (lead compounds or actual therapeutic compounds), or even in the production of genetically engineered organisms.

In one embodiment, the conditions under which a protein or proteins according to the present invention are contacted with a candidate compound, such as by mixing, are conditions in which the protein or proteins are not stimulated (activated) and/or bound to a natural ligand (substrate) if essentially no candidate compound is present. In one aspect, a natural ligand can be added before, simultaneously with, or after contact with the candidate compound to determine the effect of the compound on the binding of the proteins in the assay or the biological activity of one or more of the proteins or components in the assay. Alternatively, this aspect can be designed simply to determine whether the candidate compound binds to the target protein or site (i.e., in the absence of any additional testing, such as by addition of ligands).

In another embodiment, the conditions under which a protein or complex according to the present invention is contacted with a candidate compound, such as by mixing, are conditions in which the protein is normally bound by a ligand or activated if essentially no candidate compound is present. Such conditions can include, for example, contact of MEF2 or its ligand with the appropriate binding partner. In this embodiment, the candidate compound can be contacted with the protein prior to the contact of the protein with the binding partner (e.g., to determine whether the candidate compound blocks or otherwise inhibits the binding of the protein to the binding partner or the biological activity of the protein), or after contact of the protein with the binding partner (e.g., to determine whether the candidate compound downregulates, or reduces the biological activity of the protein after the initial contact with the substrates).

It is noted that the assays described herein can readily be adapted to test and select stimulatory (enhancing, activating) compounds.

Some of the methods described herein involve contacting a protein (e.g., MEF2, HDAC, Cabin1, or a portion thereof) with the candidate compound being tested for a sufficient time to allow for binding to, activation or inhibition of the protein or complex by the candidate compound. The period of contact with the candidate compound being tested can be varied depending on the result being measured, and can be determined by one of skill in the art. For example, for binding assays, a shorter time of contact with the candidate compound being tested is typically suitable, than when activation is assessed. As used herein, the term “contact period” refers to the time period during which a protein is in contact with the compound being tested. It will be recognized that shorter incubation times are preferable because compounds can be more rapidly screened.

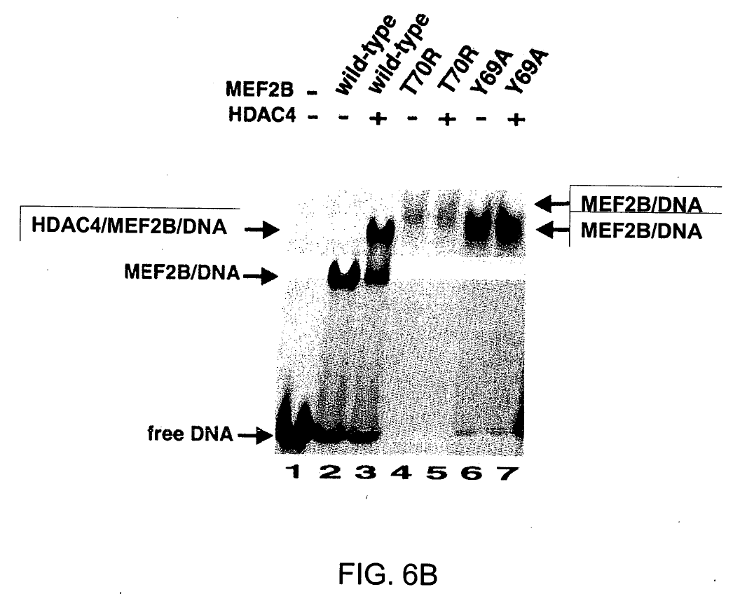

The assays of the present invention can include cell-based assays and non-cell-based assays. A particularly preferred non-cell-based assay is an electrophoresis mobility shift assay (EMSA) as described in the Examples. More specifically, the present inventors have established an electrophoresis mobility shift assay (EMSA) to monitor the formation of the ternary Cabin1/MEF2/DNA and the HDAC9/MEF2/DNA complexes. Using this technique, the inventors can form by titration both the Cabin1/MEF2/DNA (FIG. 5) and the HDAC4/MEF2/DNA complexes (not shown). By way of illustration, in FIG. 5, the electrophoresis mobility shift assay (EMSA) was performed in a buffer of 20 mM Hepes (pH 7.7), 100 mM NaCl, 1 mM DTT, and 10% glycerol. The concentration of DNA was kept at 26 μM, and approximately 52 μM of MEF2B was used in all binding reactions except the DNA control (Lane 1). The binding reactions were analyzed on a 4-20% gradient native PAGE in TBE and stained with ethidium bromide. The molar ratio of Cabin1 to the MEF2 dimer in each binding reaction is indicated on top of the gel. This method is extremely useful for screening molecular inhibitors of MEF2 that can disrupt the formation of the Cabin1/MEF2/DNA or HDAC/MEF2/DNA ternary complex but not the MEF2/DNA binary complex, for example. Several other laboratories have attempted to develop this method for inhibitor screen but have not been successful. To the present inventors' knowledge, they are the first to succeed in using EMSA to monitor the formation of the MEF2/co-regulator/DNA complex. Therefore, the inventors propose the use of this assay as a primary tool in the screening of small molecule inhibitors.

Other binding assays, and methods of determining regulation of gene expression will be apparent to those of skill in the art. In one embodiment, a BIAcore machine can be used to determine the binding constant of a complex between protein and a candidate compound or between a protein and its binding partner or ligand, for example, in the presence and absence of the candidate compound. The dissociation constant for the complex can be determined by monitoring changes in the refractive index with respect to time as buffer is passed over the chip (O'Shannessy et al. Anal. Biochem. 212:457-468 (1993); Schuster et al., Nature 365:343-347 (1993)). Contacting a candidate compound at various concentrations with the protein and monitoring the response function (e.g., the change in the refractive index with respect to time) allows the complex dissociation constant to be determined in the presence of the candidate compound.

Other suitable assays for measuring the binding of a candidate compound to a protein, and/or for measuring the ability of such compound to affect the binding of protein to its binding partner or ligand include, for example, Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry.

In one aspect, an assay for selecting a candidate compound for regulating the interaction of MEF2 with one or more of its cognate ligands can include detecting the transcription of a gene or reporter gene fused to its promoter that is known to be regulated by a complex of MEF2 and a ligand. Expression of transcripts is measured by any of a variety of known methods in the art. For RNA expression, methods include but are not limited to: extraction of cellular mRNA and Northern blotting using labeled probes that hybridize to transcripts encoding all or part of a gene; amplification of MRNA expressed from the gene using gene-specific primers, polymerase chain reaction (PCR), and reverse transcriptase-polymerase chain reaction (RT-PCR), followed by quantitative detection of the product by any of a variety of means (e.g., polyacrylamide gel analysis, chromatography or spectroscopy); extraction of total RNA from the cells, which is then labeled and used to probe cDNAs or oligonucleotides encoding all or part of the genes of this invention, arrayed on any of a variety of surfaces; in situ hybridization; and detection of a reporter gene. The term “quantifying” or “quantitating” when used in the context of quantifying transcription levels of a gene can refer to absolute or to relative quantification. Absolute quantification may be accomplished by inclusion of known concentration(s) of one or more target nucleic acids and referencing the hybridization intensity of unknowns with the known target nucleic acids (e.g. through generation of a standard curve). Alternatively, relative quantification can be accomplished by comparison of hybridization signals between two or more genes, or between two or more treatments to quantify the changes in hybridization intensity and, by implication, transcription level.

Candidate compounds identified by the present invention can include agonists of MEF2 or MEF2 ligand activity and antagonists of MEF2 or MEF2 ligand activity, with the identification of antagonists or inhibitors being preferred. As used herein, the phrase “agonist” refers to any compound that interacts with MEF2 or a MEF2 ligand and elicits an observable response. More particularly, a MEF2 or MEF2 ligand agonist can include, but is not limited to, a protein (including an antibody), a peptide, a nucleic acid or any suitable product of drug design (e.g., a-mimetic) which is characterized by its ability to agonize (e.g., stimulate, induce, increase, enhance) the biological activity of a naturally occurring MEF2 or MEF2 ligand in a manner similar to a natural agonist. An “antagonist” refers to any compound which inhibits the biological activity of MEF2 or a MEF2 ligand and particularly, which inhibits the effect of the interaction of MEF2 with its natural substrates. More particularly, a MEF2 or MEF2 ligand antagonist (e.g., an inhibitor) is capable of associating with MEF2 or MEF2 ligand such that the biological activity of the protein is decreased (e.g., reduced, inhibited, blocked, reversed, altered) in a manner that is antagonistic (e.g., against, a reversal of, contrary to) to the natural activity of the protein (e.g., the activity induced under normal conditions in the presence of natural substrates or ligands). It is noted that the three dimensional structures disclosed herein can be used to design or identify candidate compounds that agonize or antagonize the interactions of MEF2 with its cognate ligands.

The atomic coordinates that define the three dimensional structure of MEF2 and its ligands, Cabin1 and MITR in complex with DNA, and the step of obtaining such coordinates have been described in detail previously herein with regard to the method of structure based identification of compounds. Computer modeling methods suitable for modeling the atomic coordinates to identify sites in the structures described herein that are predicted to contribute to the biological activity of such complexes and similar complexes, as well as for modeling homologues of the components that are predicted to interact with their binding partner, have been discussed generally above. A variety of computer software programs for modeling and analyzing three dimensional structures of proteins are publicly available. The Examples section describes in detail the use of a few of such programs to analyze the three dimensional structures. Such computer software programs include, but are not limited to, the graphical display program O (Jones et. al., Acta Crystallography, vol. A47; p. 110, 1991), the graphical display program GRASP, MOLSCRIPT 2.0 (Avatar Software AB, Heleneborgsgatan 21C, SE-11731 Stockholm, Sweden), CNS (Brunger, et al. Crystallography & NMR system: A new software suite for macromolecular structure determination. Acta Crystallogr D Biol Crystallogr 54, 905-21. (1998)), the program CONTACTS from the CCP4 suite of programs (Bailey, 1994, Acta Cryst. D50:760-763), or the graphical display program INSIGHT.

The present inventors have identified multiple sites on MEF2 and its binding partners Cabin1 and MITR, which are believed to contribute to the biological activity of complexes of these proteins with DNA. These sites and amino acid positions have been discussed in detail above and in the Examples. Using similar methods of analysis of the models, one can identify or further analyze sites on the proteins, complexes, or on other models of related proteins and complexes (e.g., other class II HDAC proteins and their complex with MEF2 proteins) which are predicted to affect (contribute to) the interaction and/or biological activity of such proteins and complexes.

Once target sites for modification on the proteins described herein are identified, homologues having modifications at these sites can be produced and evaluated to determine the effect of such modifications on biological activity. In one embodiment, a homologue of MEF2 or one or more of its ligands (e.g., an HDAC, Cabin1, p300, etc.) can be modeled on a computer to produce a computer model of a homologue which predicts the effects of given modifications on the structure of the protein and its subsequent interaction with other molecules. Such computer modeling techniques are well known in the art.

In another aspect, or subsequent to an initial computer generation and evaluation of a homologue model, an actual homologue protein can be produced and evaluated by modifying target sites of a natural protein to produce a modified or mutant protein. Homologues of the present invention can be produced using techniques known in the art including, but not limited to, direct modifications to the protein or modifications to the gene encoding the protein using, for example, classic or recombinant DNA techniques to effect random or targeted mutagenesis. Examples of the modification of HDAC4 based on the structural information derived from the complex of HDAC9 (MITR) with MEF2B are described in the Examples.

Therefore, the present invention also includes producing a model of a three-dimensional structure of a MEF2, HDAC, Cabin1 protein, other MEF2 ligand, or complex of a MEF2 protein with the MEF2 ligand, other than those specifically exemplified in the resolved crystal structures disclosed herein. The present inventors have provided detailed information regarding the tertiary structure of MEF2B and Cabin 1 or the HDAC9 isoform MITR as they exist in conformation with one another and with DNA. This structural information can be used to model other MEF2 proteins, other HDAC proteins, and particularly other class II HDAC proteins, and even other MEF2 ligands, or portions of any of such molecules. Such models can then be used to perform structure-based identification of compounds that regulate the interaction of the modeled proteins or complexes as for the originally crystallized structure.

By way of a more particular example, the atomic coordinates of the three-dimensional structures of the Cabin1/MEF2/DNA complex and/or the HDAC/MEF2/DNA complex can be used as the template to build a three-dimensional model of other related complexes, such as a MEF2 protein in complex with a different class II HDAC. Secondary structure prediction can be performed on the additional models and the validity of the alignments of primary structures can be performed. For example, secondary structure prediction methods include, but are not limited to, PHD (B. Rost et al., CABIOS, vol. 10, 266-275(1994)) and PREDATOR (D. Frishman and P. Argos, Proteins, vol. 27, 329-335(1997)). More specifically, a program such as MODELER (A. Sali and T. L. Blundell, J. Mol. Biol., vol. 234, 779-815(1993)) implemented in the InsightII_Homology software package (Insight II (97.0), MSI, San Diego) can be used to generate three-dimensional models of other related proteins and complexes using a number of different initial sequence alignments and the structural templates of the crystallized proteins and complexes. Multiple Modeler runs can be generated, and the best model from these runs is selected. The criteria for judging the “best” model can include the lowest value of the Modeler objective function, “well-behaved” PROSAII (M. Sippl, Proteins, vol. 17, 355-362(1993)) residue energy plot for the model (for example, negative residue energy scores throughout the sequence), and “well-behaved” PROFILES-3D (J. U. Bowie et al., Science, vol. 253, 164-170(1991)) local 3D-1D compatibility score plot (for example, positive plot scores throughout the sequence). Then, a program such as Modeler can be used to generate multiple different structural models of the protein or complex using the sequence alignment and template selected above, and using the determined parameter values and options. The model with the lowest—ln(Mpdf) value is then selected as the template to generate structural models of the protein or complex sequence in the next cycle of Modeler runs. At the end of several of such cycles, the “best” three-dimensional model of the structure is selected as the final structural model of the protein or complex, and the corresponding heavy (non-hydrogen) atom cartesian coordinates are determined. The structure can be further validated with the programs PROSAII, PROFILES-3D, and PROCHECK (R. M. Laskowski et al., J. Appl. Cryst. vol. 26, 283-291(1993)). Other methods of producing a model of a related protein based on a template of structural coordinates will be known in the art.

One embodiment of the present invention relates to an isolated homologue (e.g., mutant) of MEF2, an HDAC (preferably a class II HDAC such as HDAC4, HDAC5, HDAC7 or HDAC9), Cabin1, or of another MEF2 binding partner (e.g., p300) which comprises at least one amino acid modification as compared to the naturally occurring protein, or portion of such a homologue that contains the modification. Such a homologue preferably has modified biological activity, including, but not limited to, modified biological activity, modified substrate binding, and/or modified substrate specificity, as compared to the wild-type protein, or equivalent fragment/portion of the wild-type protein. The modifications to the amino acid sequence of the homologue can include modifications within any of the target sites of target amino acid residues that have been identified herein by the determination of the three-dimensional structure of the complexes of Cabin1/MEF2/DNA or MITR/MEF2/DNA.

As described above, MEF2 has been implicated in regulating the hypertrophic response in cardiac muscles. Heart hypertrophy can lead to heart failure, myocardial infarction and many different cardiovascular diseases. For this reason, small molecule inhibitors of the MEF2 binding of co-regulators including class II HDACs and p300 are desired as a means to prevent and treat cardiomyopathies associated with MEF2 regulatory activity, as well as for treating any other conditions or disorders associated with the regulation of MEF2 and MEF2 ligand interactions, including those related to other cell types.

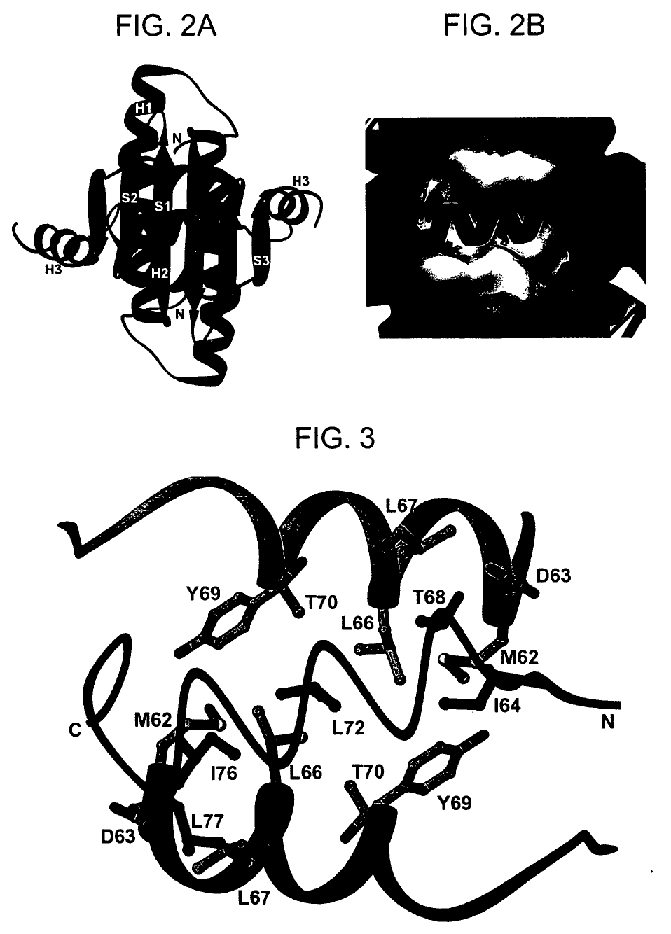

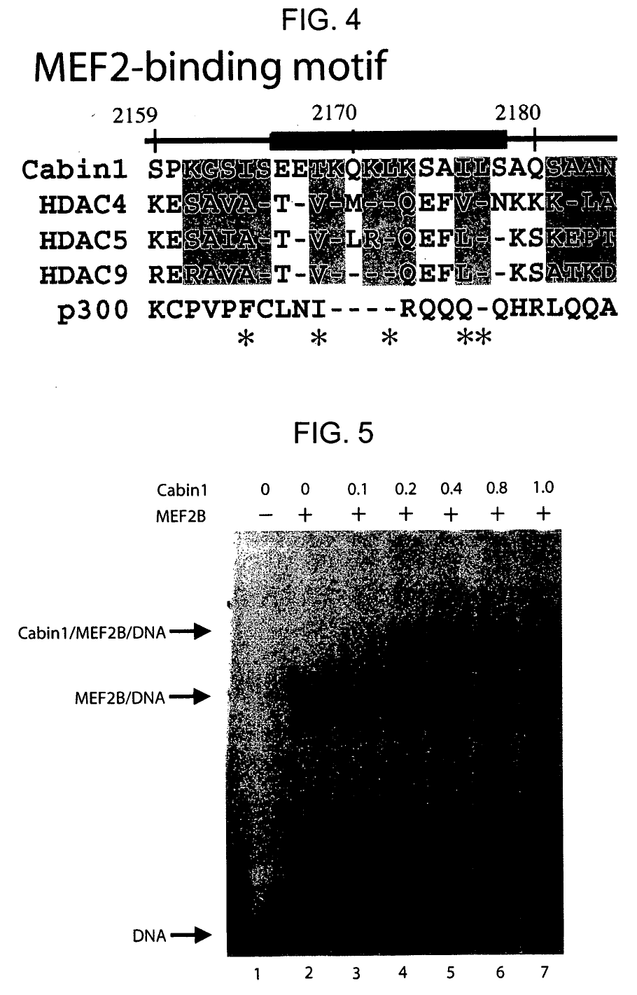

The structure of the ligand-binding pocket on MEF2 is shown in FIG. 2. A schematic of Cabin 1 in the MEF2 binding pocket is depicted in FIG. 3. As shown in FIG. 3, a lipophilic side chain on Cabin 1 interacts strongly with a hydrophobic pocket formed by the Leu66, Tyr69 and Thr70 residues on each MEF2 monomer. Similarly, Met62, Leu66 and Leu67 of one MEF2 monomer form another hydrophobic pocket with Tyr69 of the other MEF2B monomer. Lipophilic, aliphatic side chains of Cabin 1 also bind in this second hydrophobic pocket.

Structure-based strategies for designing MEF2-binding inhibitors are realized in view of the crystallographic structures of the present invention. Specifically, hydrophobic functional groups resembling the amino acid side chains of Cabin1 (Val, Leu and Ile) and aromatic functional groups similar to amino acids such as Phe and Try can be combined in small molecules that attain the correct orientation to insert into the hydrophobic pocket formed by L66, L67 and M62 on each monomer of the MEF2 binding site and a pocket formed by L66, T70 and Y69 of both MEF2 monomers. Additionally, the aromatic functional groups on these molecules are designed to interact with Y69 of each monomer respectively. These small molecules designed by molecular modeling can then be synthesized and tested in the electrophoresis mobility shift assay described above.

The features of the ligand-binding pocket disclosed above have lead to the design and synthesis of compounds depicted generally as 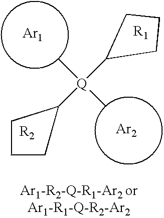
in which Ar₁ and Ar₂ are C₅-C₁₀ aromatic, C₅-C₁₀ heterocyclic or arylalkyl, R₁ and R₂ are independently C₁-C₁₀ alkyl or alkylene, and Q is C, C═C, C₁-C₁₀ alkyl or phenyl. Thus, R₁ and R₂ and Q are lipophilic groups that will insert into the hydrophobic pockets of the MEF2 binding site similar to Cabin 1 while Ar₁ and Ar₂ are aromatic groups that are positioned to interact with tyrosine residue at position 69 of each MEF2 monomer. These molecules are therefore designed to bind or occupy the protein binding site of MEF2 dimer and inhibit the interaction with Cabin 1, class II HDACs, and p300.

“Alkyl” groups according to the present invention are aliphatic hydrocarbons which can be straight or branched chain groups. Alkyl groups optionally can be substituted with one or more substituents, such as a halogen, alkenyl, alkynyl, aryl, hydroxy, amino, alkoxy, carboxy or cycloalkyl. There may be optionally inserted along the alkyl group one or more sites of unsaturation giving rise to one or more double or triple bonds. Exemplary alkyl groups include methyl, ethyl, isopropyl, n-butyl, t-butyl, pentane, 2-methylpentane, 3-methylpentane, 4-methylpentane, hexane, and heptane.

“Aryl” groups are monocyclic or bicyclic carbocyclic or heterocyclic aromatic ring moieties. Aryl groups can be substituted with one or more substituents, such as a halogen, alkenyl, alkyl, alkynyl, hydroxy, amino, thio, alkoxy or cycloalkyl.

“Heterocyclic” refers to an aromatic ring having at least one non-carbon ring substituent. Exemplary heterocyclic rings include pyrrole, thiophene, furan, imidazole, pyrazole, pyridine, pyrimidine, pyridazine, thiazole, isothiazole, oxazole, isoxazole, quinoline, isoquinoline and indole.

“Di-aryl or heteroaryl” means a bicyclic ring system composed of two fused carbocyclic and/or heterocyclic aromatic rings. Exemplary di-aryl or heteroaryl rings include indene, isoindene, benzofuran, dihydrobenzofuran, benzothiophene, indole, 1H-indazole, indoline, azulene, tetrahydroazulene, benzopyrazole, benzoxazole, benzoimidazole, benzothiazole, 1,3-benzodioxole, 1,4-benzodioxan, purine, naphthalene, tetralin, coumarin, chromone, chromene, 1,2-dihydrobenzothiopyran, tetrahydrobenzothiopyran, quinoline, isoquinoline, quinazoline, pyrido[3,4-b]-pyridine, and 1,4-benisoxazine.

“Aralkyl” refers to an alkyl group substituted with an aryl group. Suitable aralkyl groups include, without limitation, benzyl, methylene indole and phenylethyl. Aralkyl groups may also be substituted with other suitable functional groups. Aralkyl groups include those with heterocyclic and carbocyclic aromatic moieties.

The compounds of present invention may be prepared by both conventional and solid phase synthetic techniques known to those skilled in the art. Useful conventional techniques include those disclosed by U.S. Pat. Nos. 5,569,769 and 5,242,940, and PCT publication No. WO 96/37476, all of which are incorporated herein in their entirety by this reference.

Combinatorial synthetic techniques, however, are particularly useful for the synthesis of the compounds of the present invention. See, e.g., Brown, Contemporary Organic Synthesis, 1997, 216; Felder and Poppinger, Adv. Drug Res., 1997, 30, 111; Balkenhohl et al., Angew. Chem. Int. Ed. Engl., 1996, 35, 2288; Hermkens et al., Tetrahedron, 1996, 52, 4527; Henrkens et al., Tetrahedron, 1997, 53, 5643; Thompson et al., Chem. Rev., 1996, 96, 555; and Nefzi et al., Chem. Rev., 1997, 2, 449-472.

The compounds of the present invention can be synthesized from readily available starting materials. Various substituents on the compounds of the present invention can be present in the starting compounds, added to any one of the intermediates or added after formation of the final products by known methods of substitution or conversion reactions. If the substituents themselves are reactive, then the substituents can themselves be protected according to the techniques known in the art. A variety of protecting groups are known in the art, and can be employed. Examples of many of the possible groups can be found in “Protective Groups in Organic Synthesis” by T. W. Green, John Wiley and Sons, 1981, which is incorporated herein in its entirety. For example, nitro groups can be added by nitration and the nitro group can be converted to other groups, such as amino by reduction, and halogen by diazotization of the amino group and replacement of the diazo group with halogen. Acyl groups can be added by Friedel-Crafts acylation. The acyl groups can then be transformed to the corresponding alkyl groups by various methods, including the Wolff-Kishner reduction and Clemmenson reduction. Amino groups can be alkylated to form mono-and di-alkylamino groups; and mercapto and hydroxy groups can be alkylated to form corresponding ethers. Primary alcohols can be oxidized by oxidizing agents known in the art to form carboxylic acids or aldehydes, and secondary alcohols can be oxidized to form ketones. Thus, substitution or alteration reactions can be employed to provide a variety of substituents throughout the molecule of the starting material, intermediates, or the final product, including isolated products.

Since the compounds of the present invention can have certain substituents which are necessarily present, the introduction of each substituent is, of course, dependent on the specific substituents involved and the chemistry necessary for their formation. Thus, consideration of how one substituent would be affected by a chemical reaction when forming a second substituent would involve techniques familiar to one of ordinary skill in the art. This would further be dependent upon the ring involved.

It is to be understood that the scope of this invention encompasses not only the various isomers which may exist but also the various mixtures of isomers which may be formed.

If the compound of the present invention contains one or more chiral centers, the compound can be synthesized enantioselectively or a mixture of enantiomers and/or diastreomers can be prepared and separated. The resolution of the compounds of the present invention, their starting materials and/or the intermediates may be carried out by known procedures, e.g., as described in the four volume compendium Optical Resolution Procedures for Chemical Compounds: Optical Resolution Information Center, Manhattan College, Riverdale, N.Y., and in Enantiomers, Racemates and Resolutions, Jean Jacques, Andre Collet and Samuel H. Wilen; John Wiley & Sons, Inc., New York, 1981, which are incorporated herein in their entirety. Basically, the resolution of the compounds is based on the differences in the physical properties of diastereomers by attachment, either chemically or enzymatically, of an enantiomerically pure moiety results in forms that are separable by fractional crystallization, distillation or chromatography.

When the compounds of the present invention contain an olefin moiety and such olefin moiety can be either cis- or trans-configuration, the compounds can be synthesized to produce cis- or trans-olefin, selectively, as the predominant products. Alternatively, the compound containing an olefin moiety can be produced as a mixture of cis- and trans-olefins and separated using known procedures, for example, by chromatography as described in W. K. Chan, et al., J. Am. Chem. Soc., 1974, 96, 3642, which is incorporated herein in its entirety.

The compounds of the present invention form salts with acids when a basic amino function is present and salts with bases when an acid function, e.g., carboxylic acid or phosphonic acid, is present. All such salts are useful in the isolation and/or purification of the new products. Of particular value are the pharmaceutically acceptable salts with both acids and bases. Suitable acids include, for example, hydrochloric, oxalic, sulfuric, nitric, benzenesulfonic, toluenesulfonic, acetic, maleic, tartaric and the like which are pharmaceutically acceptable. Basic salts for pharmaceutical use include Na, K, Ca and Mg salts.

In addition to and/or instead of a rational drug design, other MEF2 binding inhibitors can be identified by the electrophoresis mobility shift screening process described above, in which a variety of compounds are tested to determine their MEF2 binding ability and MEF2 binding inhibition. In this manner, a variety of peptide MEF2 binding inhibitors have been identified. Thus, compounds of the present invention include substituted and unsubstituted small peptide MEF2 binding inhibitors and nucleosides and analogs thereof.

The comoupounds of the present invention can be administered to a patient to achieve a desired physiological effect. Preferably the patient is an animal, more preferably a mammal, and most preferably a human. The compound can be administered in a variety of forms adapted to the chosen route of administration, e.g., orally or parenterally. Parenteral administration in this respect includes, but is not limited to, administration by the following routes: intravenous; intramuscular; subcutaneous; intraocular; intrasynovial; transepithelially including transdermal, ophthalmic, sublingual and buccal; topically including ophthalmic, dermal, ocular, rectal and nasal inhalation via insufflation and aerosol; intraperitoneal; and rectal systemic.

The active compound can be orally administered, for example, with an inert diluent or with an assimilable edible carrier, or it can be enclosed in hard or soft shell gelatin capsules, or it can be compressed into tablets, or it can be incorporated directly with the food of the diet. For oral therapeutic administration, the active compound may be incorporated with excipient and used in the form of ingestible tablets, buccal tablets, troches, capsules, elixirs, suspensions, syrups, wafers, and the like. Such compositions and preparation can contain at least 0.1% of active compound. The percentage of the compositions and preparation can, of course, be varied and can conveniently be between about 1 to about 10% of the weight of the unit. The amount of active compound in such therapeutically useful compositions is such that a suitable dosage will be obtained. Preferred compositions or preparations according to the present invention are prepared such that an oral dosage unit contains from about 1 to about 1000 mg of active compound.

The tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active compound can be incorporated into sustained-release preparations and formulation.

The active compound can also be administered parenterally. Solutions of the active compound as a free base or pharmacologically acceptable salt can be prepared in water suitably mixed with a surfactant such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It can be stable under the conditions of manufacture and storage and must be preserved against the contaminating action of microorganisms such as bacterial and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, e.g., sugars or sodium chloride. Prolonged absorption of the injectable compositions of agents delaying absorption, e.g., aluminum monostearate and gelatin.

Sterile injectable solutions are prepared by incorporating the active compound in the required amount in the appropriate solvent with various other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile filtered solution thereof.

The therapeutic compounds of the present invention can be administered to a patient alone or in combination with pharmaceutically acceptable carriers, as noted above, the proportion of which is determined by the solubility and chemical nature of the compound, chosen route of administration and standard pharmaceutical practice.

The physician will determine the dosage of the present therapeutic agents which will be most suitable for prophylaxis or treatment and it will vary with the form of administration and the particular compound chosen, and also, it will vary with the particular patient under treatment. The physician will generally wish to initiate treatment with small dosages by small increments until the optimum effect under the circumstances is reached. The therapeutic dosage can generally be from about 0.1 to about 1000 mg/day, and preferably from about 10 to about 100 mg/day, or from about 0.1 to about 50 mg/Kg of body weight per day and preferably from about 0.1 to about 20 mg/Kg of body weight per day and can be administered in several different dosage units. Higher dosages, on the order of about 2× to about 4×, may be required for oral administration.

The following examples are provided for the purpose of illustration and are not intended to limit the scope of the application.

### EXAMPLES

### Example 1

The following example describes the crystallization, resolution of structure, and analysis of the Cabin1/MEF2/DNA complex

Methods

Sample preparation and crystallization. Human MEF2B (residues 1-93 of SEQ ID NO:2) and Cabin1 (residues 2156-2190 of SEQ ID NO:14) were cloned in pET-30b as a fusion protein with Cabin1 at the C-terminus and a PreScission site in between. The protein was expressed in E. coli BL21(DE3)pLysS and purified by ammonium sulfate precipitation and Sp sepharose. The purified fusion protein was cleaved with the PreScission Protease (Amersham Bioscience) and further purified by gel filtration. The DNA was prepared by solid phase synthesis and purified by MonoQ under denaturing conditions. The DNA sequence is shown in FIG. 1 (MEF2-binding site in bold). The Cabin1/MEF2B/DNA ternary complex was prepared by mixing 20% molar excess of DNA with the Cabin1/MEF2B complex and further purified by Prep Cell (Bio-Rad model 491) with elution buffer of 5 mM Hepes, 30 mM NaCl and 0.5 mM EDTA, and 1 mM DTT. The complex peak was then added 20% excess DNA and concentrated down to 0.4 mM for crystallization. Crystals were grown at 17° C. by the hanging drop method using a reservoir buffer of 50 mM BTP (pH 6.35-6.68), 15% PEG 1000, 50 mM NaCl, 10 mM CaCl₂, 5 mM MgCl₂, 10% Glycerol, 5 mM DTT, 2 mM Spermine. Typically, cubic-shaped crystals grew to 300 μm in two weeks. The crystals belong to the space group P4₁22, with cell dimensions a=b=70.14 Å, and c=151.88 Å.

Data collection, structure determination and analysis. Crystals were stabilized in the harvest/cryoprotectant buffer: 50 mM BTP (pH 6.68), 50 mM NaCl, 15% PEG 1K, 15% glycerol and 5 mM DTT. All crystals were flash frozen in liquid nitrogen for storage and data collection under cryogenic conditions (100 K). The data were collected at the Advanced Photon Source (APS, Argonne National Laboratory) beam line (14-BM-C). Data were reduced using the program DENZO and SCALEPACK (Otwinowski et al., 1993). The structure of the ternary Cabin1/MEF2B/DNA complex was solved by the molecular replacement method using the MEF2A/DNA binary complex as a partial search model (Santelli et al., 2000). The model building and refinement were carried out using programs O and CNS (Jones et al., 1991; Brunger et al., 1998). Throughout the refinement, NCS restraints were applied to the MEF2 dimer. At the initial stage of the molecular replacement, the “extra” electron density corresponding to the C-terminal half of the MEF2S domain and Cabin1 were clearly defined. The DNA in the crystals has two orientations; each was refined at half occupancy. The first two bases at the 5′ end and the last base at the 3′ end for both strands are disordered and not included in the refined model. Water molecules were picked using the automatic procedure implemented in the CNS program at the final stages of refinement (Brunger et al., 1998). The final model has good geometry as examined by PROCHECK (The CCP4 Suite, 1994). The R_{factor} of the refined model is 24.2%/31.3% (overall/last bin) and the R_{free} is 26.8%/34.1% (overall/last bin). The model has 3071 non-hydrogen atoms and 229 water molecules. Detailed statistics of crystallographic analysis are presented in Table 1 of Han et al., 17 Apr. 2003, Nature 422(6933):730-734, incorporated herein by reference. Figures of structure illustration were prepared using the program RIBBONS (Carson et al., 1991).

In vitro binding assay of Cabin1 mutants. The glutathione S-transferase(GST)-MEF2B fusion was expressed in E. coli DH5α and purified as previously described (Youn et al., 1999). [³⁵S]-labeled Cabin1 or its mutants was synthesized in vitro using pSG-Cabin1 (2144-2220) or the corresponding mutant plasmid (generated by Stratagene site-mutagenesis Kit) in a coupled transcription-translation system (TNT kit, Promega). Roughly equal amounts of wild type and mutant proteins were incubated with a 50% slurry of GST-MEF2B immobilized on glutathione-sepharose beads in 200 to 300 μL HEMG binding buffer (40 mM HEPES-[pH 7.8], 50 mM KCl, 5 mM MgCl₂, 0.1% Triton X-100, 10% glycerol, 1.5 mM DTT, protease inhibitors [Boehringer-Mannheim], and 0.5 mg/mL bovine serum albumin). The beads were washed four times with 1 mL (each time) HEMG buffer. Bound proteins were eluted in 30 μL 50 mM Tris-Cl (pH 7.4) containing 15 mM glutathione, resolved by SDS-PAGE and visualized by autoradiography. The control was carried out with the wild type Cabin1 (2144-2220) and beads without immobilized GST-MEF2B (data not shown).

Results and Analysis

Human MEF2B contains sequential MADS-box (residues 2-58 of SEQ ID NO:2) and MEF2-specific (MEF2S) domain (residues 59-91 of SEQ ID NO:2) that are necessary and sufficient to bind a motif in human Cabin1 (residues 2156-2190 of SEQ ID NO:14) (Youn et al., 1999) (see Han et al., 2003, Nature, supra). The inventors determined the crystal structure of their complex bound to DNA with a MEF2 site by molecular replacement using the previously determined MEF2A/DNA complex as a search model (Woronicz et al., 1995; Santelli et al., 2000). The Cabin1 fragment and the C-terminal part of the MEF2S domain that were absent in the starting model have well-defined electron density (Han et al., 2003, Nature, supra). The overall structure of the Cabin1/MEF2B complex resembles a multi-layered pyramid built on the DNA (FIG. 1). MEF2 forms a symmetric homodimer. Each MEF2 monomer consists of an extended N-terminal tail, three α-helices (H1, H2, and H3), and three β-strands (S1, S2, and S3). The N-terminal tail, helix H1 and strands S1 and S2 form the MADS-box core domain that mediates DNA recognition and dimerization in a manner similar to that observed in the MEF2A/DNA complexes (Santelli et al., 2000; Huang et al., 2000). The MEF2S domain of each monomer forms a helix-strand-helix motif (H2-S3-H3) on the surface of the MADS-box core. At the top of the pyramid sits the α-helix of Cabin1 that forms a three-helix bundle with the H2 helices (FIG. 1). Because the MADS-box and (helix-H2) of the MEF2S domain obseerved in the MEF2A/DNA complex can be superimposed very well with the corresponding part of the Cabin1/MEF2B/DNA complex (r.m.s.d 0.54 Å for 114 Cα atoms), the binding of Cabin1 apparently has little effect on the structure and function of the MADS-box of MEF2B.

A surprising structural feature of the ternary Cabin1/MEF2B/DNA complex is the entirely folded MEF2S domain, which was only partially observed (helix H2) in the MEF2A/DNA complexes with shorter MEF2 fragments (Santelli et al., 2000; Huang et al., 2000). The observed structure of the MEF2S domain in this ternary complex is most likely due to intrinsic protein folding interactions rather than crystal packing and Cabin1-binding, because the same structure is found in the two MEF2B monomers in different crystal packing environments. Moreover, Cabin1 makes no direct contact to H3 and interacts with S3 of each monomer differently, and yet the structures of S3 of both monomers are identical. The MADS-box in MCM1 and SRF frequently bind. their respective transcription partners through strand S2 and helix H1, which contain many exposed hydrophobic residues conserved between MCM1 and SRF (Tan et al., 1998; Hassler et al., 2001; Pellegrini et al., 1995). Remarkably, many of these residues, such as Met29, Tyr33, Phe55, and Tyr57 are also found in the MADS-box of MEF2. But in MEF2 the exposed hydrophobic surfaces of the MADS-box interact with the MEF2S domain of the other monomer (FIG. 1).

The detailed interactions between the MADS-box and the MEF2S domain in MEF2B, however, are strikingly similar to those used by the MADS-box of MCM1 and SRF to interact with their respective partners (Tan et al., 1998; Hassler et al., 2001). In the MEF2B dimer, strand S3 of one monomer forms parallel β-strands with S2 of the other monomer through main chain hydrogen bonds and extensive side chain interactions. Immediately following strand S3 is an amphipathic helix (H3) that docks perpendicularly on H1 of the opposing monomer through primarily hydrophobic interactions (FIG. 2c, Han et al., 2003, supra). Residues mediating the interactions between the MEF2S domain and the MADS-box are highly conserved in the MEF2 family (Han et al., 2003, supra), suggesting that members of this family can form homo- or possibly heterodimers with a similar structure.

The observed structure of the MEF2S domain has several functional implications. First, the MEF2S domain provides an interlock to further stabilize dimerization by interacting with the MADS-box of the reciprocal subunit. As a result, a much larger surface area (6228 Å²) is buried in the MEF2 dimer with an intact MEF2S domain (FIG. 1). Second, the MEF2S domain could enhance DNA binding through direct stabilization of the DNA-binding helix H1, which is clamped down by helix H3 of the MEF2S domain (Molkentin et al., 1996) (FIG. 1). Third, the MEF2S domain, which is unique to the MEF2 subfamily of the MADS-box proteins, may provide the major protein surface to interact with other transcription factors that “crosstalk” with MEF2. Indeed, mutations on MEF2C (N73I/E74A/H76L or E77V/S78N/R79Q/T80A) have been shown to disrupt its ability to activate transcription synergistically with MyoD family of transcription factors (Molkentin et al., 1995). These mutations map nicely to protein surfaces associated with strand S3 and its linker to S2 (FIG. 2d of Han et al., 2003, supra). Finally, the fully folded MEF2S domain provides a stable docking site for the transcriptional co-repressor Cabin1 and potentially other transcriptional co-regulators.

FIG. 2A and FIG. 2B are schematic drawings showing a ligand-binding pocket on MEF2. FIG. 2A shows the ligand binding groove formed by the alpha helix H2 and beta strands S1, S2 and S3 of each MEF2 monomer. The helix transversing the groove denotes the MEF2-binding motifs of Cabin1. FIG. 2B shows the hydrophobic nature (the lighter patches around the Cabin1 helix) of the MEF2 ligand-binding pocket. These figures show that Cabin1 is bound to the MEF2S domain in a deep groove with the central β-sheet as the floor and the two helices (H2) as the rim (FIG. 2A). The groove is lined with predominantly conserved hydrophobic residues that engage in extensive van der Waals interactions with Cabin1 to form a binding site that is more extended than previously predicted (Santelli et al., 2000; Huang et al., 2000) (FIG. 2B). Additional β-strands (S3) of the MEF2S domain expand the central β-sheet of the MADS-box to six strands (FIG. 2a from Han et al., 2003, supra; and FIG. 2A). A shorter MEF2B fragment (residues 2-78) missing the C-terminal half of the MEF2S domain (S3 and H3) has much weaker affinity for Cabin1 than the construct used here (supplemental FIG. 1 in Han et al., 2003, supra). Overall, the Cabin1-binding site of MEF2 resembles the peptide-binding pocket of MHC, which also has a β-sheet floor and two helix rims (Bjorkman et al., 1987). However, unlike the extended peptide ligands bound by MHC, the MEF2-binding motif of Cabin1 adopts an α-helix to form a triple helix bundle with MEF2.

The Cabin helix (residues 2166-2178)-is amphipathic. Its hydrophobic face, including residues Ile64, Thr68, Leu72, Ile76 and Leu77 (individual residues from Cabin1 are italicized throughout the text and are referred to by the last two digits of their actual sequence number), binds into the double helix cleft of MEF2 (FIG. 3). Referring to FIG. 3, residues from Cabin1 appear in the middle structure. Residues in MEF2 are labeled above and below (see also FIG. 4a from Han et al., 2003, supra, which appears in color). More particularly, located at the center of the Cabin1/MEF2 interface and approximately on the dyad axis of the MEF2 dimer is Leu72 of Cabin1, whose side chain inserts deeply into a hydrophobic pocket formed by Leu66, Tyr69, and Thr70 of each MEF2B monomer. N terminal to Leu72, Ile64 and Thr68 of Cabin1 together fill in a hydrophobic pocket formed by Met62, Leu66, and Leu67 of monomer A and Tyr69 of monomer B, whereas the dyad symmetry-related hydrophobic pocket is occupied by Ile76 and Leu77 of Cabin1. Located at the center of the Cabin1/MEF2 interface and approximately on the dyad axis of the MEF2 dimer is Leu72of Cabin1, whose side chain inserts deeply into a hydrophobic pocket formed by Leu66, Tyr69, and Thr70 of each MEF2B monomer. N terminal to Leu72, Ile64 and Thr68 of Cabin1 together fill in a hydrophobic pocket formed by Met62, Leu66, and Leu67 of monomer A and Tyr69 of monomer B, whereas the dyad symmetry-related hydrophobic pocket is occupied by Ile76 and Leu77 of Cabin1. Thus, the MEF2-binding surface of Cabin1 has a pseudo dyad symmetry that approximately matches the dyad symmetry of the MEF2 dimer, explaining the 1:2 binding ratio of Cabin1 to MEF2 observed in solution (FIG. 2 of Supplementary Information in Han et al., 2003, supra). The location of the hydrophobic pocket at the N terminus of helix H2 may account for the diagonal orientation of the Cabin1 helix (FIGS. 2A and 3). The long aliphatic side chains of polar residues surrounding Leu72, including Lys69, Lys71, and Lys73, also make extensive van der Waals contacts to MEF2, further extending the binding interface (FIGS. 1b and 4b of Han et al., 2003, supra). The peptide segments of Cabin1 flanking its amphipathic helix bind MEF2 on both sides of the MADS-box/MEF2S domain by inserting into a surface groove formed by strands S2 and S3 and the linkers between S2, H2, and S3 (FIG. 3 of Supplementary Information in Han et al., 2003, supra). Overall, the Cabin1/MEF2B interface, burying 1736 Å² solvent accessible area, is largely hydrophobic and the intimate surface complementarities are likely the major determinants of the binding specificity between Cabin1 and MEF2 (FIG. 4b in Han et al., 2003, supra).

The present inveentors' crystal structure is corroborated by mutagenesis. Mutations of key residues at the Cabin1/MEF2B interface, such as Leu72Ala, Leu72Trp, Leu72Lys or Ile76Ala, either completely abolished or significantly reduced the binding affinity of Cabin1 to MEF2B, whereas mutation of a Cabin1 residue outside the Cabin1/MEF2 interface, Gln70Ala or Gln70Trp, had little effect (FIG. 4c of Han et al., 2003, supra). Similar results were obtained when the interactions between the same set of Cabin1 mutants and MEF2B were determined by coimmunoprecipitation in whole cell lysates of Jurkat T cells (FIG. 5 of Supplementary Information in Han et al., 2003, supra). It is remarkable that the removal of a three-carbon isopropyl group from the entire interface between Cabin1 and MEF2 in the Leu72Ala mutant is sufficient to disrupt the Cabin1-MEF2 interaction, consistent with the key structural role of Leu72 in Cabin1/MEF2 interface (FIG. 3).

The residues important for Cabin1 to bind MEF2B are conserved in a region of class II HDACs that are necessary and sufficient to bind MEF2 (Sparrow et al., 1999; Miska et al., 1999; Lemercier et al., 2000; Lu et al., 2000). The Cabin1-binding residues of MEF2B are also conserved in other members of the MEF2 family (e.g., see FIG. 1A of Han et al., 2003, supra). A MEF2C triple mutant of V65A, L66S, and L67R failed to bind HDAC4 (Lu et al., 2003, supra). In our crystal structure, Leu66 and Leu67 make extensive van der Waals contacts to Ile64, Thr68, Ile76 and Leu77 of Cabin1 (FIG. 3), which are also conserved in class II HDACs (FIG. 4). Moreover, in vitro binding data suggests that Cabin1 and HDAC4 bind MEF2 in a mutually exclusive manner (data not shown). Taken together, the structural and biochemical data suggest that the triple helix bundle observed in the Cabin1/MEF2B/DNA complex is a conserved structure adaptor for MEF2 to recruit transcriptional repressor Cabin1 or class II histone deacetylases to specific promoters.

Surprisingly, the V65A/L66S/L67R triple mutation on helix H2 has been found to diminish, rather than enhance, the transactivation function of MEF2 (Molkentin et al., 1996). A plausible explanation is that the same helix in MEF2 is also involved in the recruitment of transcriptional co-activators. Indeed, the MADS-box/MEF2S domain has been shown to bind p300 competitively with Cabin1 and class II HDACs (Youn et al., 2000, J. Biol. Chem.; Youn et al., 2000, Immunity). Sequence search using the consensus MEF2-binding motif reveals a potential MEF2-binding site in a p300 fragment that binds MEF2 (Sartorelli et al., 1997) (FIG. 1a from Han et al., 2003, supra).

In summary, the present inventors' structural and biochemical studies define a new signaling domain conserved in the MEF2 family of transcription factors, whose peptide ligands may be present in a variety of signaling and transcription molecules capable of modulating MEF2-dependent transcription. There are great interests in the MEF2/class II HDAC pathway as a potential therapeutic target for heart hypertrophy (Zhang et al., 2002). Because DNA-binding and co-regulator recruitment in MEF2 are mediated by protein surfaces located on opposite sides of the MADS-box/MEF2S domain, it is possible to inhibit the recruitment of transcriptional co-regulators by MEF2 without affecting the DNA binding of all MADS-box proteins (Molkentin et al., 1996). This feature may be important for developing small molecule ligands to modulate MEF2-dependent gene expression in clinical applications.

### Example 2

The following example describes the crystallization, resolution of structure, and analysis of the MITR/MEF2/DNA complex.

Methods

Sample preparation and crystallization. Human MEF2B (residues 1-93 of SEQ ID NO:2) and MITR (residues 128-154 of SEQ ID NO:4) were cloned in pET-30b as a fusion protein with MITR at the C-terminus. The protein was expressed in Escherichia coli BL21(DE3)pLysS and purified by ammonium sulphate precipitation and Sp-Sepharose chromatography. The purified fusion protein was further purified by gel filtration. The DNA was prepared by solid-phase synthesis and purified by MonoQ under denaturing conditions. The double stranded DNA sequence, aligned, is:

| | |
|---|---|
| AAAGCTATTTATAAGCA | (SEQ ID NO:15) |
| TTCGATAAATATTCGTT | (SEQ ID NO:16) |

The MEF2B/MITR/DNA ternary complex was prepared by mixing a 20% molar excess of DNA with the MITR/MEF2B complex and was further purified by Prep Cell (Bio-Rad Model 491) with an elution buffer of 5 mM Hepes, 30 mM NaCl, 0.5 mM EDTA, 1 mM dithiothreitol (DTT). To the complex peak was then added 20% excess DNA and it was concentrated to 0.4 mM for crystallization. Crystals were grown at 17° C. by the hanging-drop method with a reservoir buffer of 50 mM Bis-Tris propane (BTP), pH 6.35-6.68, 25% PEG 1000, 50 mM NaCl, 10 mM CaCl2, 5 mM MgCl2, 5% glycerol, 5 mM DTT and 2 mM spermine. Typically, diamond crystals grew to 100-300 μm in 2 weeks. The crystals belong to space group P1, with cell dimensions: a=44.765 Å, b=66.859 Å, c=66.924 Å. alpha=76.656, beta=71.846, gamma=71.799.

Data collection, structure determination and analysis. Crystals were directly harvested from the drop without additional stabilization. All crystals were flash-frozen in liquid nitrogen for storage and data collection under cryogenic conditions (100 K). The data were collected at an X-ray source. Data were reduced with the programs DENZO and SCALEPACK (Otwinowski et al., 1993). The structure of the ternary MEF2B/MITR/DNA complex was solved by the molecular replacement method by using the MEF2A/DNA binary complex as a partial search model (Santelli et al., 2000). Model building and refinement were performed with programs O and CNS (Jones et al., 1991; Brunger et al., 1998). Throughout the refinement, non-crystallographic symmetry (NCS) restraints were applied to the MEF2 dimer. At the initial stage of the molecular replacement, the ‘extra’ electron density corresponding to the C-terminal MEF2 domain and MITR were clearly defined. The DNA in the crystals has two orientations; each was refined at half occupancy. The first two bases at the 5′ end and the last base at the 3′ end for both strands are disordered and are not included in the refined model. Water molecules were picked by using the automatic procedure implemented in CNS at the final stages of refinement (Brunger et al., 1998). The final model has good geometry, as examined by PROCHECK (The CCP4 Suite, 1994).

Structure Determination

The inventors expressed human MEF2B (residues 1-93 of SEQ ID NO:2) and mouse MITR (residues 130-158 of SEQ ID NO:4) as fusion protein. Two different linkers were designed in the complex. Because the Cabin1/MEF2/DNA complex previously provided a high quality crystal, the inventors initially used the chimeric fusion protein by replacing the Cabin1 helical with the equivalent region of MITR. The crystal diffracted to 2 Å at one direction. To validate what was observed in this type of a complex, the inventors fused the MITR right to MEF2. After micro-seeding, this type of crystal can only diffract to 3 Å at one direction. These structures were solved by molecular replacement with MEF2A/DNA as a model. The inventors therefore report here the structural information from these two complexes. Since overall structures are very similar, the better model is used for the main discussion and the other is used for cross-validation.

Overall Structure

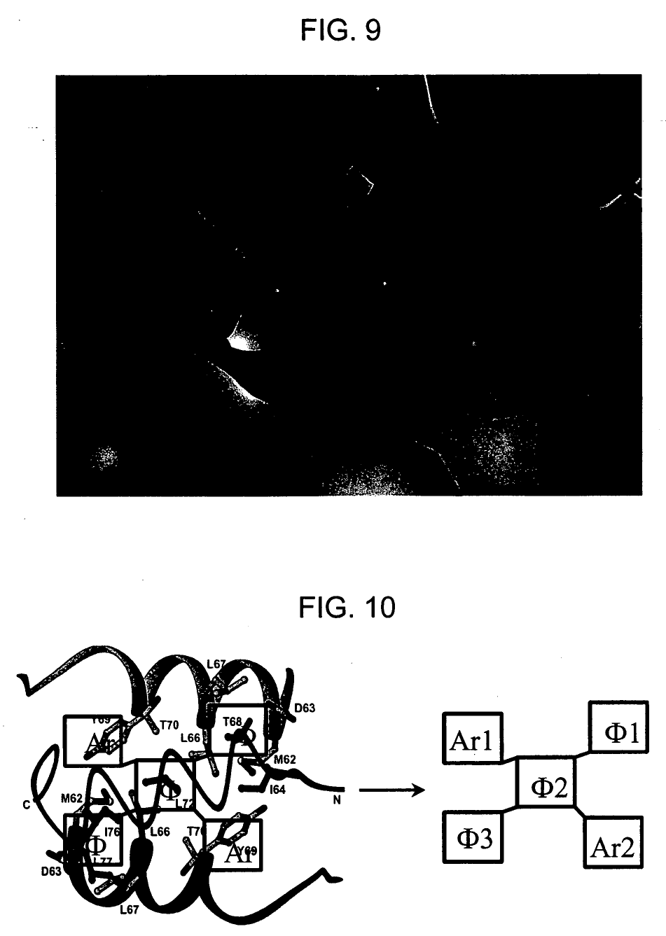

There are two tetramers in one symmetric unit. These two are nearly identical. Moreover, the MEF2 domains in two complexes have very similar conformation. The overall structure of the MEF2B/MITR/DNA complex is mainly an assembled MEF2B homodimer sitting on DNA (FIG. 8), similar to MEF2B/Cabin1/DNA. Each of MEF2B monomer consists of an extended N terminal tail, three alpha helices (H1, H2, and H3), and three beta strands (S1, S2, and S3). The tail and helix H1 provide the interface for DNA binding. Helix H1 is also part of dimer interface together with beta strands of S1 and S2. One of outstanding features of this complex is extended beta strands as compared to the original model of MEF2A/DNA. Underneath the beta sheet is a coiled coil formed by helix H1 that is oriented in parallel to the beta strands. Lying above is MEF2B helix H2 which forms a ligand binding groove in the MEF2B/Cabin1/DNA complex. Sitting at the top of the pyramid is the amphipathic helix of MITR.

MEF2A and DNA interaction has been previously very well documented (Santelli et al., 2000; Huang et al., 2000). The MEF2A/DNA complex can be superimposed well with the corresponding part of the MEF2B/MITR/DNA complex (r.m.s.d 0.004 Å). In the present inventors' ternary complex, MEF2B binds DNA primarily through the N-terminal tail inserted deeply into the minor groove and helix H1 that straddles the phosphate backbone. Residues, including Gly2, Arg3, Argl5, Lys23, Arg24, Lys30, Lys31 and Glu34, make extensive contacts with DNA in the major and minor grooves and phosphate backbone to specify the MEF2 binding sequence. The MADS-box also forms a major part of the dimer interface that further enhances MEF2/DNA binding affinity. The detailed interactions of DNA binding and dimerization by the MADS-box of MEF2B are nearly identical to that in MEF2A and also similar to that of SRF (Tan et al., 1998). Together with structure of Cabin1/MEF2/DNA, the inventors' observations suggest that the binding of ligands has little effect on the structure of the MADS-box.

MITR Ligand

The present inventors' structure of co-repressor MITR revealed detailed interactions between MEF2 and MITR. The C-terminal tail (155-158) is un-structured and not seen. The N-terminal tail in both complexes shows beta-sheet like coil.

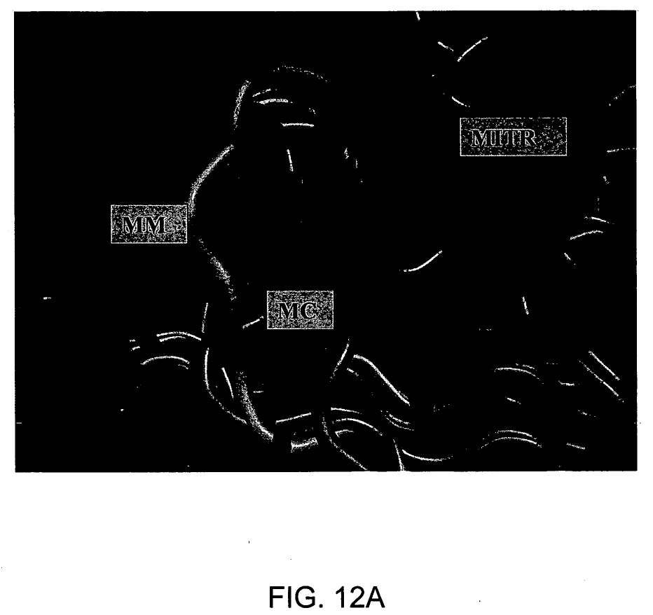

The key structure mediating MEF2/MITR interactions is the triple helix bundle formed between the amphipathic helix of MITR and two top MEF2 helices (FIGS. 11A and 11B). The hydrophobic face of the MITR helix, including Val143, Leu147, Phe150 and Leu151 fit into this groove of MEF2. Located at the center of the MEF2/MITR interface and approximately on the dyad axis of the MEF2 dimer is Leu147, whose side chain inserts deeply into a hydrophobic pocket formed by Leu66, Tyr69, and Thr70 of each monomer. The long aliphatic side chain of polar residues surrounding Leu147, including Lys144, Lys146, and Gln148 also make extensive van der Waals contacts to MEF2. Thus, similar to that in Cabin1/MEF2, the interface is mostly hydrophobic and the intimate surface complementarity contributes significantly to the binding specificity between MITR and MEF2.

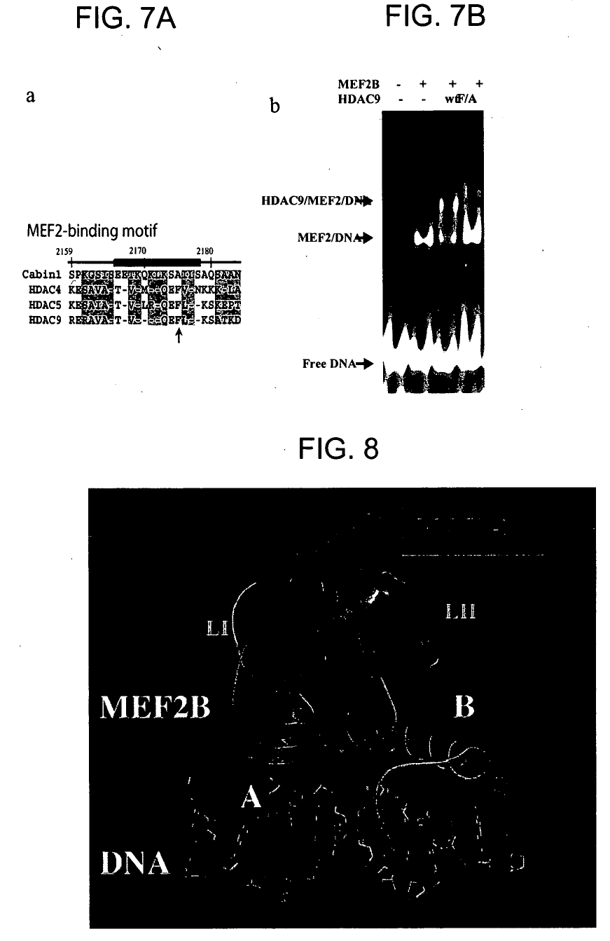

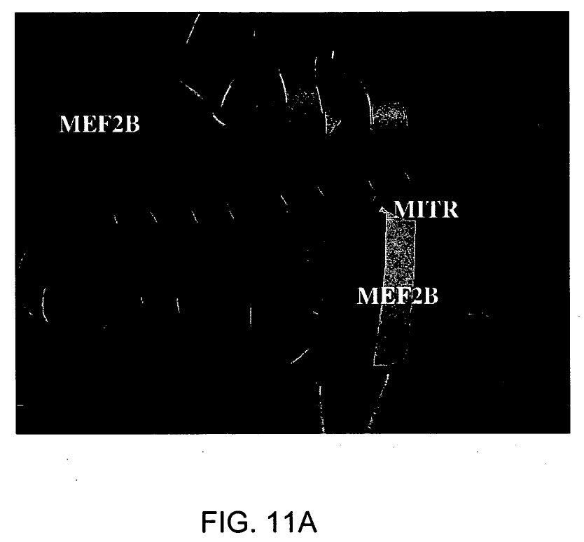

Most strikingly, the inventors observed that the Phe150 of MITR perfectly fits the hydrophobic groove of MEF2 (FIG. 9). Phe150 is also conserved in HDAC4/5/9. This is not seen in Cabin1, although overall interactions of Cabin1 are similar. The phenylalanine bears much more hydrophobicity than others, and it could thus enhance the binding of MITR. To test it, the inventors mutated this Phe150 to Ala (FIG. 7A and FIG. 7B). The binding affinity of this mutant dropped by 10 fold, suggesting Phe150 of MITR is also a very important residue in addition to Leu147 and Leu151.

To avoid the artificial observations of the MITR and MEF2 interaction, the inventors solved the crystal structure with corresponding sequence of MITR as a linker. The feature of MITR does not appear to cause any difference in their simulated omit maps, suggesting that the incorporated linkers do not affect the triple helix binding mode of MITR at all.

Plasticity of MEF2

By comparing the MEF2 bound with MITR and Cabin1, the inventors found significant changes in the local MEF2 monomer conformation between the two structures. On the one side where the N-terminus of MITR is a beta-sheet, the MEF2 loop I expanded its hydrophobic groove. The Ser73 and Pro74 move away by 4.3 A. The rearrangement makes a beta sheet-like interaction between this loop and the N-terminal tail of MITR. The main chain of the N-terminal tail of MITR not only covers the hydrophobic groove of MEF2 but also generates the three hydrogen bonds between the His76 and Thr138, Glu77 and Ser137. Consistently, the same type of interaction is also recapitulated in MITR/MEF2/DNA complex with MITR linker sequence. Moreover, Val138 fits nicely into the MEF2 hydrophobic groove while the corresponding glycine residue in Cabin1 linker simply leaves the groove open. Although this tail does not show up in one of symmetric mates, the inventors believe it is likely valid for the following reasons: 1) The MEF2 has a similar conformation even in the scenario in which the MITR tail density is missing; the tail of MITR may indeed push the loop I out; 2) This HDAC tail is apparently exposed in different crystal packing environments as compared to others. Its occupancy could thus be lower than in another environment; 3) The data the inventors have at 3.3 do not define the position with relatively low occupancy. However, further resolution will measure how much this tail contributes MITR binding.

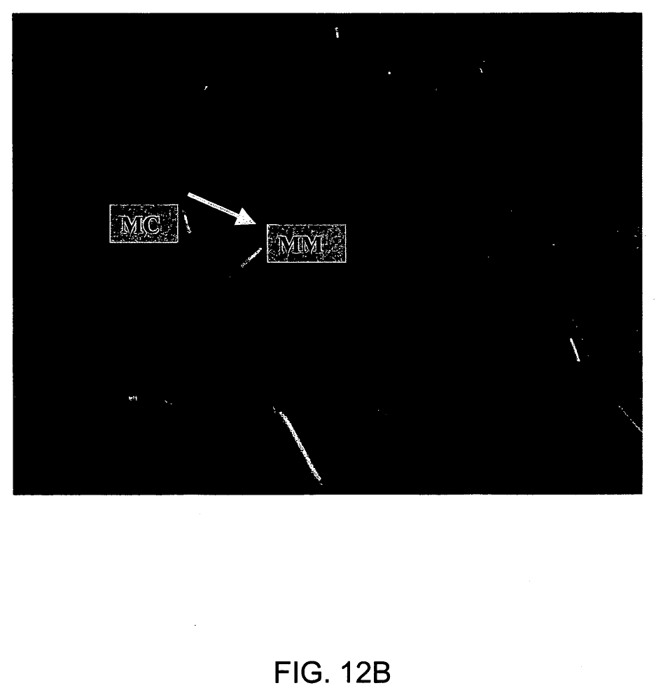

In contrast, the unstructured side of the C-terminus showed the shrunken hydrophobic groove of MEF2 (FIGS. 12A and 12B). Ser73 of MEF2 moves inward by about 3.3 A, which generates a more packed hydrophobic network of Phe150 of MITR, Tyr69 and Ser73 of MEF2. As discussed above, the Phe150 of MITR may not only make itself fit the MEF2 groove but also causes a favorable conformation change of MEF2 loop II, which further enhances MITR binding.

Additional Comparison of the HDAC9/MEF2/DNA Complex and the Cabin1/MEF2/DNA Complex

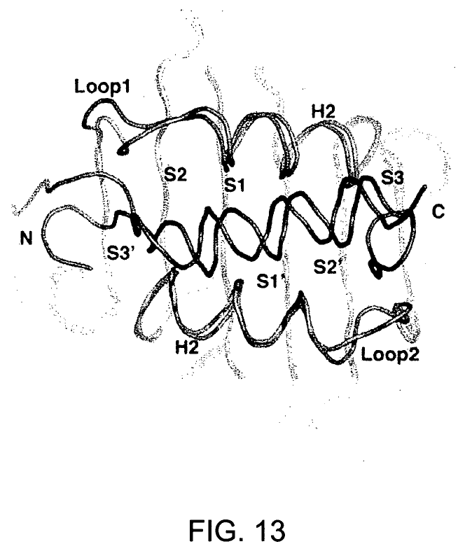

When superimposed (FIG. 13), the structures of the HDAC9/MEF2/DNA complex and the Cabin1/MEF2/DNA complex by the C alpha backbone of MEF2B (1-93). As shown in FIG. 13, the overall structures of MEF2B in the two complexes are very similar (the R.M.S.D for 186 C alpha atoms is ˜0.4 Å). However, local structures near the repressor-binding site, especially the linker between helix H2 and strand S3, referred to as the H2-S3 loop hereafter (shown as Loop1 and Loop2 for each monomer in FIG. 13), show notable differences. In the crystal structure of the Cabin1/MEF2/DNA complex, the H2-S3 loop of each monomer interacts with the N— and C-terminal tails of the amphipathic helix of Cabin1, respectively (Example 1). Because of the different sequences in these regions, the H2-S3 loop of each MEF2 monomer interacts differently with Cabin1. In the crystal structure of the HDAC9/MEF2/DNA complex, the H2-S3 loop of each MEF2 monomer even adopts distinct conformations and interacts with the N— and C-terminal tails of the HDAC9 helix differently. The conformational variations displayed by the H2-S3 loop do not appear to be crystallization artifacts because the two independent HDAC9/MEF2/DNA complexes in the asymmetric unit have almost the same variations in the H2-S3 loop region. Thus, the H2-S3 loop of MEF2 seems to play an active role in binding different sequences flanking the amphipathic helix in various MEF2-binding motifs.

The amphipathic helix f HDAC9 birids MEF2 in a manner similar to that of Cabin1 (FIG. 13). The long axis of both helices aligns diagonally between helix H2 of each MEF2 monomer. The two helices show no significant rotational displacement such that their hydrophobic faces match well with the hydrophobic groove of MEF2 similarly. However, compared with the Cabin1 helix in the Cabin1/MEF2/DNA complex, the HDAC9 helix is translated along the helical axis toward the N-terminus by about 1.5 Å. This translation is likely caused by the different MEF2-binding interactions between HDAC9 and Cabin1 (see below).

Despite the translational shift, conserved hydrophobic resides on Cabin1, such as Thr2168, Leu2172, and Ile2176, and HDAC9, such as Val143, Leu147, and Leu151, bind similar hydrophobic pockets on MEF2. However, significant differences in MEF2 binding by HDAC9 and Cabin1 are observed at non-conserved positions of the amphipathic helix. At the C-terminal end, as discussed above, a phenylalanine is conserved in HDAC4, HDAC5 and HDAC9 but not in Cabin1. In the crystal structure of the HDAC9/MEF2/DNA complex, this phenylalanine (Phe150 in HDAC9) inserts into a hydrophobic pocket (FIG. 9). In the Cabin1/MEF2/DNA complex, the corresponding residue is an alanine (Ala2175), whose side chain is too small to fill in the hydrophobic groove (data not shown). As a result, the C terminal tail of the amphipathic helix of Cabin1 folds back and partially fills in the hydrophobic pocket (FIG. 13). Interestingly, near the N-terminal end of the amphipathic helix of Cabin1, Ile2164 (see FIG. 4) inserts into a hydrophobic pocket of MEF2. In all members of class II HDACs, the corresponding residue is an alanine (FIG. 4). However, the energetic contribution of Ile2164 of Cabin1 to MEF2 binding has not been addressed. Nevertheless, comparison of the Cabin1/MEF/DNA complex and the HDAC9/MEF2/DNA complex reveal similar as well as distinct protein-protein interactions at the HDAC9/MEF2 and the Cabin1/MEF2 binding interfaces.

Discussion

The observed MITR/MEF2 interactions indicate that MITR and Class II HDACs bind to the same site on MEF2 using a conserved motif. Sequence alignment of the MEF2-binding motif in MITR and various members of the Class II HDACs showed the highest sequence similarity in the middle of the amphipathic helix, with an absolutely conserved Leu147 in the middle, and V143/F150/L151 on both sides (FIG. 4). All of these residues have homologous counterparts in class II HDACs and Cabin1. Notably, A139 of MITR points to hydrophobic groove where I106 of Cabin1 is located. Because of a lack of a side chain, the interaction of MITR tail and MEF2 is mainly mediated by the main chain, which provides a partial interpretation as to why the MITR tail has a different trajectory from that of Cabin1. Within the Class II HDACs, this tail consists of mainly small amino acids, suggesting that all class II HDACs will have a similar interaction with MEF2.

In contrast, the C-terminal flanking sequence of MITR does not appear to be involved in binding. This part of class II HDACs are less conserved than the MEF2-binding helix. Compared to Cabin1, where the Cabin1 terminal tails cover both sides of the MEF2 hydrophobic groove, MITR just leaves this groove open. This may also be the consequence of the Phe150 of MITR in place of Ala2175 of Cabin1. Cabin1 uses its tail to fit into this MEF2 groove to compensate for the lack of a Phe at this position.

Comparison of the crystal structure of the HDAC9/MEF2/DNA complex and the Cabin1/MEF2/DNA complex suggests a general model of ligand binding to the hydrophobic groove of MEF2. The concave surface of MEF2 can be viewed as an ensemble of five discrete hydrophobic pockets (labeled as A-E in FIG. 14), three of which (A, B and C) align diagonally between helix H2 of each MEF2 monomer. Their positions and shapes match well with hydrophobic residues of an amphipathic helix with the following sequence: X(V/T)KXZ(L)ZXX(V/I/L)XX (SEQ ID NO:22). The bracketed single or degenerate amino acids denote the hydrophobic residues inserting into the three hydrophobic pockets on MEF2 and K is a conserved lysine (see below), while X denotes non-conserved amino acids that may or may not interact with MEF2. The two positions sandwiching the central leucine (denoted by letter Z) are often occupied by amino acids with long aliphatic side chains, such as lysine, arginine, or glutamine (FIG. 4). These residues make extensive van der Waals contacts to Thr70and Leu67 of helix H2 of both MEF2 monomers. Mutations in these positions in Cabin1 and HDAC7 have been shown to disrupt the binding of MEF2 (Han et al., 2005, referencing Dequiedt et al., and Pan, F., Ren, R., and Liu, J. O., unpublished results). The other two pockets, labeled as D and E in FIG. 14, are also available for protein-protein interactions. One of these pockets, D, is occupied by the aliphatic side chain of a conserved lysine residue in both the HDAC9/MEF2/DNA complex and the Cabin1/MEF2/DNA complex, while the other (E) interacts with different residues in Cabin1(alanine) and HDAC9 (phenylalanine). It is possible that pockets D and E might be occupied by other hydrophobic residues or hydrophilic residues with long aliphatic side chain in MEF2-binding motifs yet to be identified. Note that the MEF2 groove has a dyad symmetry. The helix can bind MEF2 in two possible orientations by switching positions between A and C and D and E. The orientation of a co-regulator bound to MEF2 may be specified by other interactions in a fully assembled promoter complex.

It is noteworthy that a highly conserved Leucine in the MEF2 binding motif (FIG. 4, Leu180 in HDAC4) does not seem to make significant energetic contribution to MEF2 binding (FIG. 6A), despite its interaction with Leu67 of MEF2 in both the Cabin1/MEF2/DNA and the HDAC9/MEF2/DNA complexes. The reason for its conservation is not clear. Considering the broad physiological functions of MEF2 and its ability to interact with a wide range of proteins, this general model of ligand binding may help analyze the recruiting mechanism of a variety of MEF2 co-regulators. Based on the consensus MEF2-binding mode derived from our studies, the inventors have identified potential MEF2-binding sites in regions of p300 and NFAT that had been previously shown to bind the MADS-box/MEF2S domain of MEF2 (Youn et al., Sartorelli et al.). Preliminary mutational analyses indicate that these motifs in p300 and NFAT are important for MEF2 binding in vitro (Han et al. 2005, referencing others).

Comparison of the HDAC9/MEF2/DNA complex and the Cabin1/MEF2/DNA complex also reveals mechanisms of differential binding to MEF2 by distinct co-repressors. On MEF2, significant structural adaptations occur at the H2-S3 loop of MEF2, which interacts with the N— and C-terminal tails of the HDAC9 and Cabin1 helix, respectively. MEF2-binding motifs from different MEF2 co-repressors show high divergence in flanking sequences of the amphipathic helix. The observation that the H2-S3 loop of MEF2 binds the N— and C-terminal tails of the Cabin1 and HDAC9 helix in distinct conformations suggests that MEF2 may interact with these regions of various MEF2-binding motifs through an “induced-fit” mechanism. On MEF2 co-repressors, in addition to the highly conserved hydrophobic residues that bind MEF2 in a conserved manner, other less well-conserved hydrophobic residues appear to be able to “explore” the hydrophobic surface of MEF2 for additional binding sites. This mechanism of differential binding is best illustrated by the comparison of the HDAC9/MEF2 and the Cabin1/MEF2 interface. Despite the different contacts, the HDAC9/MEF2 and the Cabin1/MEF2 binding interfaces bury similar amount of surface areas (˜1476 Å² and 1736 Å², respectively), suggesting that their overall binding energies to MEF2 are similar. Interestingly, the phenylalanine conserved in HDAC4, HDAC5 and HDAC9 corresponds to a valine in HDAC7 which may not interact with the hydrophobic pocket as well as the phenylalanine. However, the MEF2-binding motif of HDAC7 contains an extra valine (indicated by a box in FIG. 1a) not found in HDAC4, HDAC5 and HDAC9. This valine is located near the N-terminus of the predicted MEF2-binding helix and may interact with MEF2 to provide additional binding energy.

Taken together, the structure and biochemical data suggest that the observed MITR/MEF2/DNA complex represents a general mechanism for sequence-specific recruitment of both-Cabin1 and class II histone deacetylases. The functions of DNA binding and co-regulator recruitment in MEF2 are well-separated in the three-dimensional structure of the ternary MITR/MEF2/DNA complex where DNA and the co-regulator are bound simultaneously on opposite protein surfaces. Thus, it is possible to inhibit the recruitment of transcription co-regulators by MEF2 without affecting the DNA binding of all MADS-box proteins.

An appropriate inhibition of HDAC activity has been of extensive interest pharmaceutically, in particular in cancer therapy and treatment for heart hypertrophy and heart failure. Even though potent candidates of small molecules to inhibit catalytic activity HDACs have shown positive effect on stimulating normal cell growth and cancer cell death, it is not yet known whether these inhibitors could benefit patients with heart hypertrophy and heart failure. Moreover, their broad association to all HDACs has caused a complicated outcome in patients treated with such compounds. On the contrary, the peptide inhibitors of MITR proposed by the present inventors can be designed or identified to just eliminate the HDAC binding to MEF2 and therefore, they are useful to establish a validated model system for high throughput screening, in addition to having a potential of becoming clinically useful drugs.

### Example 3

The following example describes mutational analyses of conserved MEF2-binding residues in HDAC4.

Methods

Electrophoresis Mobility Shift Assay

MEF2B (1-93) was expressed and purified similarly to the MEF2B/HDAC9 fusion protein. HDAC4 (155-220) Was cloned into pET28a, expressed in Escherichia coli BL21(DE3) pLysS and purified by nickel chelated agarose (Pharmacia). All HDAC4 mutants were generated by Quickchange mutation kit (Stratagene) and purified similarly to the wild type proteins. The purified wild type HDAC4 (155-220) and mutants were checked by SDS gel (bottom panel in FIG. 6A). EMSA was performed in a buffer of 20 mM Hepes (pH 7.7), 300 mM NaCl, 1 mM DTT, and 10% glycerol. The concentration of DNA was kept at 20 μM, approximately 40 μM of MEF2B was used in all binding reactions except the DNA control (Lane 1 in FIG. 6A). Approximately 20 μM of wild type HDAC4 (155-220) and its various mutants were used in the HDAC9/MEF2/DNA complex reactions (Lanes 3-9 in FIG. 6A). The binding reactions were analyzed on a 4-20% gradient native PAGE in TBE and stained with ethidium bromide.

Isothermal Titration Calorimetry (ITC)

MEF2B (1-93), HDAC4 (155-220) and its Phe178Ala mutant were prepared as described above. Before ITC analysis, all samples were dialyzed extensively against a buffer of 10 mM HEPES 7.6, 250 mM NaCl, 0.5 mM β-mercaptoethanol, 1 mM EDTA. Proteins and peptide were quantified by UV absorption at 280 nm or mass. The calorimetric titrations were carried out in a VP-ITC calorimeter (MicroCal, Northampton, Mass.) at 30° C., and the data were analyzed with the program Microcal Origin 5.0. For all experiments, MEF2B (1-93) was added to the sample cell (1.45 ml) at a concentration of 50 μM, and a 100-1500 μM solution of peptide or HDAC4 (155-220) was loaded into the 293 μl injection syringe as the titrant. While the sample cell was stirred at 300 rpm, the HDAC9 peptide or HDAC4 (155-220) was added to MEF2B (1-93) over the course of numerous injections (8-10 μl per injection) to the point that MEF2B was fully saturated. As a measure of the buffer heat of dilution, additional injections were made after the saturation was reached. In the case of the Phe178Ala mutant, because of the high concentration of the mutant protein required, the heat of dilution was significant as compared with the binding signal. Therefore, a series of controls injections were made. The heat signal of control injections were then subsequently subtracted from the raw data for final analyses. ITC analysis of the binding of HDAC4 (155-220) to MEF2B (1-93) yielded a Kd of 0.47 μM. But the measured binding stoichiometry (N˜0.2) was significantly different from the expected value (N=0.5) based on the crystal structure. The difficulty in quantifying bacterially prepared HDAC4 (155-220) might cause relatively large errors in ITC analysis.

Mutational Analyses of Conserved MEF2-binding Residues in HDAC4

Structure-guided mutational analyses of HDAC4 was performed. Since the binding of co-repressors to MEF2 does not seem to affect its DNA binding, the inventors employed an electrophoresis mobility shift assay (EMSA) to monitor the formation of the ternary HDAC4/MEF2/DNA. Using a HDAC4 (155-220) fragment encompassing the MEF2-binding motif, MEF2B (1-93) and a DNA fragment containing a consensus MEF2 site, the inventors were able to “super shift” the preformed MEF2B (1-93)/DNA complex by the HDAC4 fragment (FIG. 6A). The titration result indicates that the HDAC4/MEF2/DNA complex has a stoichiometry of 1:2:1 assuming fully active proteins and DNA (not shown), which is the same as the Cabin1/MEF2/DNA complex observed in the crystal structure (Example 1).

A series of mutants of the HDAC4 (155-220) fragment was then analyzed, including Val171Ala, Val171Lys, Leu175Ala, Leu175Lys, Val179Ala and Leu180Ala and Leu180Lys. These mutations were selected based on homologous residues in Cabin1 that are important for MEF2 binding (FIG. 4). As shown in FIG. 6A, most of the mutations disrupted the binding of HDAC4 (155-220) to the MEF2B (1-93)/DNA complex in the EMSA assay, confirming the critical roles of Leul75 and Val179. Although the Val171Ala mutation showed little effect on MEF2 binding (lane 4, FIG. 6A), the alternative mutation, Val171Lys, diminished the binding of MEF2 (lane 5, FIG. 6A), presumably due to the steric hindrance introduced by the mutation. This result suggests that Val171 is at or near the HDAC4/MEF2 binding interface. Surprisingly, despite the high conservation of Leu180 (FIG. 6A), its mutation to Ala and Lys showed small or no apparent effect on MEF2 binding (lanes 9 and 10, respectively, FIG. 6A).

The inventors also made two specific mutants of MEF2B, Thr70Arg (T70R) and Tyr69Ala (Y69A). Both Tyr69 and Thr70 make extensive contacts with Cabin1 in the Cabin1/MEF2/DNA crystal structure (Example 1). As shown in FIG. 6B, these mutations (T70R and Y69A) disrupted the binding of HDAC4 (155-220). Interestingly, the DNA complexes of both mutants showed distinct mobility from each other and from that of the wild type MEF2 (compare lanes 2, 4 and 6 in FIG. 6B). Although the possibility that the Thr70Arg or the Tyr69Ala mutation may have affected the stability of MEF2 could not formally be ruled out, the fact that both mutants retained the ability to bind DNA suggest that they are well folded. Taken together, the mutational analyses support the evidence herein that RDAC4 and Cabin1 share similar MEF2-binding-mechanisms.

Differential Binding to MEF2 by Class II HDACs and Cabin1

While the above biochemical data suggest that several key residues in class II HDACs may contact MEF2 similarly to their counterparts in Cabin1, sequence comparison reveals significant variations between Cabin1and class II HDACs in the MEF2-binding motif (FIG. 4). A phenylalanine residue conserved in HDAC4, HDAC5 and HDAC9 corresponds to an alanine in Cabin1. To test if this phenylalanine is important for MEF2 binding, the inventors made a Phe178Ala mutant of HDAC4 (155-220). The Phe178Ala mutant appeared well folded, as judged by its similar behavior on gel filtration compared to the wild type protein (data not shown). However, the binding of this HDAC4 mutant to MEF2 was significantly reduced when analyzed by EMSA (FIG. 6C). The inventors also used isothermal titration calorimetry (ITC) to analyze the binding of the wild type HDAC4 (155-220) and the Phe178Ala mutant to MEF2B (1-93). Under these experimental conditions (see above), the Kd of the wild type HDAC4 (155-220) for MEF2B (1-93) was determined to be 0.47 μM (data not shown). Titration of the Phe178Ala mutant to MEF2B (1-93) generated much weaker signal than the wild type protein. The inventors thus had to use much higher concentration of the Phe178Ala mutant (1.5 mM) than the wild type protein (0.1 mM) in ITC analyses. As a result, the heat of dilution was much more significant for the mutant protein. After correction of the heat of dilution, the Kd of the Phe178Ala mutant for MEF2B (1-93) was estimated to be ˜5 μM (data not shown). As an additional control, the inventors also performed ITC analysis of the Leu180Lys mutant of HDAC4 (155-220) (data not shown). Consistent with the result of EMSA (FIG. 6A), the Leu180Lys mutant (Kd˜0.81 μM) showed similar affinity for MEF2 to the wild type HDAC4 (155-220) (Kd˜0.47 μM). Notably, analyses of the ITC data showed significant error in stoichiometry (N˜0.23), probably due to impurity in HDAC4 (155-220). Thus, the measured Kd should only be considered as an approximation. However, because the errors appeared systematic and similar in the measurement of the wild type and mutant proteins, the binding constants can be compared. Thus, the Phe178Ala mutation reduces the binding of MEF2 by about 10 fold. By ITC analysis, the inventors have also found that the binding constants of HDAC4 (155-220) for free MEF2B (1-93) and the preformed MEF2B (1-93)/DNA complexes are similar (data not shown), suggesting that the binding of HDAC4 to MEF2 is independent of the binding of DNA to MEF2.

Taken together, these data indicate that HDAC4:binds MEF2 through a mechanism similar to but distinct from that of Cabin1.

### Example 4

The following example demonstrates that a 19-mer peptide of HDAC9 binds to MEF2.

Methods

Isothermal Titration Calorimetry (ITC)

The HDAC9 peptide, RAVASTEVKQKLQEFLLSK (SEQ ID NO:22), was synthesized and purified by HPLC. The ITC method is described in detail in Example 3 above. The binding of the highly purified HDAC9 peptide to MEF2B (1-93) was accurately characterized by ITC analyses.

Detailed HDAC9/MEF2 Interactions

As discussed in Example 2, the MEF2-binding motif of HDAC9 binds MEF2 as an amphipathic helix. The hydrophobic face of the helix, composed of Val43, Leul47, Phe150, and Leu151, fits snugly into a hydrophobic groove formed by helix H2 and the central beta sheet. At the center of the HDAC9/MEF2 interface, the side chain of Leu147 inserts into a hydrophobic pocket formed by Leu66, Tyr69 and Thr70 of each MEF2 monomer. The long aliphatic side chains of polar resides surrounding Leu147, including Lys144, Lys146 and Gln148, also make extensive van der Waals contacts to MEF2. The interface is largely hydrophobic and the intimate surface complementarity is likely the main source of binding specificity. For residues in HDAC4 found important for MEF2 binding in the biochemical studies described herein (see FIG. 6A-6C and Example 3), such as Val171, Leu175, Phe178, and Val179, their homologous residues in HDAC9, such as Val143, Leu147, Phe150, and Leu151 are located at the binding interface in the crystal structure of the HDAC9/MEF2/DNA complex.

The inventors further analyzed the isolated MEF2-binding motif of HDAC9 for its ability to bind MEF2B (1-93) by ITC. A peptide of 19 residues (SEQ ID NO:22) was synthesized that contains the MEF2-binding motif of HDAC9. In contrast to the recombinant HDAC4 (155-220), which often contains a small amount of impurity despite multi-step purification, the synthetic peptide of HDAC9 can be prepared with high purity. As a result, the ITC data of HDAC9 binding to MEF2B (1-93) were much more accurately measured and well fitted to a simple model of one HDAC9 molecule binding to a preformed MEF2 dimer. The binding stoichiomitry measured by ITC was 0.4760 (HDAC9:MEF2), close to the expected value of 0.5 based on the structure of the HDAC9/MEF2B/DNA complex. The dissociation constant of the HDAC9 peptide/MEF2B (1-93) dimer complex was determined to be 0.72 μM. The high quality of ITC data also allows the inventors to analyze the binding thermodynamics of the HDAC9/MEF2B complex in detail. The free energy of binding of the HDAC9 peptide to MEF2B (1-93) has a relatively small enthalpy component (ΔH=−1.238 kcal/mol) but a significant entropy term (ΔS=23.85 cal/mol.K). This is probably due to the predominantly hydrophobic binding interface observed in the crystal structure. A systematic ITC analysis of the binding of peptides containing the MEF2-binding motifs of other class II HDACs and Cabin1 was hindered by the low solubility of some of these peptides (He, J., Wu, Y., and Han, A., unpublished results).

Discussion

MEF2 and its various co-repressors are often expressed in the same cell. How distinct MEF2/co-repressor complexes function differently is not well understood. The differences in MEF2 binding by distinct MEF2 co-repressors will guide the design of specific MEF2 mutants that disrupt the interaction of one particular MEF2 co-repressor without affecting the others. These mutants will be useful tools for studying the specific function of various MEF2/co-repressor complexes. Other molecular tools for studying the function of MEF2 and for use in therapeutic approaches are peptides that can bind the hydrophobic groove of MEF2 with high affinity and block the binding of a specific (or a subset of) MEF2 co-repressor or co-activator. Such peptides will be especially interesting in cells where the activity of the targeted MEF2/co-regulator complex is aberrantly elevated. Peptides containing the naturally occurring MEF2-binding motif from HDAC9 and Cabin1 bind MEF2 with modest affinity (Kd˜μM). Chimeric peptides combining MEF2-binding residues from different MEF2-co-repressors may bind MEF2 with higher affinities. Alternatively, screening a biased peptide library in which the conserved MEF2-binding residues are fixed may yield a high affinity peptide ligand with a binding surface optimized for MEF2. Recent studies have shown that HDAC inhibitors can suppress hypertrophic gene expression in cardiomyocytes and that MEF2 acts as a key regulator of cytokine gene expression in T cell activation (Zhang et al., Esau et al., Pan et al.). In light of these findings, peptide inhibitors that can block the recruitment of HDACs or co-activators will be key in the development of therapeutic strategies that use MEF2 as a therapeutic target for human diseases involving deregulation of MEF2-dependent genes.

### Example 5

The following example describes the production of an exemplary peptide inhibitor of MEF2.

Based on initial data using an HDAC9 peptide described in Example 4 above, and further based on analysis of the structure of the HDAC9/MEF2/DNA complex, the following peptides were produced:

SEQ ID NO:23:

free NH2 - RAVASTEVKQKLQEFLLSKGSGSYGRKKRRQRRRGC -
amidated

SEQ ID NO:24

free NH2 - RAVASTEDKQKAQEADDSKGSGSYGRKKRRQRRRGC -
amidated

With regard to the peptide represented herein as SEQ ID NO:23, the first 19 amino acid residues correspond to the HDAC9 peptide referenced herein as SEQ ID NO:21, the second 4 amino acids represent a linker peptide, and the C-terminal 13 amino acids are a transducer sequence that will transduce the peptide to the nucleus of a cell.

The peptide of SEQ ID NO:23 binds to MEF2 and modulates the activity of MEF2 in vitro (e.g., by acting as a competitive inhibitor of HDAC9). The peptide of SEQ ID NO:24 is a control peptide that is tranduced to the nucleus of a cell, but does not bind to MEF2. These peptides have the following additional characteristics: (a) they are water soluble; (b) they can be labeled, such as with a fluorescent label; (c) they can efficiently enter the cytoplasm of a cell; (d) they can distribute into the nucleus of a cell.

These peptides are exemplary of inhibitory peptides that can be produced using the information provided by the present invention. Other peptides will be apparent to those of skill in the art upon reviewing these data, such peptides including, for example, the motif represented by SEQ ID NO:22.

Each publication cited herein is incorporated herein by reference in its entirety.

### REFERENCES

1. Youn, H. D., Sun, L., Prywes, R. & Liu, J. O. Apoptosis of T cells mediated by Ca2+-induced release of the transcription factor MEF2. Science 286, 790-3. (1999).

2. Mao, Z., Bonni, A., Xia, F., Nadal-Vicens, M. & Greenberg, M. E. Neuronal activity-dependent cell survival mediated by transcription factor MEF2. Science 286, 785-90. (1999).

3. Black, B. L. & Olson, E. N. Transcriptional control of muscle development by myocyte enhancer factor-2 (MEF2) proteins. Annu Rev Cell Dev Biol 14, 167-96 (1998).

4. McKinsey, T. A., Zhang, C. L. & Olson, E. N. MEF2: a calcium-dependent regulator of cell division, differentiation and death. Trends Biochem Sci 27, 40-7. (2002).

5. Youn, H. D., Grozinger, C. M. & Liu, J. O. Calcium regulates transcriptional repression of myocyte enhancer factor 2 by histone deacetylase 4. J Biol Chem 275, 22563-7. (2000).

6. Zhang, C. L. et al. Class II histone deacetylases act as signal-responsive repressors of cardiac hypertrophy. Cell 110, 479-88. (2002).

7. Youn, H. D. & Liu, J. O. Cabin1 represses MEF2-dependent Nur77 expression and T cell apoptosis by controlling association of histone deacetylases and acetylases with MEF2. Immunity 13, 85-94. (2000).

8. McKinsey, T. A., Zhang, C. L., Lu, J. & Olson, E. N. Signal-dependent nuclear export of a histone deacetylase regulates muscle differentiation [In Process Citation]. Nature 408, 106-11 (2000).

9. Sparrow, D. B. et al. MEF-2 function is modified by a novel co-repressor, MITR. Embo J 18, 5085-98 (1999).

10. Miska, E. A. et al. HDAC4 deacetylase associates with and represses the MEF2 transcription factor. Embo J 18, 5099-107. (1999).

11. Sun, L. et al. Cabin 1, a negative regulator for calcineurin signaling in T lymphocytes. Immunity 8, 703-11. (1998).

12. Lai, M. M., Burnett, P. E., Wolosker, H., Blackshaw, S. & Snyder, S. H. Cain, a novel physiologic protein inhibitor of calcineurin. J Biol Chem 273, 18325-31 (1998).

13. Woronicz, J. D. et al. Regulation of the Nur77 orphan steroid receptor in activation-induced apoptosis. Mol Cell Biol 15, 6364-76 (1995).

14. Santelli, E. & Richmond, T. J. Crystal structure of MEF2A core bound to DNA at 1.5 A resolution. J Mol Biol 297, 437-49. (2000).

15. Huang, K. et al. Solution structure of the MEF2A-DNA complex: structural basis for the modulation of DNA bending and specificity by MADS-box transcription factors. Embo J 19, 2615-28. (2000).

16. Tan, S. & Richmond, T. J. Crystal structure of the yeast MATalpha2/MCM1/DNA ternary complex. Nature 391, 660-6. (1998).

17. Hassler, M. & Richmond, T. J. The B-box dominates SAP-1-SRF interactions in the structure of the ternary complex. Embo J 20, 3018-28 (2001).

18. Pellegrini, L., Tan, S. & Richmond, T. J. Structure of serum response factor core bound to DNA. Nature 376, 490-8. (1995).

19. Molkentin, J. D., Black, B. L., Martin, J. F. & Olson, E. N. Mutational analysis of the DNA binding, dimerization, and transcriptional activation domains of MEF2C. Mol Cell Biol 16, 2627-36. (1996).

20. Molkentin, J. D., Black, B. L., Martin, J. F. & Olson, E. N. Cooperative activation of muscle gene expression by MEF2 and myogenic bHLH proteins. Cell 83, 1125-36. (1995).

21. Bjorkman, P. J. et al. Structure of the human class I histocompatibility antigen, HLA-A2. Nature 329, 506-12 (1987).

22. Lemercier, C. et al. mHDA1/HDAC5 histone deacetylase interacts with and represses MEF2A transcriptional activity. J Biol Chem 275, 15594-9. (2000).

23. Lu, J., McKinsey, T. A., Nicol, R. L. & Olson, E. N. Signal-dependent activation of the MEF2 trascription fact or by dissociation from histone deacetylases. Proc Natl Acad Sci USA 97, 4070-5. (2000).

24. Lu, J., McKinsey, T. A., Zhang, C. L. & Olson, E. N. Regulation of skeletal myogenesis by association of the MEF2 transcription factor with class II histone deacetylases. Mol Cell 6, 233-44. (2000).

25. Sartorelli, V., Huang, J., Hamamori, Y. & Kedes, L. Molecular mechanisms of myogenic coactivation by p300: direct interaction with the activation domain of MyoD and with the MADS box of MEF2C. Mol Cell Biol 17, 1010-26. (1997).

26. Otwinowski, Z. in Proceedings of the CCP4 study weekend (eds. Sawyer, L., Isaacs, N. & Burley, S.) 56-62 (SERC Daresbury Laboratory, Daresbury, UK, 1993).

27. Jones, T. A., Zou, J. Y., Cowan, S. W. & Kjeldgaard. Improved methods for building protein models in electron density maps and the location of errors in these models. Acta Crystallogr A 47 (Pt 2), 110-9 (1991).

28. Brunger, A. T. et al. Crystallography & NMR system: A new software suite for macromolecular structure determination. Acta Crystallogr D Biol Crystallogr 54, 905-21. (1998).

29. The CCP4 suite: Programs for protein crystallography. Acta Crystallogr. D 50, 760-776 (1994).

30. Carson, M. Ribbons 2.0. J. Appl. Crystallogr. 24, 958-961 (1991).

31. Han et al., Nature 422(6933):730-734 (17Apr. 2003); identical to U.S. Provisional Application Ser. No. 60/463,547.

32. Shore et al., Eur J Biochem. April 1;229(1):1-13 (1995).

33. Han et al., J. Mol. Biol. 345, 91-102 (2005).

34. Esau et al. J. Exp. Med. 194, 1449-1459 (2001).

35. Pan et al., J. Biol. Chem. 279, 14477-14480 (2004).

36. Dequiedt et al., Immunity, 18, 687-698 (2003).

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

What is claimed is:

1. An isolated peptide comprising an amino acid sequence represented by SEQ ID NO:22, wherein the peptide is less than about 50 amino acids in length, selectively binds to MEF2, and regulates the activity of MEF2.

2. The isolated peptide of claim 1, wherein the peptide consists essentially of less than 30 amino acids of an amino acid sequence selected from the group consisting of: SEQ ID NO:4, SEQ ID NO:6, SEQ ID NO:8, SEQ ID NO:10, SEQ ID NO:12 and SEQ ID NO:14.

3. The isolated peptide of claim 1, wherein the peptide comprises an amino acid sequence in the first 200-250 N-terminal amino acids of an HDAC.

4. The isolated peptide of claim 1, wherein the peptide comprises an amino acid sequence in the first 160 N-terminal amino acid residues of an HDAC.

5. The isolated peptide of claim 1, wherein the peptide comprises an amino acid sequence comprising or aligning with amino acids represented by any one of SEQ ID NO:18, SEQ ID NO:19 or SEQ ID NO:20.

6. The isolated peptide of claim 1, wherein the peptide comprises an amino acid sequence comprising or aligning with amino acid residues (with respect to SEQ ID NO:4): Val143, Lys144, Lys146, Leu147, Gln148, Phe150 and Leu151.

7. The isolated peptide of claim 1, wherein the peptide comprises an amino acid sequence comprising or aligning with any one or more of amino acids with respect to SEQ ID NO:4: Val143, Lys144, Lys146, Leu147, Glnl48, Phe150, Leu151, and Phe177.

8. The isolated peptide of claim 1, wherein the peptide comprises an amino acid sequence that binds to a region of MEF2 comprising or aligning with any one or more of amino acids (with respect to SEQ ID NO:2): Gln56, Met62, Asp63, Leu66, Leu67, Tyr69, Thr70, Tyr72, Ser73, Glu74, Pro75, and Ser78.

9. The isolated peptide of claim 1, wherein the peptide consists essentially of positions 1-19 of SEQ ID NO:23.

10. The isolated peptide of claim 1, wherein the peptide consists essentially of SEQ ID NO:23.

11. An isolated chimeric peptide comprising the peptide of claim 1 linked to an amino acid sequence for transducing the peptide into the nucleus of a cell.

12. The isolated chimeric peptide of claim 10, wherein the amino acid sequence for transducing the peptide into the nucleus of a cell comprising positions 24-36 of SEQ ID NO:23.

13. The isolated chimeric peptide of claim 1, wherein the chimeric peptide consists essentially of SEQ ID NO:23.

14. A pharmaceutical composition comprising the isolated peptide of claim 1.

15. A pharmaceutical composition comprising the isolated peptide of claim 11.

16. A pharmaceutical composition comprising:
a) a compound having a formula selected from the group consisting of: 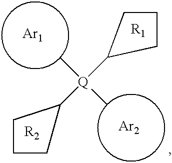
Ar₁—R₂-Q-R₁—Ar₂ and Ar₁—R₁-Q-R₂—Ar₂
or pharmaceutically acceptable salts thereof,
wherein,
Ar₁ and Ar₂ are independently C₅-C₁₀ aromatic, C₅-C₁₀ heterocyclic or aralkyl
R₁ and R₂ are independently C₁-C₁₀ alkyl or alkylene,
Q is C, C═C, C₁-C₁₀ alkyl or phenyl, and
b) a pharmaceutically acceptable carrier.

17. The pharmaceutical composition of claim 16, wherein the compound has the formula: 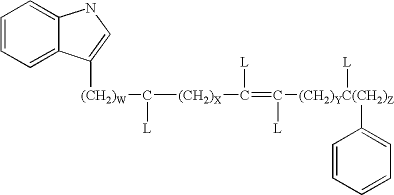
or a pharmaceutically acceptable salt thereof wherein;
each L is independently H, methyl, ethyl, propyl, isopropyl, butyl or isobutyl and W, X, Y and Z are independently integers between 0 and 7.

18. The pharmaceutical composition of claim 16, wherein the compound has the formula: 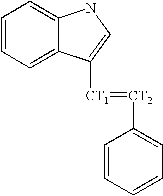
or a pharmaceutically acceptable salt thereof wherein;
T₁ and T₂ are independently butyl, isobutyl, pentane, 2-methylpentane, 3-methylpentane or 4-methylpentane.

19. The pharmaceutical composition of claim 16, wherein the compound has the formula: 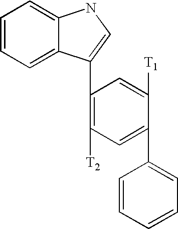
or a pharmaceutically acceptable salt thereof wherein;
T₁ and T₂ are independently propyl, isopropyl, 3-methylbutane or 4-methylbutane.

20. A method of structure-based identification of candidate compounds for regulation of interactions of myocyte enhancer factor 2 (MEF2) with its cognate ligands, comprising:
a) providing a three dimensional structure of an HDAC, a Cabin1 protein or a MEF2 protein in a conformation from a complex of either HDAC or Cabin1 with MEF2 and DNA, the three dimensional structure being selected from the group consisting of:
i) a structure defined by atomic coordinates of a three dimensional structure of a crystalline MEF2 region in complex with DNA and a protein selected from the group consisting of Cabin1 and HDAC9 (MITR);
ii) a structure defined by atomic coordinates selected from the group consisting of:
(1) atomic coordinates represented by a Protein Database Accession No. selected from the group consisting of Protein Database Accession No. 1TQE (HDAC9/MEF2/DNA) and Protein Database Accession No. 1N6J (Cabin1/MEF2/DNA); and,
(2) atomic coordinates that define a three dimensional structure wherein at least 50% of the structure has an average root-mean-square deviation (RMSD) from backbone atoms in secondary structure elements in at least one domain of a three dimensional structure represented by the atomic coordinates of (1) of equal to or less than about 1.5 Å;
iii) a structure defined by atomic coordinates derived from HDAC9/MEF2/DNA molecules arranged in a crystalline manner in a space group P1 so as to form a unit cell of dimensions a=44.765 Å, b=66.859 Å, c=66.924 Å (alpha=76.656, beta=71.846, gamma=71.799);
iv) a structure defined by atomic coordinates derived from Cabin1/MEF2/DNA molecules arranged in a crystalline manner in a space group P4₁22 so as to form a unit cell of dimensions a=b=70.14 Å and c=151.88 Å; and
v) a structure of MEF2 in complex with an HDAC protein and DNA constructed using as a template the three-dimensional structure of (ii);
b) identifying at least one candidate compound for interacting with the three dimensional structure of an active site in MEF2, HDAC, Cabin1, an HDAC and MEF2 complex, or a Cabin1 and MEF2 complex by performing structure based drug design with the structure of (a).


SEQUENCE LISTING (24 sequences)

SEQ ID NO: 1 - DNA, 1098 bp, Homo sapiens
  CDS (1)..(1098)
atggggaggaaaaaaatccagatctcccgcatcctggaccaaaggaatcggcaggtgacgttcaccaagcggaagttcgggctgatgaagaaggcctatgagctgagcgtgctctgtgactgtgagatagccctcatcatcttcaacagcgccaaccgcctcttccagtatgccagcacggacatggaccgtgtgctgctgaagtacacagagtacagcgagccccacgagagccgcaccaacactgacatcctcgagacgctgaagcggaggggcattggcctcgatgggccagagctggagccggatgaagggcctgaggagccaggagagaagtttcggaggctggcaggcgaagggggtgatccggccttgccccgaccccggctgtatcctgcagctcctgctatgcccagcccagatgtggtatacggggccttaccgccaccaggctgtgaccccagtgggcttggggaagcactgcccgcccagagccgcccatctcccttccgaccagcagcccccaaagccgggcccccaggcctggtgcaccctctcttctcaccaagccacctcaccagcaagacaccacccccactgtacctgccgacggaagggcggaggtcagacctgcctggtggcctggctgggccccgagggggactaaacacctccagaagcctctacagtggcctgcagaacccctgctccactgcaactcccggacccccactggggagcttccccttcctccccggaggccccccagtgggggccgaagcctgggcgaggagggtcccccaacccgcggcgcctccccgccgacccccccagtcagcatcaagtctgagcgcctctctccggcccccgggggccccggcgactttcctaagaccttcccctatcccttgctcctcgcccggtccctggcagagcctctgcggcctgggcccgccctgcgccggctgcccttggccgacggctggccccggtaggagatcacccggtggcaccagcccagagcgctcgccaggtacggcgagggcacgtggggaccccacctccctccaggcctcttcagagaagacccaacagtga

SEQ ID NO: 2 - protein, 365 aa, Homo sapiens
MGRKKIQISRILDQRNRQVTFTKRKFGLMKKAYELSVLCDCEIALIIFNSANRLFQYASTDMDRVLLKYTEYSEPHESRTNTDILETLKRRGIGLDGPELEPDEGPEEPGEKFRRLAGEGGDPALPRPRLYPAAPAMPSPDVVYGALPPPGCDPSGLGEALPAQSRPSPFRPAAPKAGPPGLVHPLFSPSHLTSKTPPPLYLPTEGRRSDLPGGLAGPRGGLNTSRSLYSGLQNPCSTATPGPPLGSFPFLPGGPPVGAEAWARRVPQPAAPPRRPPQSASSLSASLRPPGAPATFLRPSPIPCSSPGPWQSLCGLGPPCAGCPWPTAGPGRRSPGGTSPERSPGTARARGDPTSLQASSEKTQQ

SEQ ID NO: 3 - DNA, 1761 bp, Mus musculus
  CDS (1)..(1761)
atgcacagtatgatcagctcagtggacgtgaaatcagaggttcctatgggcctggagcccatctcgcctttagacctgaggacagatctcaggatgatgatgcctgtggtggatcctgtcgtccgcgagaagcaactgcagcaagagttacttcttattcagcagcagcagcagatccagaagcagctcctcatagcagagtttcagaagcagcacgagaatttgacaaggcagcatcaagcacaactccaggagcacatcaaggaacttctagccataaaacaacagcaagaactcctagaaaaggagcagaaactggagcagcagaggcaagaacaggaagtagagaggcatcgcagagagcagcagcttcctcctctcagaggcaaagatagaggacgagaaagggcagtggcaagcacagaggttaagcagaagcttcaagaattcctgttgagtaaatcagcaacaaaagacactccaaccaatggaaagaatcattccgtgggccgccatcccaagctctgggctgcccaccacacatcactggatcaaagctctccacccctcagtggaacgtctccatcctacaagtacacattaccaggagctcaagatagcaaggatgatttccccttgagaaaaactgcctcagagcccaacttgaaggtgcggtccaggttaaaacagaaagtggcagagaggagaagcagtcccttactcaggcggaaggatggaaatcttgtcacttcattcaagaagcgagtgtttgaggtggcagaatcctcggtcagtagcagctctccagggtcaggtcccagttcaccaaacaatggccctgctgggaatgtgaccgaaaatgaggcttcagctctgcctcccacgcctcaccccgagcaactggttccacagcagcgcatactaattcatgaagattccatgaacctgctaagtctctatacctccccgtccctgcccaatatcactctgggacttccagcagtgccgtccccactcaatgcttctaactcactcaaagacaaacagaagtgcgagacacagatgctcagacaaggtgttcctctgcccagtcagtatggcagtagcattgcagcgtcctccagccacgttcatgtagcaatggaaggaaaacccaacagcagccaccaggctctcctgcagcacctactgttgaaggaacaaatgcgacagcaaaagcttctcgtggctggtggagttccattacaccctcagtctcctttggcaacaaaagaaagaatttcaccgggcattagaggtacccacaaattgccccgccaccgacccctgaatcgaacccagtctgcacctttgcctcagagcacgctggctcagctggtcattcagcagcagcaccagcagttcctggagaagcagaaacaataccagcagcagatccacatgaacaaactgctatcgaaatctattgaacaactgaagcaaccaggcagccaccttgaagaagcagaggaggagcttcagggggaccaatccatggaagacagagcagcctctaaggacaccagtgctaggagtgacagcagtgcttgtgtggaggacacgctgggacaagttggggctgtgaaggtcaaggaggaacccgtggacagcgatgaagatgctcagatacaggaaatggaatgtggggagcaggctgcttttatgcaacaggtaataggcaaagatttagctccaggatttgtaattaaagtcattatttga

SEQ ID NO: 4 - protein, 586 aa, Mus musculus
MHSMISSVDVKSEVPMGLEPISPLDLRTDLRMMMPVVDPVVREKQLQQELLLIQQQQQIQKQLLIAEFQKQHENLTRQHQAQLQEHIKELLAIKQQQELLEKEQKLEQQRQEQEVERHRREQQLPPLRGKDRGRERAVASTEVKQKLQEFLLSKSATKDTPTNGKNHSVGRHPKLWAAHHTSLDQSSPPLSGTSPSYKYTLPGAQDSKDDFPLRKTASEPNLKVRSRLKQKVAERRSSPLLRRKDGNLVTSFKKRVFEVAESSVSSSSPGSGPSSPNNGPAGNVTENEASALPPTPHPEQLVPQQRILIHEDSMNLLSLYTSPSLPNITLGLPAVPSPLNASNSLKDKQKCETQMLRQGVPLPSQYGSSIAASSSHVHVAMEGKPNSSHQALLQHLLLKEQMRQQKLLVAGGVPLHPQSPLATKERISPGIRGTHKLPRHRPLNRTQSAPLPQSTLAQLVIQQQHQQFLEKQKQYQQQIHMNKLLSKSIEQLKQPGSHLEEAEEELQGDQSMEDRAASKDTSARSDSSACVEDTLGQVGAVKVKEEPVDSDEDAQIQEMECGEQAAFMQQVIGKDLAPGFVIKVII

SEQ ID NO: 5 - DNA, 3036 bp, Homo sapiens
  CDS (1)..(3036)
atgcacagtatgatcagctcagtggatgtgaagtcagaagttcctgtgggcctggagcccatctcacctttagacctaaggacagacctcaggatgatgatgcccgtggtggaccctgttgtccgtgagaagcaattgcagcaggaattacttcttatccagcagcagcaacaaatccagaagcagcttctgatagcagagtttcagaaacagcatgagaacttgacacggcagcaccaggctcagcttcaggagcatatcaaggaacttctagccataaaacagcaacaagaactcctagaaaaggagcagaaactggagcagcagaggcaagaacaggaagtagagaggcatcgcagagaacagcagcttcctcctctcagaggcaaagatagaggacgagaaagggcagtggcaagtacagaagtaaagcagaagcttcaagagttcctactgagtaaatcagcaacgaaagacactccaactaatggaaaaaatcattccgtgagccgccatcccaagctctggtacacggctgcccaccacacatcattggatcaaagctctccaccccttagtggaacatctccatcctacaagtacacattaccaggagcacaagatgcaaaggatgatttcccccttcgaaaaactgcctctgagcccaacttgaaggtgcggtccaggttaaaacagaaagtggcagagaggagaagcagccccttactcaggcggaaggatggaaatgttgtcacttcattcaagaagcgaatgtttgaggtgacagaatcctcagtcagtagcagttctccaggctctggtcccagttcaccaaacaatgggccaactggaagtgttactgaaaatgagacttcggttttgccccctacccctcatgccgagcaaatggtttcacagcaacgcattctaattcatgaagattccatgaacctgctaagtctttatacctctccttctttgcccaacattaccttggggcttcccgcagtgccatcccagctcaatgcttcgaattcactcaaagaaaagcagaagtgtgagacgcagacgcttaggcaaggtgttcctctgcctgggcagtatggaggcagcatcccggcatcttccagccaccctcatgttactttagagggaaagccacccaacagcagccaccaggctctcctgcagcatttattattgaaagaacaaatgcgacagcaaaagcttcttgtagctggtggagttcccttacatcctcagtctcccttggcaacaaaagagagaatttcacctggcattagaggtacccacaaattgccccgtcacagacccctgaaccgaacccagtctgcacctttgcctcagagcacgttggctcagctggtcattcaacagcaacaccagcaattcttggagaagcagaagcaataccagcagcagatccacatgaacaaactgctttcgaaatctattgaacaactgaagcaaccaggcagtcaccttgaggaagcagaggaagagcttcagggggaccaggcgatgcaggaagacagagcgccctctagtggcaacagcactaggagcgacagcagtgcttgtgtggatgacacactgggacaagttggggctgtgaaggtcaaggaggaaccagtggacagtgatgaagatgctcagatccaggaaatggaatctggggagcaggctgcttttatgcaacagcctttcctggaacccacgcacacacgtgcgctctctgtgcgccaagctccgctggctgcggttggcatggatggattagagaaacaccgtctcgtctccaggactcactcttcccctgctgcctctgttttacctcacccagcaatggaccgccccctccagcctggctctgcaactggaattgcctatgaccccttgatgctgaaacaccagtgcgtttgtggcaattccaccacccaccctgagcatgctggacgaatacagagtatctggtcacgactgcaagaaactgggctgctaaataaatgtgagcgaattcaaggtcgaaaagccagcctggaggaaatacagcttgttcattctgaacatcactcactgttgtatggcaccaaccccctggacggacagaagctggaccccaggatactcctaggtgatgactctcaaaagtttttttcctcattaccttgtggtggacttggggtggacagtgacaccatttggaatgagctacactcgtccggtgctgcacgcatggctgttggctgtgtcatcgagctggcttccaaagtggcctcaggagagctgaagaatgggtttgctgttgtgaggccccctggccatcacgctgaagaatccacagccatggggttctgcttttttaattcagttgcaattaccgccaaatacttgagagaccaactaaatataagcaagatattgattgtagatctggatgttcaccatggaaacggtacccagcaggccttttatgctgaccccagcatcctgtacatttcactccatcgctatgatgaagggaactttttccctggcagtggagccccaaatgaggttggaacaggccttggagaagggtacaatataaatattgcctggacaggtggccttgatcctcccatgggagatgttgagtaccttgaagcattcaggaccatcgtgaagcctgtggccaaagagtttgatccagacatggtcttagtatctgctggatttgatgcattggaaggccacacccctcctctaggagggtacaaagtgacggcaaaatgttttggtcatttgacgaagcaattgatgacattggctgatggacgtgtggtgttggctctagaaggaggacatgatctcacagccatctgtgatgcatcagaagcctgtgtaaatgcccttctaggaaatgagctggagccacttgcagaagatattctccaccaaagcccgaatatgaatgctgttatttctttacagaagatcattgaaattcaaagtatgtctttaaagttctcttaa

SEQ ID NO: 6 - protein, 1011 aa, Homo sapiens
MHSMISSVDVKSEVPVGLEPISPLDLRTDLRMMMPVVDPVVREKQLQQELLLIQQQQQIQKQLLIAEFQKQHENLTRQHQAQLQEHIKELLAIKQQQELLEKEQKLEQQRQEQEVERHRREQQLPPLRGKDRGRERAVASTEVKQKLQEFLLSKSATKDTPTNGKNHSVSRHPKLWYTAAHHTSLDQSSPPLSGTSPSYKYTLPGAQDAKDDFPLRKTASEPNLKVRSRLKQKVAERRSSPLLRRKDGNVVTSFKKRMFEVTESSVSSSSPGSGPSSPNNGPTGSVTENETSVLPPTPHAEQMVSQQRILIHEDSMNLLSLYTSPSLPNITLGLPAVPSQLNASNSLKEKQKCETQTLRQGVPLPGQYGGSIPASSSHPHVTLEGKPPNSSHQALLQHLLLKEQMRQQKLLVAGGVPLHPQSPLATKERISPGIRGTHKLPRHRPLNRTQSAPLPQSTLAQLVIQQQHQQFLEKQKQYQQQIHMNKLLSKSIEQLKQPGSHLEEAEEELQGDQAMQEDRAPSSGNSTRSDSSACVDDTLGQVGAVKVKEEPVDSDEDAQIQEMESGEQAAFMQQPFLEPTHTRALSVRQAPLAAVGMDGLEKHRLVSRTHSSPAASVLPHPAMDRPLQPGSATGIAYDPLMLKHQCVCGNSTTHPEHAGRIQSIWSRLQETGLLNKCERIQGRKASLEEIQLVHSEHHSLLYGTNPLDGQKLDPRILLGDDSQKFFSSLPCGGLGVDSDTIWNELHSSGAARMAVGCVIELASKVASGELKNGFAVVRPPGHHAEESTAMGFCFFNSVAITAKYLRDQLNISKILIVDLDVHHGNGTQQAFYADPSILYISLHRYDEGNFFPGSGAPNEVGTGLGEGYNINIAWTGGLDPPMGDVEYLEAFRTIVKPVAKEFDPDMVLVSAGFDALEGHTPPLGGYKVTAKCFGHLTKQLMTLADGRVVLALEGGHDLTAICDASEACVNALLGNELEPLAEDILHQSPNMNAVISLQKIIEIQSMSLKFS

SEQ ID NO: 7 - DNA, 3255 bp, Homo sapiens
  CDS (1)..(3255)
atgagctcccaaagccatccagatggactttctggccgagaccagccagtggagctgctgaatcctgcccgcgtgaaccacatgcccagcacggtggatgtggccacggcgctgcctctgcaagtggccccctcggcagtgcccatggacctgcgcctggaccaccagttctcactgcctgtggcagagccggccctgcgggagcagcagctgcagcaggagctcctggcgctcaagcagaagcagcagatccagaggcagatcctcatcgctgagttccagaggcagcacgagcagctctcccggcagcacgaggcgcagctccacgagcacatcaagcaacaacaggagatgctggccatgaagcaccagcaggagctgctggaacaccagcggaagctggagaggcaccgccaggagcaggagctggagaagcagcaccgggagcagaagctgcagcagctcaagaacaaggagaagggcaaagagagtgccgtggccagcacagaagtgaagatgaagttacaagaatttgtcctcaataaaaagaaggcgctggcccaccggaatctgaaccactgcatttccagcgaccctcgctactggtacgggaaaacgcagcacagttcccttgaccagagttctccaccccagagcggagtgtcgacctcctataaccacccggtcctgggaatgtacgacgccaaagatgacttccctcttaggaaaacagcttctgaaccgaatctgaaattacggtccaggctaaagcagaaagtggccgaaagacggagcagccccctgttacgcaggaaagacgggccagtggtcactgctctaaaaaagcgtccgttggatgtcacagactccgcgtgcagcagcgccccaggctccggacccagctcacccaacaacagctccgggagcgtcagcgcggagaacggtatcgcgcccgccgtccccagcatcccggcggagacgagtttggcgcacagacttgtggcacgagaaggctcggccgctccacttcccctctacacatcgccatccttgcccaacatcacgctgggcctgcctgccaccggcccctctgcgggcacggcgggccagcaggacaccgagagactcacccttcccgccctccagcagaggctctcccttttccccggcacccacctcactccctacctgagcacctcgcccttggagcgggacggaggggcagcgcacagccctcttctgcagcacatggtcttactggagcagccaccggcacaagcacccctcgtcacaggcctgggagcactgcccctccacgcacagtccttggttggtgcagaccgggtgtccccctccatccacaagctgcggcagcaccgcccactggggcggacccagtcggccccgctgccccagaacgcccaggctctgcagcacctggtcatccagcagcagcatcagcagtttctggagaaacacaagcagcagttccagcagcagcaactgcagatgaacaagatcatccccaagccaagcgagccagcccggcagccggagagccacccggaggagacggaggaggagctccgtgagcaccaggctctgctggacgagccctacctggaccggctgccggggcagaaggaggcgcacgcacaggccggcgtgcaggtgaagcaggagcccattgagagcgatgaggaagaggcagagcccccacgggaggtggagccgggccagcgccagcccagtgagcaggagctgctcttcagacagcaagccctcctgctggagcagcagcggatccaccagctgaggaactaccaggcgtccatggaggccgccggcatccccgtgtccttcggcggccacaggcctctgtcccgggcgcagtcctcacccgcgtctgccaccttccccgtgtctgtgcaggagccccccaccaagccgaggttcacgacaggcctcgtgtatgacacgctgatgctgaagcaccagtgcacctgcgggagtagcagcagccaccccgagcacgccgggaggatccagagcatctggtcccgcctgcaggagacgggcctccggggcaaatgcgagtgcatccgcggacgcaaggccaccctggaggagctacagacggtgcactcggaagcccacaccctcctgtatggcacgaaccccctcaaccggcagaaactggacagtaagaaacttctaggctcgctcgcctccgtgttcgtccggctcccttgcggtggtgttggggtggacagtgacaccatatggaacgaggtgcactcggcgggggcagcccgcctggctgtgggctgcgtggtagagctggtcttcaaggtggccacaggggagctgaagaatggctttgctgtggtccgcccccctggacaccatgcggaggagagcacgcccatgggcttttgctacttcaactccgtggccgtggcagccaagcttctgcagcagaggttgagcgtgagcaagatcctcatcgtggactgggacgtgcaccatggaaacgggacccagcaggctttctacagcgaccctagcgtcctgtacatgtccctccaccgctacgacgatgggaacttcttcccaggcagcggggctcctgatgaggtgggcacagggcccggcgtgggtttcaacgtcaacatggctttcaccggcggcctggacccccccatgggagacgctgagtacttggcggccttcagaacggtggtcatgccgatcgccagcgagtttgccccggatgtggtgctggtgtcatcaggcttcgatgccgtggagggccaccccacccctcttgggggctacaacctctccgccagatgcttcgggtacctgacgaagcagctgatgggcctggctggcggccggattgtcctggccctcgagggaggccacgacctgaccgccatttgcgacgcctcggaagcatgtgtttctgccttgctgggaaacgagcttgatcctctcccagaaaaggttttacagcaaagacccaatgcaaacgctgtccgttccatggagaaagtcatggagatccacagcaagtactggcgctgcctgcagcgcacaacctccacagcggggcgttctctgatcgaggctcagacttgcgagaacgaagaagccgagacggtcaccgccatggcctcgctgtccgtgggcgtgaagcccgccgaaaagagaccagatgaggagcccatggaagaggagccgcccctgtag

SEQ ID NO: 8 - protein, 1084 aa, Homo sapiens
MSSQSHPDGLSGRDQPVELLNPARVNHMPSTVDVATALPLQVAPSAVPMDLRLDHQFSLPVAEPALREQQLQQELLALKQKQQIQRQILIAEFQRQHEQLSRQHEAQLHEHIKQQQEMLAMKHQQELLEHQRKLERHRQEQELEKQHREQKLQQLKNKEKGKESAVASTEVKMKLQEFVLNKKKALAHRNLNHCISSDPRYWYGKTQHSSLDQSSPPQSGVSTSYNHPVLGMYDAKDDFPLRKTASEPNLKLRSRLKQKVAERRSSPLLRRKDGPVVTALKKRPLDVTDSACSSAPGSGPSSPNNSSGSVSAENGIAPAVPSIPAETSLAHRLVAREGSAAPLPLYTSPSLPNITLGLPATGPSAGTAGQQDTERLTLPALQQRLSLFPGTHLTPYLSTSPLERDGGAAHSPLLQHMVLLEQPPAQAPLVTGLGALPLHAQSLVGADRVSPSIHKLRQHRPLGRTQSAPLPQNAQALQHLVIQQQHQQFLEKHKQQFQQQQLQMNKIIPKPSEPARQPESHPEETEEELREHQALLDEPYLDRLPGQKEAHAQAGVQVKQEPIESDEEEAEPPREVEPGQRQPSEQELLFRQQALLLEQQRIHQLRNYQASMEAAGIPVSFGGHRPLSRAQSSPASATFPVSVQEPPTKPRFTTGLVYDTLMLKHQCTCGSSSSHPEHAGRIQSIWSRLQETGLRGKCECIRGRKATLEELQTVHSEAHTLLYGTNPLNRQKLDSKKLLGSLASVFVRLPCGGVGVDSDTIWNEVHSAGAARLAVGCVVELVFKVATGELKNGFAVVRPPGHHAEESTPMGFCYFNSVAVAAKLLQQRLSVSKILIVDWDVHHGNGTQQAFYSDPSVLYMSLHRYDDGNFFPGSGAPDEVGTGPGVGFNVNMAFTGGLDPPMGDAEYLAAFRTVVMPIASEFAPDVVLVSSGFDAVEGHPTPLGGYNLSARCFGYLTKQLMGLAGGRIVLALEGGHDLTAICDASEACVSALLGNELDPLPEKVLQQRPNANAVRSMEKVMEIHSKYWRCLQRTTSTAGRSLIEAQTCENEEAETVTAMASLSVGVKPAEKRPDEEPMEEEPPL

SEQ ID NO: 9 - DNA, 3369 bp, Homo sapiens
  CDS (1)..(3369)
atgaactctcccaacgagtcggatgggatgtcaggtcgggaaccatccttggaaatcctgccgcggacttctctgcacagcatccctgtgacagtggaggtgaagccggtgctgccaagagccatgcccagttccatggggggtgggggtggaggcagccccagccctgtggagctacggggggctctggtgggctctgtggaccccacactgcgggagcagcaactgcagcaggagctcctggcgctcaagcagcagcagcagctgcagaagcagctcctgttcgctgagttccagaaacagcatgaccacctgacaaggcagcatgaggtccagctgcagaagcacctcaagcagcagcaggagatgctggcagccaagcagcagcaggagatgctggcagccaagcggcagcaggagctggagcagcagcggcagcgggagcagcagcggcaggaagagctggagaagcagcggctggagcagcagctgctcatcctgcggaacaaggagaagagcaaagagagtgccattgccagcactgaggtaaagctgaggctccaggaattcctcttgtcgaagtcaaaggagcccacaccaggcggcctcaaccattccctcccacagcaccccaaatgctggggagcccaccatgcttctttggaccagagttcccctccccagagcggcccccctgggacgcctccctcctacaaactgcctttgcctgggccctacgacagtcgagacgacttccccctccgcaaaacagcctctgaacccaacttgaaagtgcgttcaaggctaaaacagaaggtggctgagcggagaagcagtcccctcctgcgtcgcaaggatgggactgttattagcacctttaagaagagagctgttgagatcacaggtgccgggcctggggcgtcgtccgtgtgtaacagcgcacccggctccggccccagctctcccaacagctcccacagcaccatcgctgagaatggctttactggctcagtccccaacatccccactgagatgctccctcagcaccgagccctccctctggacagctcccccaaccagttcagcctctacacgtctccttctctgcccaacatctccctagggctgcaggccacggtcactgtcaccaactcacacctcactgcctccccgaagctgtcgacacagcaggaggccgagaggcaggccctccagtccctgcggcagggtggcacgctgaccggcaagttcatgagcacatcctctattcctggctgcctgctgggcgtggcactggagggcgacgggagcccccacgggcatgcctccctgctgcagcatgtgctgttgctggagcaggcccggcagcagagcaccctcattgctgtgccactccacgggcagtccccactagtgacgggtgaacgtgtggccaccagcatgcggacggtaggcaagctcccgcggcatcggcccctgagccgcactcagtcctcaccgctgccgcagagtccccaggccctgcagcagctggtcatgcaacaacagcaccagcagttcctggagaagcagaagcagcagcagctacagctgggcaagatcctcaccaagacaggggagctgcccaggcagcccaccacccaccctgaggagacagaggaggagctgacggagcagcaggaggtcttgctgggggagggagccctgaccatgccccgggagggctccacagagagtgagagcacacaggaagacctggaggaggaggacgaggaagaggatggggaggaggaggaggattgcatccaggttaaggacgaggagggcgagagtggtgctgaggaggggcccgacttggaggagcctggtgctggatacaaaaaactgttctcagatgcccagccgctgcagcctttgcaggtgtaccaggcgcccctcagcctggccactgtgccccaccaggccctgggccgtacccagtcctcccctgctgcccctgggggcatgaagagccccccagaccagcccgtcaagcacctcttcaccacaggtgtggtctacgacacgttcatgctaaagcaccagtgcatgtgcgggaacacacacgtgcaccctgagcatgctggccggatccagagcatctggtcccggctgcaggagacaggcctgcttagcaagtgcgagcggatccgaggtcgcaaagccacgctagatgagatccagacagtgcactctgaataccacaccctgctctatgggaccagtcccctcaaccggcagaagctagacagcaagaagttgctcggccccatcagccagaagatgtatgctgtgctgccttgtgggggcatcggggtggacagtgacaccgtgtggaatgagatgcactcctccagtgctgtgcgcatggcagtgggctgcctgctggagctggccttcaaggtggctgcaggagagctcaagaatggatttgccatcatccggcccccaggacaccacgccgaggaatccacagccatgggattctgcttcttcaactctgtagccatcaccgcaaaactcctacagcagaagttgaacgtgggcaaggtcctcatcgtggactgggacattcaccatggcaatggcacccagcaggcgttctataatgacccctctgtgctctacatctctctgcatcgctatgacaacgggaacttctttccaggctctggggctcctgaagaggttggtggaggaccaggcgtggggtacaatgtgaacgtggcatggacaggaggtgtggacccccccattggagacgtggagtaccttacagccttcaggacagtggtgatgcccattgcccacgagttctcacctgatgtggtcctagtctccgccgggtttgatgctgttgaaggacatctgtctcctctgggtggctactctgtcaccgccagatgttttggccacttgaccaggcagctgatgaccctggcagggggccgggtggtgctggccctggagggaggccatgacttgaccgccatctgtgatgcctctgaggcttgtgtctcggctctgctcagtgtagagctgcagcccttggatgaggcagtcttgcagcaaaagcccaacatcaacgcagtggccacgctagagaaagtcatcgagatccagagcaaacactggagctgtgtgcagaagttcgccgctggtctgggccggtccctgcgagaggcccaagcaggtgagaccgaggaggccgagactgtgagcgccatggccttgctgtcggtgggggccgagcaggcccaggctgcggcagcccgggaacacagccccaggccggcagaggagcccatggagcaggagcctgccctgtga

SEQ ID NO: 10 - protein, 1122 aa, Homo sapiens
MNSPNESDGMSGREPSLEILPRTSLHSIPVTVEVKPVLPRAMPSSMGGGGGGSPSPVELRGALVGSVDPTLREQQLQQELLALKQQQQLQKQLLFAEFQKQHDHLTRQHEVQLQKHLKQQQEMLAAKQQQEMLAAKRQQELEQQRQREQQRQEELEKQRLEQQLLILRNKEKSKESAIASTEVKLRLQEFLLSKSKEPTPGGLNHSLPQHPKCWGAHHASLDQSSPPQSGPPGTPPSYKLPLPGPYDSRDDFPLRKTASEPNLKVRSRLKQKVAERRSSPLLRRKDGTVISTFKKRAVEITGAGPGASSVCNSAPGSGPSSPNSSHSTIAENGFTGSVPNIPTEMLPQHRALPLDSSPNQFSLYTSPSLPNISLGLQATVTVTNSHLTASPKLSTQQEAERQALQSLRQGGTLTGKFMSTSSIPGCLLGVALEGDGSPHGHASLLQHVLLLEQARQQSTLIAVPLHGQSPLVTGERVATSMRTVGKLPRHRPLSRTQSSPLPQSPQALQQLVMQQQHQQFLEKQKQQQLQLGKILTKTGELPRQPTTHPEETEEELTEQQEVLLGEGALTMPREGSTESESTQEDLEEEDEEEDGEEEEDCIQVKDEEGESGAEEGPDLEEPGAGYKKLFSDAQPLQPLQVYQAPLSLATVPHQALGRTQSSPAAPGGMKSPPDQPVKHLFTTGVVYDTFMLKHQCMCGNTHVHPEHAGRIQSIWSRLQETGLLSKCERIRGRKATLDEIQTVHSEYHTLLYGTSPLNRQKLDSKKLLGPISQKMYAVLPCGGIGVDSDTVWNEMHSSSAVRMAVGCLLELAFKVAAGELKNGFAIIRPPGHHAEESTAMGFCFFNSVAITAKLLQQKLNVGKVLIVDWDIHHGNGTQQAFYNDPSVLYISLHRYDNGNFFPGSGAPEEVGGGPGVGYNVNVAWTGGVDPPIGDVEYLTAFRTVVMPIAHEFSPDVVLVSAGFDAVEGHLSPLGGYSVTARCFGHLTRQLMTLAGGRVVLALEGGHDLTAICDASEACVSALLSVELQPLDEAVLQQKPNINAVATLEKVIEIQSKHWSCVQKFAAGLGRSLREAQAGETEEAETVSAMALLSVGAEQAQAAAAREHSPRPAEEPMEQEPAL

SEQ ID NO: 11 - DNA, 831 bp, Homo sapiens
  CDS (1)..(831)
atgggcttctgcttcttcaactcagtggccatcgcctgccggcagctgcaacagcagagcaaggccagcaagatcctcattgtagactgggacgtgcaccatggcaacggcacccagcaaaccttctaccaagaccccagtgtgctctacatctccctgcatcgccatgacgacggcaacttcttcccggggagtggggctgtggatgaggtaggggctggcagcggtgagggcttcaatgtcaatgtggcctgggctggaggtctggacccccccatgggggatcctgagtacctggctgctttcaggatagtcgtgatgcccatcgcccgagagttctctccagacctagtcctggtgtctgctggatttgatgctgctgagggtcacccggccccactgggtggctaccatgtttctgccaaatgttttggatacatgacgcagcaactgatgaacctggcaggaggcgcagtggtgctggccttggagggtggccatgacctcacagccatctgtgacgcctctgaggcctgtgtggctgctcttctgggtaacagggtggatcccctttcagaagaaggctggaaacagaaacccaacctcaatgccatccgctctctggaggccgtgatccgggtgcacagtaaatactggggctgcatgcagcgcctggcctcctgtccagactcctgggtgcctagagtgccaggggctgacaaagaagaagtggaggcagtgaccgcactggcgtccctctctgtgggcatcctggctgaagataggccctcggagcagctggtggaggaggaagaacctatgaatctctaa

SEQ ID NO: 12 - protein, 276 aa, Homo sapiens [sequence omitted]

SEQ ID NO: 13 - DNA, 6663 bp, Homo sapiens
  CDS (1)..(6663)
atgattcgaattgcagccttaaatgccagctccaccattgaggatgatcatgaaggaagctttaaaagtcacaaaacccagacaaaggaggctcaggaagcagaggcttttgcattgtaccacaaggcccttgatctgcagaaacatgaccggtttgaggagtctgccaaagcctaccatgagctcttggaggcgagcctgctgcgggaggcagtttcatccggtgatgagaaagaggggttgaaacaccctgggctgatactgaaatattccacttataagaacttggcccagctggcagcccagcgggaggatctggagacagccatggagttctacttagaggcagtgatgctggactccacagatgtcaacctctggtataagattggacatgtggccctgaggctcatccggatccccctggctcgccatgcttttgaggaagggctgcggtgcaatcctgaccactggccctgtttggataacctaatcactgtcctgtacaccctcagtgattacacaacatgtctgtacttcatctgcaaagctttggagaaggattgccggtacagcaaagggctggtcctcaaggagaagatttttgaggagcagccttgtctccggaaggactctctcagaatgttcctcaaatgtgacatgtcgattcacgatgtttcggtgagtgcagctgagacacaggcgattgtagatgaggccttggggctgcgaaaaaagaggcaagcgctgattgtgcgggagaaggagccggacctgaaacttgtgcagcccattcctttcttcacctggaagtgcctcggagagagcttgctggccatgtacaatcatctcaccacctgtgagcccccacgtcccagccttggcaaaaggattgatttgtcggactaccaggaccccagccagcctcttgagtcctccatggtggtgacgccagttaacgtgatccagccaagcactgtcagcaccaacccagctgtggctgtcgccgagcctgtggtctcctacacctctgtggctacaaccagcttcccactgcacagtcctggtctgttggagacaggcgctcctgtgggtgatatttctgggggagataaatccaagaaaggggtaaaacggaagaagatttcagaagagagtggagaaacagcaaagcggcggtctgcccgtgtccgaaacaccaagtgcaaaaaagaagagaaagtagacttccaggagcttctgatgaagttcttgccgtccaggttaagaaagctggaccctgaggaggaagatgattcctttaataactatgaagtccagtcagaagccaaactggaaagcttcccaagcattgggcctcaaaggctgtcatttgactcagccacattcatggaatctgaaaagcaggacgtgcatgagttcctgctggagaacctaaccaacgggggcatcctggagctgatgatgcgctacctgaaagccatgggccacaagttcttggtaaggtggcctccaggcttggcggaggtcgtgctcagcgtctaccacagctggaggaggcacagcaccagcctgcccaacccgctgctgagggactgcagcaacaagcacatcaaggacatgatgctgatgtctctctcctgcatggaactccagctggaccagtggctgctgaccaaaggcagaagctctgcagtgtctcctcggaactgccctgctggtatggtgaatggcagatttggacctgacttcccagggacccactgcctgggtgacctcctacagctgtcatttgcctcgtcccagcgcgacctgttcgaggatggttggctggagtttgtggtccgtgtttactggctgaaggctcgcttcctggcgctgcagggagacatggagcaggccctggagaactatgacatctgcacagaaatgctccagagttccaccgccatccaggtggaggcaggggctgaacgaagagacattgtcatccggctgcccaacctccataatgactctgtggtttccctggaggagattgataagaacctgaagtcgctggagcggtgccagtccctggaggagattcagcggctgtatgaagcaggcgactacaaggctgttgtgcatctgctccgccccactttgtgcaccagtgggtttgaccgggccaaacacctggagtttatgacttccattcctgagaggccagcccagctgcttcttctgcaggactccttgctccggctgaaggactatcggcagtgttttgagtgttccgatgtggctctgaacgaggctgtccagcagatggtgaactcaggtgaggctgccgccaaggaggagtgggtggccacagtgacccaactgctgatgggcatcgagcaggccctctctgcggacagcagtggtagcatcctgaaggtatcatcctccaccactggccttgtgcggctcaccaacaacctcatccaggtcattgactgcagcatggctgtgcaggaggaggccaaggagccccacgtctcttcagtgctaccctggatcattctacaccggatcatctggcaggaggaagacaccttccattctctgtgccaccagcagcagctccaaaacccagcggaggaagggatgtcagagacgcccatgctcccatcctccctcatgctgctgaacacagcccacgagtatttgggcagaaggtcctggtgctgcaattcagatggggctctgctgcgattctatgtgcgagtactccagaaggaactggctgcatccacctctgaagacacgcacccttacaaggaggagctggagacagccttggagcagtgcttctactgcctgtacagcttccccagcaagaagagtaaggccaggtacctggaggaacactcggcccagcaggtggatcttatatgggaggatgcactgttcatgtttgagtattttaagcccaagacccttcctgaatttgacagctataagaccagcaccgtgtctgctgacttggccaacctactgaagagaattgccaccattgtgcctcgcacagagaggccagcccttagcctggacaaagtctctgcctacattgagggaacttcaactgaggtaccctgcctcccagagggggctgacccctcccctccagtggtgaacgagctttactacctcctggctgattatcatttcaaaaacaaggagcagtccaaggccatcaagttctacatgcatgacatctgcatctgccccaataggtttgattcctgggcaggcatggctctggcccgggccagccgcattcaggacaagctgaactccaatgagctgaagagtgatgggcccatttggaagcatgccacgcccgtcttgaactgcttccgtcgggccctggagattgacagctccaacttgtccctatggattgagtatggcaccatgtcctatgccttgcactcattcgcctcacgtcaattgaagcagtggagaggcgagctgccccctgagctcgtgcagcagatggagggccggcgcgacagcatgctagagacagccaagcactgtttcacatcagcagcccgctgcgagggtgatggtgacgaggaggagtggctcatccactacatgctgggcaaggtggctgagaagcagcagcagccacccaccgtttacttgctgcactacaggcaggctggccactacctgcacgaggaggctgcccgctaccccaagaagatccactaccacaacccacctgagctggccatggaggccctggaggtgtactttcggctccatgcttccatcctgaagctcctggggaagcccgattctggggttggtgcagaggtcctggtcaactttatgaaggaggctgcagaaggaccctttgccaggggcgaggagaagaacacacccaaagcttcagaaaaggagaaggcctgcctggtggacgaggactcccactcttcagctgggacactgccgggccccggagcctccctcccctcctcctctggcccaggtctgacatccccaccttacacagccactccgattgaccacgattacgtcaaatgtaaaaaaccccaccagcaggcaacgccggacgaccgaagccaggacagcacagccgtagcactctcagactctagctcaacgcaggacttctttaatgagcccaccagcttactggaaggctccaggaaatcctacacagagaagaggctgcccattctcagttcccaagcaggagcgacgggtaaagatcttcagggggccacagaagaaagaggaaaaaacgaggagtcattggagagtacagaaggcttccgggctgcagagcaaggtgtccagaagcctgctgcagaaaccccagcctctgcttgcatccctggcaagccctcagcatccacacccaccctgtgggatgggaagaagagaggggacctcccaggggagccagtggccttcccccaggggctgccggctggtgctgaggagcagcggcagtttctcacagagcagtgcatcgcctccttccgcctgtgcctgagccgcttcccccagcactataagagtctctaccgtctggccttcctctacacctacagcaagacccaccggaacctccagtgggcccgcgacgtgttgctaggcagcagtatcccgtggcaacaactgcagcacatgccggcacaggggctcttctgcgagaggaacaagaccaatttcttcaacggcatctggcggatccccgtggacgagattgaccggccgggcagctttgcctggcacatgaaccgctccatcgtgctgctgctcaaggtgctggcccagctgcgggaccacagcaccctgctgaaggtgtcctccatgcttcagcggaccccagaccagggcaagaagtatctgcgagatgctgaccgccaggtcctggcgcagcgggccttcatcctcactgtgaaggtgctcgaagacacgctgagcgagctcgcagaggggtcagaacgcccagggcccaaggtctgtggcctccccggagccaggatgaccaccgatgtctcacacaaggccagtcctgaggatggccaggagggcctcccccagccgaagaagccccctctggctgatggctcagggccagggcccgagccaggaggcaaagtgggcctcctcaaccaccggcctgtggccatggatgcaggagacagtgcagaccaaagcggggagcggaaggataaagagagcccacgggcagggcccactgagcccatggacacgagtgaggccactgtttgccactcagacttggagcggacaccacccctgctgccaggtcgccccgcaagggaccggggccccgagagccggcccactgagctgtccctggaggagctgagcatcagtgcccggcagcagcccaccccgctcaccccagcccagccagcccccgcccccgcccccgccaccaccacagggaccagggcagggggccacccggaggagccgctctcccggctcagccgcaagaggaagctcctggaggacacagagtcaggcaagacacttctgttggatgcctaccgtgtgtggcagcagggccagaagggtgtggcctatgacctgggccgtgtggagaggatcatgtcggagacctacatgctcatcaagcaggtggatgaggaggctgcgctggagcaggctgtgaagttctgccaggtccatcttggggctgccgcccagagacaggcctcgggggacacccccaccactccaaagcaccccaaagacagccgagagaacttctttcctgtgacagtggtgcccacagcccctgaccctgtgccagctgactctgtccagcggcccagtgatgctcacaccaagcctcgccctgcactagctgccgccacaactattatcacctgccctccgtcagcatcagcttccaccctggaccagtccaaggaccctgggcctccccggccacacaggcctgaagctacccccagcatggcctctctgggcccagagggagaagagctggcgagagtggcagagggcaccagcttcccgcctcaggagccacggcacagtccgcaggtgaagatggcccccacaagttccccggcagagccacactgctggccggcagaggctgccctgggcacaggcgctgagcccacctgcagccaggaggggaaactgaggcctgagccgagaagggatggggaggctcaggaggctgcgagtgagactcagcccctgagctctcccccaacagctgccagctccaaggcccccagcagtgggagtgcccagccaccagagggtcacccaggcaagcctgagcccagccgggctaagtcccgccccctgcccaacatgccaaagctggtcatcccctccgccgccaccaagttcccccctgagatcaccgtcacgccacccaccccaaccctgctctcccccaaaggcagcatctcggaggagaccaagcagaagctgaagtcagccatcctttctgcccagtctgctgccaacgtgaggaaggagagcctatgccagccagccctggaggtcctggagacatccagccaggagtcctcgctggagagcgagacagacgaggacgacgactacatggacatttga

SEQ ID NO: 14 - protein, 2220 aa, Homo sapiens
MIRIAALNASSTIEDDHEGSFKSHKTQTKEAQEAEAFALYHKALDLQKHDRFEESAKAYHELLEASLLREAVSSGDEKEGLKHPGLILKYSTYKNLAQLAAQREDLETAMEFYLEAVMLDSTDVNLWYKIGHVALRLIRIPLARHAFEEGLRCNPDHWPCLDNLITVLYTLSDYTTCLYFICKALEKDCRYSKGLVLKEKIFEEQPCLRKDSLRMFLKCDMSIHDVSVSAAETQAIVDEALGLRKKRQALIVREKEPDLKLVQPIPFFTWKCLGESLLAMYNHLTTCEPPRPSLGKRIDLSDYQDPSQPLESSMVVTPVNVIQPSTVSTNPAVAVAEPVVSYTSVATTSFPLHSPGLLETGAPVGDISGGDKSKKGVKRKKISEESGETAKRRSARVRNTKCKKEEKVDFQELLMKFLPSRLRKLDPEEEDDSFNNYEVQSEAKLESFPSIGPQRLSFDSATFMESEKQDVHEFLLENLTNGGILELMMRYLKAMGHKFLVRWPPGLAEVVLSVYHSWRRHSTSLPNPLLRDCSNKHIKDMMLMSLSCMELQLDQWLLTKGRSSAVSPRNCPAGMVNGRFGPDFPGTHCLGDLLQLSFASSQRDLFEDGWLEFVVRVYWLKARFLALQGDMEQALENYDICTEMLQSSTAIQVEAGAERRDIVIRLPNLHNDSVVSLEEIDKNLKSLERCQSLEEIQRLYEAGDYKAVVHLLRPTLCTSGFDRAKHLEFMTSIPERPAQLLLLQDSLLRLKDYRQCFECSDVALNEAVQQMVNSGEAAAKEEWVATVTQLLMGIEQALSADSSGSILKVSSSTTGLVRLTNNLIQVIDCSMAVQEEAKEPHVSSVLPWIILHRIIWQEEDTFHSLCHQQQLQNPAEEGMSETPMLPSSLMLLNTAHEYLGRRSWCCNSDGALLRFYVRVLQKELAASTSEDTHPYKEELETALEQCFYCLYSFPSKKSKARYLEEHSAQQVDLIWEDALFMFEYFKPKTLPEFDSYKTSTVSADLANLLKRIATIVPRTERPALSLDKVSAYIEGTSTEVPCLPEGADPSPPVVNELYYLLADYHFKNKEQSKAIKFYMHDICICPNRFDSWAGMALARASRIQDKLNSNELKSDGPIWKHATPVLNCFRRALEIDSSNLSLWIEYGTMSYALHSFASRQLKQWRGELPPELVQQMEGRRDSMLETAKHCFTSAARCEGDGDEEEWLIHYMLGKVAEKQQQPPTVYLLHYRQAGHYLHEEAARYPKKIHYHNPPELAMEALEVYFRLHASILKLLGKPDSGVGAEVLVNFMKEAAEGPFARGEEKNTPKASEKEKACLVDEDSHSSAGTLPGPGASLPSSSGPGLTSPPYTATPIDHDYVKCKKPHQQATPDDRSQDSTAVALSDSSSTQDFFNEPTSLLEGSRKSYTEKRLPILSSQAGATGKDLQGATEERGKNEESLESTEGFRAAEQGVQKPAAETPASACIPGKPSASTPTLWDGKKRGDLPGEPVAFPQGLPAGAEEQRQFLTEQCIASFRLCLSRFPQHYKSLYRLAFLYTYSKTHRNLQWARDVLLGSSIPWQQLQHMPAQGLFCERNKTNFFNGIWRIPVDEIDRPGSFAWHMNRSIVLLLKVLAQLRDHSTLLKVSSMLQRTPDQGKKYLRDADRQVLAQRAFILTVKVLEDTLSELAEGSERPGPKVCGLPGARMTTDVSHKASPEDGQEGLPQPKKPPLADGSGPGPEPGGKVGLLNHRPVAMDAGDSADQSGERKDKESPRAGPTEPMDTSEATVCHSDLERTPPLLPGRPARDRGPESRPTELSLEELSISARQQPTPLTPAQPAPAPAPATTTGTRAGGHPEEPLSRLSRKRKLLEDTESGKTLLLDAYRVWQQGQKGVAYDLGRVERIMSETYMLIKQVDEEAALEQAVKFCQVHLGAAAQRQASGDTPTTPKHPKDSRENFFPVTVVPTAPDPVPADSVQRPSDAHTKPRPALAAATTIITCPPSASASTLDQSKDPGPPRPHRPEATPSMASLGPEGEELARVAEGTSFPPQEPRHSPQVKMAPTSSPAEPHCWPAEAALGTGAEPTCSQEGKLRPEPRRDGEAQEAASETQPLSSPPTAASSKAPSSGSAQPPEGHPGKPEPSRAKSRPLPNMPKLVIPSAATKFPPEITVTPPTPTLLSPKGSISEETKQKLKSAILSAQSAANVRKESLCQPALEVLETSSQESSLESETDEDDDYMDI

SEQ ID NO: 15 - DNA, 17 bp, Homo sapiens
aaagctatttataagca

SEQ ID NO: 16 - DNA, 17 bp, Homo sapiens
ttcgataaatattcgtt

SEQ ID NO: 17 - protein, 26 aa, Homo sapiens
SPKGSISEETKQKLKSAILSAQSAAN

SEQ ID NO: 18 - protein, 19 aa, Homo sapiens
KESAVATVMQEFVNKKKLA

SEQ ID NO: 19 - protein, 20 aa, Homo sapiens
KESAIATVLRQEFLKSKEPT

SEQ ID NO: 20 - protein, 18 aa, Homo sapiens
RERAVATVQEFLKSATKD

SEQ ID NO: 21 - protein, 21 aa, Homo sapiens
KCPVPFCLNIRQQQQHRLQQA

SEQ ID NO: 22 - protein, 18 aa, Artificial
  motif
XXKXXLXXKXXLXXXXXX

SEQ ID NO: 23 - protein, 36 aa, Artificial
  synthetic
RAVASTEVKQKLQEFLLSKGSGSYGRKKRRQRRRGC

SEQ ID NO: 24 - protein, 36 aa, Artificial
  synthetic
RAVASTEDKQKAQEADDSKGSGSYGRKKRRQRRRGC